(19) United States
(12) Patent Application Publication
(10) Pub. No.: US 2015/0038351 A1
(43) Pub. Date: Feb. 5, 2015

(54) Gene Signatures That Predispose Or Protect Individuals From Low-Dose Radiation Induced Breast Cancer Or Are Associated with Disease-Free Survival

(71) Applicants: Andrew J. Wyrobek, Walnut Creek, CA (US); Antoine M. Snijders, Antioch, CA (US)

(72) Inventors: Andrew J. Wyrobek, Walnut Creek, CA (US); Antoine M. Snijders, Antioch, CA (US)

(73) Assignee: THE REGENTS OF THE UNIVERSITY OF CALIFORNIA, Oakland, CA (US)

(21) Appl. No.: 14/023,968

(22) Filed: Sep. 11, 2013

Related U.S. Application Data
(60) Provisional application No. 61/801,372, filed on Mar. 15, 2013.
(60) Provisional application No. 61/699,418, filed on Sep. 11, 2012.

(51) Int. Cl. C12Q 1/68

(52) U.S. Cl. CPC C12Q 1/6886; C12Q 2600/158; C12Q 2600/16; C12Q 2600/106; C12Q 2600/118
USPC 506/9; 506/16

(57) ABSTRACT

A method for identifying sensitivity to low-dose ionizing radiation and cancer patient prognosis. Several predictor panels of genes that are predictive for increased or decreased susceptibility for LD-induced cancer and predictor panel that are predictive for increased or decreased disease free survival in women who are newly diagnosed with breast cancer.

### CROSS-REFERENCE TO RELATED APPLICATIONS

This application is a non-provisional application of and claims priority to U.S. Provisional Patent Application No. 61/801,372, filed on Mar. 15, 2013 and to U.S. Provisional Patent Application No. 61/699,418, filed on Sep. 11, 2012, both of which are hereby incorporated by reference in their entirety.

### STATEMENT OF GOVERNMENTAL SUPPORT

The invention was made with government support under Contract No. DE-AC02-05CH11231 awarded by the U.S. Department of Energy and Lawrence Berkeley National Lab Directed Research and Development (LDRD) Program funding. The government has certain rights in the invention.

### BACKGROUND OF THE INVENTION

1. Field of the Invention

The invention relates to the field of diagnostic and prognostic methods of human cancers, especially breast cancer, arising from low-dose radiation exposure and the risk of survival in women once they have been diagnosed with breast cancer.

2. Related Art

Once a woman is diagnosed with breast cancer, there is great uncertainty and variation in the chances of survival, which can be measured as disease free survival (DFS). Advanced genomic technologies provide a means to use the expression profile of tumor and nearby normal tissue to develop signatures of risks and protection for DFS and other measures of survival.

For example, human population exposures to low-dose ionizing radiation (LD, <10 cGy) are a growing medical and public health concern due to the increasing use in medical diagnostics, therapies, security screening, and exposure to emissions from nuclear power generation and unexpected events. The human breast is sensitive to radiation-induced cancer after higher doses (Mole R H (1978) The sensitivity of the human breast to cancer induction by ionizing radiation. Br J Radiol 51: 401-405) with risks depending on exposure regimen, age at exposure, and genetic background (Boice J D, Jr., Harvey E B, Blettner M, Stovall M, Flannery J T (1992) Cancer in the contralateral breast after radiotherapy for breast cancer. N Engl J Med 326: 781-785; Hancock S L, Tucker M A, Hoppe R T (1993) Breast cancer after treatment of Hodgkin's disease. J Natl Cancer Inst 85: 25-31). However, we know remarkably little of the molecular tissue responses after LD exposures, of response mechanisms that may be protective or risky for cancer, and how individuals may vary in their tissue repair and cancer risks. The consequences of these gaps in knowledge are not trivial and there can be serious public misconceptions and fears as dramatically illustrated in Japan and the rest of the world after the radiation releases from the Fukushima reactor complex after the Great East Japan Earthquake and tsunami of 2011.

Advanced genomic technologies have demonstrated the rich molecular responses in cells and tissues exposed to LD radiation in transcriptome, metabolome, epigenome, proteome and other omics. We have learned that LD responses can vary dramatically with genetic backgrounds and that there is very little overlap between LD and HD responses at the level of genes, pathways, networks, and functions (Coleman M A, Yin E, Peterson L E, Nelson D, Sorensen K, et al. (2005) Low-dose irradiation alters the transcript profiles of human lymphoblastoid cells including genes associated with cytogenetic radioadaptive response. Radiat Res 164: 369-382; Lowe X R, Bhattacharya S, Marchetti F, Wyrobek A J (2009) Early brain response to low-dose radiation exposure involves molecular networks and pathways associated with cognitive functions, advanced aging and Alzheimer's disease. Radiat Res 171: 53-65; Nguyen D H, Oketch-Rabah H A, Illa-Bochaca I, Geyer F C, Reis-Filho J S, et al. (2011) Radiation Acts on the Microenvironment to Affect Breast Carcinogenesis by Distinct Mechanisms that Decrease Cancer Latency and Affect Tumor Type. Cancer Cell 19: 640-651; Wyrobek A J, Manohar C F, Krishnan V V, Nelson D O, Furtado M R, et al. (2011) Low dose radiation response curves, networks and pathways in human lymphoblastoid cells exposed from 1 to 10 cGy of acute gamma radiation. Mutat Res 722: 119-130). Although the linear-no-threshold (LNT) model remains the regulatory standard for estimating LD risks (Health Risks From Exposure to Low Levels of Ionizing Radiation BEIR VII Phase 2. (2006) Washington, D.C.: The National Academies Press), it is under increasing scientific challenge because of the mounting evidence that many, and maybe most, cellular and tissue responses are not linear into the LD range (Wyrobek A J, Manohar C F, Krishnan V V, Nelson D O, Furtado M R, et al. (2011) Low dose radiation response curves, networks and pathways in human lymphoblastoid cells exposed from 1 to 10 cGy of acute gamma radiation. Mutat Res 722: 119-130; Neumaier T, Swenson J, Pham C, Polyzos A, Lo A T, et al. Evidence for formation of DNA repair centers and dose-response nonlinearity in human cells. Proc Natl Acad Sci USA 109: 443-448). Dose rate is also an important variable for risk, with fractionated LD and adaptive response regimens providing protection against radiation-induced cell damage, genomic damage, and cancer endpoints (Ina Y, Tanooka H, Yamada T, Sakai K (2005) Suppression of thymic lymphoma induction by life-long low-dose-rate irradiation accompanied by immune activation in C57BL/6 mice. Radiat Res 163: 153-158; Olivieri G, Bodycote J, Wolff S (1984) Adaptive response of human lymphocytes to low concentrations of radioactive thymidine. Science 223: 594-597; Ullrich R L, Jernigan M C, Satterfield L C, Bowles N D (1987) Radiation carcinogenesis: time-dose relationships. Radiat Res 111: 179-184; Wolff S (1998) The adaptive response in radiobiology: evolving insights and implications. Environ Health Perspect 106 Suppl 1: 277-283). In the mammary glands (MG) of mice, lifetime tumor incidence was associated with how the exposure was fractionated, ranging from full protection to additivity of risk (Ullrich R L et al., (1987) Radiat Res 111: 179-1). In the mouse p53-null chimera model, 10 cGy LD exposure to the MG stroma reduced tumor latency, suggesting that LD altered the tissue microenvironment (Nguyen D H, Oketch-Rabah H A, Illa-Bochaca I, Geyer F C, Reis-Filho J S, et al. (2011) Radiation Acts on the Microenvironment to Affect Breast Carcinogenesis by Distinct Mechanisms that Decrease Cancer Latency and Affect Tumor Type. Cancer Cell 19: 640-651), although 50 cGy did not show this effect, warranting further inquiry. While LD expression studies (see references above) have provided evidence for conserved as well as cell-type specific low-dose responses, the roles of genetic background on resulting tissue damage and down-stream cancer risks remain poorly understood.

### SUMMARY OF THE INVENTION

The present disclosure provides for panels of genetic probes for determining higher predicted probability of disease free survival in a patient (i.e., protective genes) and for determining lower predicted probability of disease free survival in a patient (i.e., risky genes).

In one aspect, the invention provides for panels of genetic probes that may be used for determining susceptibility to low dose (LD) ionizing radiation-induced cancer in a patient. In one embodiment, the panel comprising genetic probes to detect the genes or gene products of BMP2K, CBX7, CLASP2, PRDX2, PRDX3, RAB6B, RPS6, RUNX1 and SLC15A2 (nine biomarker panel). In another embodiment, the panel comprising genetic probes to detect the genes or gene products of MAGOHB, PAPOLA, PNPT1, POP4, PPIH, RBM39, SUPT16H, TXNL4A, EIF2S1, GNA13, GNB1, HLA-DRA, RAD23A, SMC6, ABCB10, ABCF1, BAT5, C17orf95, C19orf56, C5orf22, CAP1, CHCHD3, CHCHD4, CLDND1, DDX19A, DNAJC10, GADD45GIP1, GBP1, HLA-B, KIF5B, MCART1, MCM6, MTFR1, NRD1, PDK1, PDXDC1, PEBP1, PHF20, PI4K2B, PIGO, PPME1, SAPS3, SCAND1, SHC1, SNX6 and TM2D2 (46 biomarker panel).

In another embodiment, the panel comprising genetic probes to detect the genes or gene products of MAGOHB, PAPOLA, PNPT1, POP4, PPIH, RBM39, SUPT16H, TXNL4A, EIF2S1, GNA13, GNB1, HLA-DRA, RAD23A, SMC6, ABCB10, ABCF1, BAT5, C17orf95, C19orf56, C5orf22, CAP1, CHCHD3, CHCHD4, CLDND1, DDX19A, DNAJC10, GADD45GIP1, GBP1, HLA-B, KIF5B, MCART1, MCM6, MTFR1, NRD1, PDK1, PDXDC1, PEBP1, PHF20, PI4K2B, PIGO, PPME1, SAPS3, SCAND1, SHC1, SNX6 and TM2D2 (55 biomarker panel).

In another embodiment, the panel comprising genetic probes to detect the genes or gene products of CAD, CCNK, CDC7, CDT1, CENPH, CHEK1, EZH2, GINS1, HELLS, MCM2, MCM3, MCM4, MCM5, MCM6, MCM7, MYC, POLD1, PRIM1, RFC5, RRM2, SNRPD3, TK1, TYMS, UHRF1, WDHD1, CCDC86, ELOVL6, GABRP, KRT17, MMP12, NUP107, NUTF2, PA2G4, SLC7A5, SQLE, and WASF1 (36 biomarker panel).

Thus, a method for identifying a cancer patient with higher or lower predicted probability of disease free survival, comprising: (a) measuring the amplification or expression level of each gene in the panel in a patient sample; and (b) determining a total amplification or expression level of the panel by adding together the measurements from Step (a); and (c) comparing said total in Step (b) to a median of total amplification or expression level of said panel of genes in a normal tissue sample or a reference amplification or expression level, whereby a below-median expression level indicates a patient that has a higher predicted probability of disease free survival.

In one aspect, a method for identifying a cancer patient with higher predicted probability of disease free survival, comprising: (a) measuring the amplification or expression level of each gene in the panel in a patient sample; and (b) determining a total amplification or expression level of said panel by adding together the measurements from Step (a); and (c) comparing said total in Step (b) to a median of total amplification or expression level of the panel of genes in a normal tissue sample or a reference amplification or expression level, whereby an below-median expression of the group of genes selected from BMP2K, CBX7, CLASP2, PRDX2, PRDX3, RAB6B, RPS6, RUNX1 and SLC15A2 and an above-median expression of the group of genes selected from MAGOHB, PAPOLA, PNPT1, POP4, PPIH, RBM39, SUPT16H, TXNL4A, EIF2S1, GNA13, GNB1, HLA-DRA, RAD23A, SMC6, ABCB10, ABCF1, BAT5, C17orf95, C19orf56, C5orf22, CAP1, CHCHD3, CHCHD4, CLDND1, DDX19A, DNAJC10, GADD45GIP1, GBP1, HLA-B, KIF5B, MCART1, MCM6, MTFR1, NRD1, PDK1, PDXDC1, PEBP1, PHF20, PI4K2B, PIGO, PPME1, SAPS3, SCAND1, SHC1, SNX6 and TM2D2 indicates a patient that has a higher predicted probability of disease free survival.

In another aspect, the method further comprising a step (d) prescribing methods to reduce exposure to low dose radiation to patients with an above-median expression level. The methods to reduce exposure to low dose radiation including but not limited to such lifestyle and behavior modifications as obtaining and using extra shielding for the patient in any medical or other procedure which requires the use of radiation, or seeking an alternative to or avoiding such high energy sources used for example in airport or venue security scanners, etc.

The present disclosure also provides for a panel of genetic probes for determining susceptibility to low dose ionizing radiation (LD)-induced cancer in a patient, said panel comprising genetic probes to detect the genes or gene products of BMP2K, CBX7, CLASP2, PRDX2, PRDX3, RAB6B, RPS6, RUNX1 and SLC15A2.

In some embodiments, a method for identifying a patient with susceptibility to low dose (LD) ionizing radiation-induced cancer, comprising: (a) measuring the amplification or expression level of each gene in the biomarker panel in a sample from a patient before exposure to low dose radiation; and (b) determining a total amplification or expression level of said panel by adding together the measurements from Step (a); and (c) comparing said total in Step (b) to a median of total amplification or expression level of said panel of genes in a normal tissue sample or a reference amplification or expression level, whereby a below-median expression genes selected from the group of genes that consists of BMP2K, CBX7, CLASP2, PRDX2, PRDX3, RAB6B, RPS6, RUNX1 and SLC15A2 and an above-median expression of genes selected from the remaining genes in the panel indicates that the patient is resistant to LD-induced cancers.

In another embodiment, the present disclosure provides a 90-marker panel, consisting of all the genes in the above 4 panels minus duplicates, and used together to provide a method for detection of specific biomarkers that can identify women at increased risk for breast cancer upon exposure to low dose ionizing radiation.

In one embodiment, a panel of genetic probes for determining susceptibility to low dose ionizing radiation (LD)-induced cancer in a patient, said panel comprising genetic probes to detect the genes or gene products of MAGOHB, PAPOLA, PNPT1, POP4, PPIH, RBM39, SUPT16H, TXNL4A, EIF2S1, GNA13, GNB1, HLA-DRA, RAD23A, SMC6, ABCB10, ABCF1, BAT5, C17orf95, C19orf56, C5orf22, CAP1, CHCHD3, CHCHD4, CLDND1, DDX19A, DNAJC10, GADD45GIP1, GBP1, HLA-B, KIF5B, MCART1, MCM6, MTFR1, NRD1, PDK1, PDXDC1, PEBP1, PHF20, PI4K2B, PIGO, PPME1, SAPS3, SCAND1, SHC1, SNX6 and TM2D2. This panel thus providing for a method for identifying a patient with susceptibility to low dose ionizing radiation (LD)-induced cancer, comprising: (a) measuring the amplification or expression level of each gene probed by the panel of genetic probes in a sample from a patient before exposure to LD; and (b) determining a total amplification or expression level of said genes probed by the panel by adding together the measurements from Step (a); and (c) comparing said total in Step (b) to a median of total amplification or expression level of said genes in a normal tissue sample or a reference amplification or expression level, whereby an above-median expression indicates a patient is resistant to LD-induced cancers.

In another embodiment, a panel of genetic probes for determining higher predicted probability of disease free survival in a patient, said panel comprising genetic probes to detect the genes or gene products of MAGOHB, PAPOLA, PNPT1, POP4, PPIH, RBM39, RPS6, SUPT16H, TXNL4A, EIF2S1, GNA13, GNB1, HLA-DRA, PRDX2, PRDX3, RAD23A, RUNX1, SMC6, ABCB10, ABCF1, BAT5, BMP2K, C17orf95, C19orf56, C5orf22, CAP1, CBX7, CHCHD3, CHCHD4, CLASP2, CLDND1, DDX19A, DNAJC10, GADD45GIP1, GBP1, HLA-B, KIF5B, MCART1, MCM6, MTFR1, NRD1, PDK1, PDXDC1, PEBP1, PHF20, PI4K2B, PIGO, PPME1, RAB6B, SAPS3, SCAND1, SHC1, SLC15A2, SNX6 and TM2D2. This panel thus providing for a method for identifying a cancer patient with higher predicted probability of disease free survival, comprising: (a) measuring the amplification or expression level of each gene probed by the panel of genetic probes in a sample from a patient; and (b) determining a total amplification or expression level of said panel by adding together the measurements from Step (a); and (c) comparing said total in Step (b) to a median of total amplification or expression level of said panel of genes in a normal tissue sample or a reference amplification or expression level, whereby an below-median expression of the group of genes that consists of BMP2K, CBX7, CLASP2, PRDX2, PRDX3, RAB6B, RPS6, RUNX1 and SLC15A2 and an above-median expression of the group of genes that consists of MAGOHB, PAPOLA, PNPT1, POP4, PPIH, RBM39, SUPT16H, TXNL4A, EIF2S1, GNA13, GNB1, HLA-DRA, RAD23A, SMC6, ABCB10, ABCF1, BAT5, C17orf95, C19orf56, C5orf22, CAP1, CHCHD3, CHCHD4, CLDND1, DDX19A, DNAJC10, GADD45GIP1, GBP1, HLA-B, KIF5B, MCART1, MCM6, MTFR1, NRD1, PDK1, PDXDC1, PEBP1, PHF20, PI4K2B, PIGO, PPME1, SAPS3, SCAND1, SHC1, SNX6 and TM2D2 indicates a patient that has a higher predicted probability of disease free survival.

In another embodiment, a panel of genetic probes for determining susceptibility to low dose ionizing radiation (LD)-induced cancer in a patient, said panel comprising genetic probes to detect the genes or gene products of MAGOHB, PAPOLA, PNPT1, POP4, PPIH, RBM39, RPS6, SUPT16H, TXNL4A, EIF2S1, GNA13, GNB1, HLA-DRA, PRDX2, PRDX3, RAD23A, RUNX1, SMC6, ABCB10, ABCF1, BAT5, BMP2K, C17orf95, C19orf56, C5orf22, CAP1, CBX7, CHCHD3, CHCHD4, CLASP2, CLDND1, DDX19A, DNAJC10, GADD45GIP1, GBP1, HLA-B, KIF5B, MCART1, MCM6, MTFR1, NRD1, PDK1, PDXDC1, PEBP1, PHF20, PI4K2B, PIGO, PPME1, RAB6B, SAPS3, SCAND1, SHC1, SLC15A2, SNX6 and TM2D2. This panel of genetic probes thus providing for a method for identifying a patient with susceptibility to low dose ionizing radiation (LD)-induced cancer, comprising: (a) measuring the amplification or expression level of each gene probed by the panel of genetic probes in a sample from a patient before exposure to LD; and (b) determining a total amplification or expression level of said panel by adding together the measurements from Step (a); and (c) comparing said total in Step (b) to a median of total amplification or expression level of the genes of probed by the panel of genetic probes in a normal tissue sample or a reference amplification or expression level, whereby an below-median expression of the group of genes that consists of BMP2K, CBX7, CLASP2, PRDX2, PRDX3, RAB6B, RPS6, RUNX1 and SLC15A2 and an above-median expression of the group of genes that consists of MAGOHB, PAPOLA, PNPT1, POP4, PPIH, RBM39, SUPT16H, TXNL4A, EIF2S1, GNA13, GNB1, HLA-DRA, RAD23A, SMC6, ABCB10, ABCF1, BAT5, C17orf95, C19orf56, C5orf22, CAP1, CHCHD3, CHCHD4, CLDND1, DDX19A, DNAJC10, GADD45GIP1, GBP1, HLA-B, KIF5B, MCART1, MCM6, MTFR1, NRD1, PDK1, PDXDC1, PEBP1, PHF20, PI4K2B, PIGO, PPME1, SAPS3, SCAND1, SHC1, SNX6 and TM2D2 indicates a patient is resistant to LD-induced cancers.

In one embodiment, a panel of genetic probes for determining higher predicted probability of disease free survival in a patient, said panel comprising genetic probes to detect the genes or gene products of CAD, CCNK, CDC7, CDT1, CENPH, CHEK1, EZH2, GINS1, HELLS, MCM2, MCM3, MCM4, MCM5, MCM6, MCM7, MYC, POLD1, PRIM1, RFC5, RRM2, SNRPD3, TK1, TYMS, UHRF1, WDHD1, CCDC86, ELOVL6, GABRP, KRT17, MMP12, NUP107, NUTF2, PA2G4, SLC7A5, SQLE, and WASF1. This panel of genetic probes thus providing for a method for identifying a cancer patient with higher predicted probability of disease free survival, comprising: (a) measuring the amplification or expression level of each gene in said panel of Claim 16 in a sample from a patient; and (b) determining a total amplification or expression level of said panel by adding together the measurements from Step (a); and (c) comparing said total in Step (b) to a median of total amplification or expression level of said panel of genes in a normal tissue sample or a reference amplification or expression level, whereby an above-median expression post-LD exposure indicates a patient that has a lower predicted probability of disease free survival and poor prognosis.

In yet another embodiment, a panel of genetic probes for determining susceptibility to low dose ionizing radiation (LD)-induced cancer in a patient, said panel comprising genetic probes to detect the genes or gene products of CAD, CCNK, CDC7, CDT1, CENPH, CHEK1, EZH2, GINS1, HELLS, MCM2, MCM3, MCM4, MCM5, MCM6, MCM7, MYC, POLD1, PRIM1, RFC5, RRM2, SNRPD3, TK1, TYMS, UHRF1, WDHD1, CCDC86, ELOVL6, GABRP, KRT17, MMP12, NUP107, NUTF2, PA2G4, SLC7A5, SQLE, and WASF1. This panel of genetic probes thus providing for a method for identifying a patient with susceptibility to low dose ionizing radiation (LD)-induced cancer, comprising: (a) measuring the amplification or expression level of each gene in said panel of claim 18 in a sample from a patient before exposure to LD; and (b) determining a total amplification or expression level of said panel by adding together the measurements from Step (a); and (c) comparing said total in Step (b) to a median of total amplification or expression level of said panel of genes in a normal tissue sample or a reference amplification or expression level, whereby an above-median expression post-LD exposure indicates a patient that is sensitive to LD-induced cancers.

In another embodiment, a panel of genetic probes for determining higher predicted probability of disease free survival in a patient, said panel comprising genetic probes to detect the genes or gene products of ABCB10, ABCF1, BAT5, BMP2K, C17orf95, C19orf56, C5orf22, CAD, CAP1, CBX7, CCDC86, CCNK, CDC7, CDT1, CENPH, CHCHD3, CHCHD4, CHEK1, CLASP2, CLDND1, DDX19A, DNAJC10, EIF2S1, ELOVL6, EZH2, GABRP, GADD45GIP1, GBP1, GINS1, GNA13, GNB1, HELLS, HLA-B, HLA-DRA, KIF5B, KRT17, MAGOHB, MCART1, MCM2, MCM3, MCM4, MCM5, MCM6, MCM7, MMP12, MTFR1, MYC, NRD1, NUP107, NUTF2, PA2G4, PAPOLA, PDK1, PDXDC1, PEBP1, PHF20, PI4K2B, PIGO, PNPT1, POLD1, POP4, PPIH, PPME1, PRDX2, PRDX3, PRIM1, RAB6B, RAD23A, RBM39, RFC5, RPS6, RRM2, RUNX1, SAPS3, SCAND1, SHC1, SLC15A2, SLC7A5, SMC6, SNRPD3, SNX6, SQLE, SUPT16H, TK1, TM2D2, TXNL4A, TYMS, UHRF1, WASF1 and WDHD1.

In another embodiment, a panel of genetic probes for determining susceptibility to low dose ionizing radiation (LD)-induced cancer in a patient, said panel comprising genetic probes to detect the genes or gene products of ABCB10, ABCF1, BAT5, BMP2K, C17orf95, C19orf56, C5orf22, CAD, CAP1, CBX7, CCDC86, CCNK, CDC7, CDT1, CENPH, CHCHD3, CHCHD4, CHEK1, CLASP2, CLDND1, DDX19A, DNAJC10, EIF2S1, ELOVL6, EZH2, GABRP, GADD45GIP1, GBP1, GINS1, GNA13, GNB1, HELLS, HLA-B, HLA-DRA, KIF5B, KRT17, MAGOHB, MCART1, MCM2, MCM3, MCM4, MCM5, MCM6, MCM7, MMP12, MTFR1, MYC, NRD1, NUP107, NUTF2, PA2G4, PAPOLA, PDK1, PDXDC1, PEBP1, PHF20, PI4K2B, PIGO, PNPT1, POLD1, POP4, PPIH, PPME1, PRDX2, PRDX3, PRIM1, RAB6B, RAD23A, RBM39, RFC5, RPS6, RRM2, RUNX1, SAPS3, SCAND1, SHC1, SLC15A2, SLC7A5, SMC6, SNRPD3, SNX6, SQLE, SUPT16H, TK1, TM2D2, TXNL4A, TYMS, UHRF1, WASF1 and WDHD1.

In another embodiment, the invention also provides for four proliferation-independent expression signatures and tumor-stromal interaction mechanisms associated with disease-free survival in breast cancer patients.

In some embodiments, a panel comprising genetic probes to detect the genes or gene products of one to four categories of genes. In one embodiment, a panel of genetic probes for determining Category 1 risky genes associated with cellular proliferation status in a patient, said panel comprising genetic probes to detect the genes or gene products of RACGAP1, RFK, MTCH1, SCAND1, PRC1, HRASLS, ZWINT, EPRS. In another embodiment, a panel of genetic probes for determining Category 2 risky genes not associated with cellular proliferation status in a patient, said panel comprising genetic probes to detect the genes or gene products of ABCC5, COX7A2, KIAA0895, P4HA2, GPR56, RECQL4, PCDH17, DHX15, HSBP1, PPP3CA, DHFR.

In another embodiment, a panel of genetic probes for determining Category 3 protective genes associated with cellular proliferation status in a patient, said panel comprising genetic probes to detect the genes or gene products of SIK3, PTGDS, FAM21B, KIAA0040, ARID5B, ID1, PELI2, CX3CR1, SGK3. In another embodiment, a panel of genetic probes for determining Category 4 protective genes not associated with cellular proliferation in a patient, said panel comprising genetic probes to detect the genes or gene products of CD1C, SPTAN1, AHCYL1, GLB1L, CD302, EPHA1, IGLL1/IGLL5.

A method for identifying a cancer patient with higher predicted probability of disease free survival, comprising: (a) measuring the amplification or expression level of each gene in a Category 1 or Category 2 risky gene panel in a sample from a patient; and (b) determining individual and total amplification or expression level of said genes in the panel by adding together the measurements from Step (a); and (c) comparing said total in Step (b) to a median of total amplification or expression level of said panel of genes in a normal tissue sample or a reference amplification or expression level, whereby a below-median expression level indicates a patient that has a higher predicted probability of disease free survival and an above-median expression level indicates a patient that has a lower predicted probability of disease free survival.

A method for identifying a cancer patient with higher predicted probability of disease free survival, comprising: (a) measuring the amplification or expression level of each gene in a Category 3 or Category 4 protective gene panel in a sample from a patient; and (b) determining individual and total amplification or expression level of said genes in the panel by adding together the measurements from Step (a); and (c) comparing said total in Step (b) to a median of total amplification or expression level of said panel of genes in a normal tissue sample or a reference amplification or expression level, whereby an above-median expression level indicates a patient that has a higher predicted probability of disease free survival, and a below-median expression level indicates a patient that has a lower predicted probability of disease free survival.

### BRIEF DESCRIPTION OF THE FIGURES

FIG. 1. Radiation induced micronuclei in erythrocytes of mice that differ in LD-induced mammary cancer sensitivity. A. BALB/c mice are sensitive to radiation induced mammary gland, lung and ovarian cancer, whereas C57BL/6 mice are more resistant [14]. Mice were divided into three exposure groups: (1) low dose group: four weekly doses of 7.5 cGy, (2) high dose reference group: four weekly doses of 1.8 Gy and (3) unexposed group: four weekly sham irradiations. All mice were approximately 8 wks of age at the start of the radiation regimen. Saphenous vein blood was collected for micronucleus analysis at 6 days after the third dose (−1 day in relation to the 4^{th} dose), 6 days after the 4^{th} dose, and 1 month after the 4^{th} dose. Mammary gland tissues were collected for microarray and molecular analyses at 4 hrs and 1 month after the last exposure. B. Relative frequencies of MN-RETs in high (left) and low (right) dose groups of C57BL/6 (black bars) and BALB/c mice (white bars). Bars outlined in red indicate sham-irradiated controls. C. Relative frequencies of MN-NCEs in high (left) and low (right) dose groups of C57BL/6 (black bars) and BALB/c mice (white bars). Significance was tested by ANOVA with Dunnett adjustment for multiple comparisons. An asterisk indicates significant difference in treated groups compared to the respective frequencies in sham of that same strain (* p<0.0001; ** p<0.02). The number sign (#) indicates significant baseline differences in MN RET and NCE among strains (p<0.0001).

FIG. 2. Genetic differences in baseline gene expression are correlated among tissues and associated with diverse functions. A. Transcript profiles of BALB/c and C57BL/6 mice in mammary glands and blood identified 131 genes with the same relative ratio of expression in mammary gland and blood (r²=0.83). B. Distributions of functions (GO—biological processes) associated with the 131-gene set.

FIG. 3. Radiation responses are dependent on genotype, dose and time after exposure. Early and 1-month response gene lists were generated based on (fold-change=1.5 fold; p-value ≦0.01 for high dose; ≦0.1 for low dose). BALB/c mice in white, C57BL/6 in black, overlap in orange.

FIG. 4. Unique early LD radiation response pathways and networks in mammary glands of BALB/c and C57BL/6. A. Early (4 hr) response for canonical pathways in BALB/c and C57BL/6 mice after low (L) or high (H) dose radiation. Downregulated pathways in green; upregulated pathways in red; pathways containing both have diagonal line; pathways not reaching statistical significance in gray. B. HIF1A Subnetwork. Protein network centered on HIF1A in the BALB/c. Downregulated genes in green; upregulated genes in red. C. Immunohistochemical (IHC) analyses of mammary glands of sham and low-dose irradiated BALB/c mice. In sham irradiated BALB/c mice at least 35% of mammary gland ducts have a surrounding macrophage. In contrast, in low-dose irradiated BALB/c mice, no macrophages were observed in 3/3 animals. A representative image of IHC analysis of EMR1 (F4/80) protein in the mammary gland of a sham irradiated BALB/c mouse is shown on the right.

FIG. 5. Unique 1-month LD radiation response pathways and networks in mammary glands of BALB/c and C57BL/6. A. 1-Month LD responses for canonical pathways in BALB/c and C57BL/6 mice (See legend FIG. 4A). B. 1-Month LD response mitotic gene enriched interaction networks. Mitosis gene enriched interaction networks in MG of BALB/c (left) and C57BL/6 (right) mice. In the BALB/c network the majority of genes were upregulated (red) and in the C57BL/6 network the majority of genes were downregulated (green).

FIG. 6. Reduction in SOX9-labelled luminal and myoepithelial cells after LD exposure in C57BL/6 mammary glands. A. The percent SOX9 positive luminal and myoepithelial cells are significantly reduced in low-dose irradiated mammary glands 1-month after low-dose exposure (p<0.0001). B. Representative images of immunohistochemical analysis of SOX9 protein in mammary glands of sham (left) or LD (right) irradiated C57BL/6 mice. Myoepithelial cells were defined as those cells directly surrounding the luminal cell layer. Counterstaining was performed using hematoxylin. Note positive staining is limited to the nucleus in cells of luminal or myoepithelial origin.

FIG. 7. Unbiased baseline and 1-month BALB/c LD signatures are associated with human breast cancer disease-free survival. The full set of significantly differentially expressed genes between BALB/c and C57BL/6 in blood and mammary gland (left) and one-month LD up-regulated BALB/c genes (right) were used to calculate the overall sum of expression values of the same genes in human breast tumors (n=159). A. For both signatures patients with sum expression above the group median expression had a worse prognosis than patients below the median. B. Kaplan-Meier disease-free survival curves indicate that patients with above median survival have a worse 10-year survival compared to patients with below median survival in two independent data-sets (top: GSE1456, bottom: GSE1456 and GSE6532).

FIG. 8. Concordance of expression between 1-month mammary gland LD radiation responses and human breast cancer signatures. A. Comparison of directionality of expression of 1-month low dose genes in BALB/c and C57BL/6 mice that overlap with 946 human breast cancer biomarkers with expression in DCIS and breast cancer. Upregulated genes in red; downregulated genes in green. Genes with a diagonal line had evidence for both up- and downregulation. B. A human poor prognosis signature compared against expression of 1-month low dose genes in BALB/c and C57BL/6 response genes.

FIG. 9. Integrative model of genetic differences in tissue functions in radiation-sensitive and resistant mice. Unirradiated BALB/c mice have significantly higher levels of micronucleated cells compared to C57BL/6. Strain differences in gene expression are associated with RNA processing and stress response and associated expression signatures are associated with poor survival in breast cancer patients. The early (4-hr) LD response in BALB/c mice is largely driven by TGFbeta activation, HIF1A, and immune deficiency. These functions are not detected in the early expression profiles of C57BL/6 mice. At 1-month after LD irradiation, BALB/c MGs exhibits increased expression of transcriptional regulators associated with proliferation, senescence-like, and cancer-associated functions, while C57BL/6 exhibits decreased expression of proliferation-associated genes.

FIG. 10. Tissue functions associated with the early and 1-month LD response in BALB/c and C57BL/6 mammary glands.

Distributions of predicted functions (based on GO, KEGG, IPA Canonical Pathways, Genes of Interest) at early (4 hr) and 1-month sampling times in both strains. Numbers of genes used to generate charts are listed (unique genes in parentheses).

FIG. 11. Early LD radiation response networks in the mammary glands of BALB/c mice show broad downregulation of immune response genes.

The top two protein interaction networks for the BALB/c low dose genes include the functions of Inflammatory Disease/Cell Mediated Immune Response/Cellular Movement (top) and Cellular Growth, Proliferation/Hematological System Development, and Function/Humoral Immune Response (bottom). Upregulated and downregulated genes are represented in red and green, respectively.

FIG. 12. Genetic differences in protein-interaction networks in mammary glands of BALB/c and C57BL/6 mice at 1-month after LD exposures. Gene interaction network enriched for genes involved in DNA replication containing mostly MCM family genes were found to contain mostly upregulated genes (highlighted in red) in mammary gland of BALB/c mice at 1 month after low dose radiation exposure. B. Protein interaction analyses of the mammary gland of C57BL/6 mice at 1-month after low dose radiation show an extracellular-matrix network and a keratin-enriched network. Genes upregulated after low dose radiation are shown in red and downregulated genes in green. In each of these networks, the majority of genes were downregulated.

FIG. 13. Early expression non-linearities in the mammary glands of BALB/c mice exposed to LD radiation. Fold changes in responses were calculated with respect to sham-irradiated mammary glands for genes modulated after low dose and high dose radiation exposures, represented in blue and orange, respectively. Genes were filtered on fold change (+/−1.5) and p-value (<0.1 low dose and <0.05 high dose). 76 BALB/c genes were modulated in the same direction and at similar magnitudes after low-versus high-dose exposures (left two panels). These genes are enriched for DNA metabolism (p=0.002), DNA replication (p=0.009) and immune responses (p<0.05) as well as mitosis. Note the large cluster of 35 genes that was differentially modulated, i.e., upregulated after low dose and downregulated after high dose (far right panel). B. Mammary epithelial markers with opposite direction of response after low and high dose exposures in BALB/c.

FIG. 14. Approach and workflow for determining the 4 categories of risk: 1—Risky-aff, 2—Risky-unaff, 3—Protective-aff and 4—Protective-Unaff genes.

FIG. 15A shows two tables. The top table showing the five human breast cancer expression and outcome knowledgebases used and the bottom table shows the vital status and survival times of the patients in each of the databases. FIG. 15B shows the distribution of patients by tumor subtype.

FIG. 16A. shows overview of the four categories found by the Hazard Ratio and Cox stepwise regression analysis. FIG. 16B shows the algorithm used for the hazard Ratio and regression analysis. FIG. 16C shows the Hazard Ratio for a “Risky” gene. FIG. 16D shows the Hazard Ratio for a “Protective” gene.

FIG. 17 shows how each human gene was ranked by the number of times it was significantly risky or protective for survival among the 5 human breast cancer databases.

FIG. 18. Example of expression distribution of probe set 201705_at(PSMD7).

FIGS. 19A, 19B and 19C show the van der Waerden scores of inter-individual variation in the expression of the 4 signatures by tumor subtype. The two signatures affected by proliferation (FIGS. 19A and 19C) show differences among the tumor subtypes with apparent anti-symmetry between the Risky and Protective signatures within subtype. The distributions of the two signatures unaffected by proliferation status (FIGS. 19B and 19D) appear to be independent of tumor subtype.

FIG. 20. Overall K-M Plots for the 4 signature among 5 databases.

FIG. 21 K-M Plots for Risky-aff signature

FIG. 22. K-M Plots for Risky-Unaff signature

FIG. 23. K-M Plots for Protective-Aff signature

FIG. 24. K-M Plots for the Protective-Unaff Signature

FIG. 25. Chi-Squares of K-M analysis for 4 signatures among major tumor subtypes.

FIG. 26. K-M Plots for the 4 signature in Basal tumor subtypes.

FIG. 27. K-M Plots for the 4 signature in HER2 tumor subtypes

FIG. 28. K-M Plots for the 4 signature in Luminal tumor subtypes

FIG. 29. K-M Plots for the 4 signature in Luminal B tumor subtypes

FIG. 30. K-M Plots for the 4 signature in Normal-like tumor subtypes

FIGS. 31A, 31B, 31C, and 31D. Networks, pathways and tissue functions associated with the Category 1 Risky genes affected by proliferation status. FIG. 31A shows the network scores and p-values. FIG. 31B shows the networks for RISKY-aff genes are associated with proliferation and mitochondrial function. FIG. 31C shows the networks and graphs that demonstrate that RISKY-aff genes are associated with cell cycle processes.

FIGS. 32A, 32B, and 32C. Networks, pathways and tissue functions associated with the Category 2 Risky genes NOT affected by proliferation status. FIG. 32A shows the network scores and p-values. FIG. 32B shows the networks for RISKY-unaff genes are associated with skeletal and muscular disorders. FIG. 32C shows the networks and graphs that demonstrate that RISKY-aff genes are associated with oxidative phosphorylation and mitochondrial dysfunction.

FIGS. 33A, 33B, and 33C. Networks, pathways and tissue functions associated with the Category 3 Protective genes affected by proliferation status. FIG. 33A shows the network scores and p-values. FIG. 33B shows the networks for PROTECTIVE-aff genes are associated with connective tissue growth. FIG. 33C shows the networks and graphs that demonstrate that Protective-aff genes are associated with locomotion, chemokine signaling and lipid metabolism.

FIGS. 34A and 34B. Networks, pathways and tissue functions associated with the Category 4 Protective genes NOT affected by proliferation status. FIG. 34A shows the network scores and p-values. FIG. 34B shows the networks for PROTECTIVE-unaff genes are associated with a variety of signaling pathways.

The markers that are associated with mammary epithelium are upregulated after low dose radiation, whereas after high dose radiation the same genes were found to be downregulated.

Table 1. The early LD radiation response in BALB/c mice is mediated by TGF-β, involves inappropriate expression of mammary development genes, and involves breast cancer associated genes.

Table 2. Cancer Outcome Associated (COA) genes in the baseline and 1-month BALB/c LD signatures are defined by their significant association with breast cancer disease-free survival. Table 2a lists COA Genes* of the Systemic Baseline Signature (n=55) and Table 2b lists COA Genes* of the BALB/c 1 month LD Signature (n=36). *Cancer Outcome Associated (COA) genes were identified as follows. For each gene in the unbiased signature, a t-test was applied comparing average expression in the above median patient group vs the below median patient group. Patients were assigned to each group based on median expression of all baseline (94 genes) or BALB/c 1 mo up genes (96 genes). For the COA signature all genes with p<0.01 that were expressed at higher or lower (genes in italic) levels in the above median patient group are listed here (n=55 genes). For the 1 month BALB/c up signature all genes with p<0.01 that were expressed at higher levels in the above median patient group are listed here (n=36 genes).

Table 3. Strain differences in the induction of LD ‘thresholded’ genes as evidence for genetic differences in LD response mechanisms

Table 4. Baseline levels of micronucleated reticulocytes (RET) and normochromatic erythrocytes (NCE) are significantly higher in BALB/c compared to C57BL/6 mice.

Table 5. Time-course of micronuclei (MN) induction in blood reticulocytes (RET) and normochromatic erythrocytes (NCE) of C57BL/6 female mice after fractionated exposures to low and high doses of ionizing radiation.

Table 6. Time-course of micronucleus (MN) induction in blood reticulocytes (RET) and normochromatic erythrocytes (NCE) of BALB/c female mice after fractionated exposures to low and high doses of ionizing radiation.

Table 7. Canonical pathways significantly modulated after fractionated low or high dose exposures in the mammary gland of BALB/c or C57B1/6. Table 7a reflects Early (4 hr) Response. Table 7b reflects 1-Month Reponse.

Table 8. Quantitative RT PCR confirmations of microarray findings.

Table 9. Baseline 9-marker panel, representing a subset of Cancer Outcome Associated genes from the 55-marker baseline panel that are significantly associated with disease free survival.

Table 10. Four categories of genes and the 8-10 genes that make up the panel to identify patients having one of the four profiles.

Table 11. Four categories of genes and the 8-10 gene panels with their Unigene and Gene IDs.

### DESCRIPTION OF THE PREFERRED EMBODIMENTS

Herein is described systems, methods and devices and kits for the identification of multiple panels of genes whose gene expression levels in part provide signatures associated with the risk of a patient's disease free survival. In some embodiments, a 9-gene panel signature described is associated with a patient's susceptibility for low dose ionizing radiation-induced tissue damage and disease-free survival. In other embodiments, four signatures describe risky and protective genes that are statistically associated with the duration of disease free survival (DFS) in women diagnosed with breast cancer.

Thus, the present panels can also be used to determine higher predicted probability of disease free survival in a patient in relation to various types of cancers including but not limited to epithelial cancers such as breast, pancreatic, lung, cervical, ovarian, prostate, non-small cell lung carcinomas, melanomas, squamous cell cancers, etc.

The present methods describe the measurement and detection of expression levels of a gene as measured from a sample from a patient that comprises essentially a cancer cell or cancer tissue of a cancer tumor. Such methods for obtaining such samples are well known to those skilled in the art. For example, when the cancer is breast cancer, the expression level of a gene is measured from a sample from the patient and the sample comprising a breast cancer cell or breast cancer tissue of a breast cancer tumor.

Methods for detection of expression levels of a gene can be carried out using known methods in the art including but not limited to, fluorescent in situ hybridization (FISH), immunohistochemical analysis, fluorescence detection, comparative genomic hybridization, PCR methods including real-time and quantitative PCR, mass and imaging spectrometry and spectroscopy methods and other sequencing and analysis methods known or developed in the art. The expression level of the gene in question can be measured by measuring the amount or number of molecules of mRNA or transcript in a cell. The measuring can comprise directly measuring the mRNA or transcript obtained from a cell, or measuring the cDNA obtained from an mRNA preparation thereof. Such methods of extracting the mRNA or transcript from a cell, or preparing the cDNA thereof are well known to those skilled in the art. In other embodiments, the expression level of a gene can be measured by measuring or detecting the amount of protein or polypeptide expressed, such as measuring the amount of antibody that specifically binds to the protein in a dot blot or Western blot. The proteins described in the present invention can be overexpressed and purified or isolated to homogeneity and antibodies raised that specifically bind to each protein. Such methods are well known to those skilled in the art.

Typically, the expression levels of all genes in a panel are determined. However, in some embodiments, the expression level of fewer genes, e.g., 5, 6, 7, 8, 9, 10, 11, 12, 13, 14, 15, . . . 20, 25, 30, 36, 40, or up to 46 genes, may be evaluated. Gene expression levels may be measured using any number of methods known in the art. In typical embodiments, the method involves measuring the level of RNA. RNA expression can be quantified using any method, e.g., employing a quantitative amplification method such as qPCR. In other embodiments, the methods employ array-based assays. In still other embodiments, protein products may be detected. The gene expression patterns are determined using a sample typically obtained from a patient biopsy.

Comparison of the detected expression level of a gene in a patient sample is often compared to the expression levels detected in a normal tissue sample or a reference expression level. In some embodiments, the reference expression level can be the average or normalized expression level of the gene in a panel of normal cell lines or cancer cell lines.

In various embodiments, the expression levels of genes in the given biomarker panel are determined for identifying a cancer patient with higher predicted probability of disease free survival, comprising: (a) measuring the amplification or expression level of each gene in one of the biomarker panels in a sample from a patient; and (b) determining a total amplification or expression level of said panel by adding together the measurements from Step (a); and (c) comparing said total in Step (b) to a median of total amplification or expression level of said panel of genes in a normal tissue sample or a reference amplification or expression level, whereby a below-median expression level indicates a patient that has a higher predicted probability of disease free survival.

Gene sequences and gene products that may be detected are herein identified by gene name, Unigene ID, GeneID and/or GenBank Accession Numbers, and the publicly available content all of which are hereby incorporated by reference in their entireties for all purposes. As used herein, a “gene set forth in” a table or a panel or a “gene identified in” a table, and the like, are used interchangeably to refer to the gene that is listed in that table. For example, a gene “identified in” Table 9 refers to the gene that corresponds to the gene listed in Table 9. As understood in the art, there are naturally occurring polymorphisms for many gene sequences. Genes that are naturally occurring allelic variations for the purposes of this invention are those genes encoded by the same genetic locus. The proteins encoded by allelic variations of any gene set forth in Table 2 (or in any of Tables 2, 9 or Table 10-11) typically have at least 95% amino acid sequence identity to one another, i.e., an allelic variant of a gene indicated in Table 2 typically encodes a protein product that has at least 95% identity, often at least 96%, at least 97%, at least 98%, or at least 99%, or greater, identity to the amino acid sequence encoded by the nucleotide sequence denoted by the Entrez Gene ID number (as of Sep. 12, 2012 for Tables 2 and 9 genes and as of Mar. 15, 2013 for Tables 10-11 genes) shown in Table 2 for that gene. For example, an allelic variant of a gene encoding CBX7 (gene: chromobox homolog 7) typically has at least 95% identity, often at least 96%, at least 97%, at least 98%, or at least 99%, or greater, to the CBX7 protein sequence encoded by the nucleic acid sequence available under Entrez Gene ID no. 23492). In some cases, a “gene identified in” a table, such as Table 2, 9, 10-11, may also refer to an isolated polynucleotide that can be unambiguously mapped to the same genetic locus as that of a gene assigned to a genetic locus by the Entrez Gene ID or it may also refer to an expression product that is encoded by a polynucleotide that can be unambiguously mapped to the same genetic locus as that of a gene assigned to a genetic locus by the Entrez Gene ID.

In some embodiments, a panel of genetic probes that is described herein comprises genetic probes which detects one of the sequences of interest and optionally linked to a solid support. The quantity of RNA encoded by a gene set forth in any of the listed panels or in Tables 2a, 2b, 9, 10 or 11, can be readily determined according to any method known in the art for quantifying RNA. Various methods involving amplification reactions and/or reactions in which probes are linked to a solid support and used to quantify RNA may be used. Alternatively, the RNA may be linked to a solid support and quantified using a probe to the sequence of interest.

An “RNA nucleic acid sample” analyzed in the invention is obtained from a tumor sample obtained from the patient. An “RNA nucleic acid sample” comprises RNA, but need not be purely RNA, e.g., DNA may also be present in the sample. Techniques for obtaining an RNA sample from tumors are well known in the art.

In some embodiments, the target RNA is first reverse transcribed and the resulting cDNA is quantified. In some embodiments, RT-PCR or other quantitative amplification techniques are used to quantify the target RNA. Amplification of cDNA using PCR is well known (see U.S. Pat. Nos. 4,683,195 and 4,683,202; PCR PROTOCOLS: A GUIDE TO METHODS AND APPLICATIONS (Innis et al., eds, 1990)). Methods of quantitative amplification are disclosed in, e.g., U.S. Pat. Nos. 6,180,349; 6,033,854; and 5,972,602, as well as in, e.g., Gibson et al., Genome Research 6:995-1001 (1996); DeGraves, et al., Biotechniques 34(1):106-10, 112-5 (2003); Deiman B, et al., Mol Biotechnol. 20(2):163-79 (2002). Alternative method for determining the level of a mRNA of interest in a sample may involve other nucleic acid amplification methods such as ligase chain reaction (Barany (1991) Proc. Natl. Acad. Sci. USA 88:189-193), self-sustained sequence replication (Guatelli et al. (1990) Proc. Natl. Acad. Sci. USA 87:1874-1878), transcriptional amplification system (Kwoh et al. (1989) Proc. Natl. Acad. Sci. USA 86:1173-1177), Q-Beta Replicase (Lizardi et al. (1988) Bio/Technology 6:1197), rolling circle replication (U.S. Pat. No. 5,854,033) or any other nucleic acid amplification method, followed by the detection of the amplified molecules using techniques well known to those of skill in the art.

In general, quantitative amplification is based on the monitoring of the signal (e.g., fluorescence of a probe) representing copies of the template in cycles of an amplification (e.g., PCR) reaction. One method for detection of amplification products is the 5′-3′ exonuclease “hydrolysis” PCR assay (also referred to as the TaqMan™ assay) (U.S. Pat. Nos. 5,210,015 and 5,487,972; Holland et al., PNAS USA 88: 7276-7280 (1991); Lee et al., Nucleic Acids Res. 21: 3761-3766 (1993)). This assay detects the accumulation of a specific PCR product by hybridization and cleavage of a doubly labeled fluorogenic probe (the “TaqMan™” probe) during the amplification reaction. The fluorogenic probe consists of an oligonucleotide labeled with both a fluorescent reporter dye and a quencher dye. During PCR, this probe is cleaved by the 5′-exonuclease activity of DNA polymerase if, and only if, it hybridizes to the segment being amplified. Cleavage of the probe generates an increase in the fluorescence intensity of the reporter dye. Another method of detecting amplification products that relies on the use of energy transfer is the “beacon probe” method described by Tyagi and Kramer, Nature Biotech. 14:303-309 (1996), which is also the subject of U.S. Pat. Nos. 5,119,801 and 5,312,728. This method employs oligonucleotide hybridization probes that can form hairpin structures.

Various other techniques for performing quantitative amplification of nucleic acids are also known. For example, some methodologies employ one or more probe oligonucleotides that are structured such that a change in fluorescence is generated when the oligonucleotide(s) is hybridized to a target nucleic acid. For example, one such method involves is a dual fluorophore approach that exploits fluorescence resonance energy transfer (FRET), e.g., LightCycler™ hybridization probes, where two oligo probes anneal to the amplicon. The oligonucleotides are designed to hybridize in a head-to-tail orientation with the fluorophores separated at a distance that is compatible with efficient energy transfer. Other examples of labeled oligonucleotides that are structured to emit a signal when bound to a nucleic acid or incorporated into an extension product include: Scorpions™ probes (e.g., Whitcombe et al., Nature Biotechnology 17:804-807, 1999, and U.S. Pat. No. 6,326,145), Sunrise™ (or Amplifluor™) probes (e.g., Nazarenko et al., Nuc. Acids Res. 25:2516-2521, 1997, and U.S. Pat. No. 6,117,635), and probes that form a secondary structure that results in reduced signal without a quencher and that emits increased signal when hybridized to a target (e.g., Lux Probes™)□.

In other embodiments, intercalating agents that produce a signal when intercalated in double stranded DNA may be used. Exemplary agents include SYBR GREEN™ and SYBR GOLD™. Since these agents are not template-specific, it is assumed that the signal is generated based on template-specific amplification. This can be confirmed by monitoring signal as a function of temperature because melting point of template sequences will generally be much higher than, for example, primer-dimers, etc.

In other embodiments, the mRNA is immobilized on a solid surface and contacted with a probe, e.g., in a dot blot or Northern format. In an alternative embodiment, the probe(s) are immobilized on a solid surface and the mRNA is contacted with the probe(s), for example, in a gene chip array. A skilled artisan can readily adapt known mRNA detection methods for use in detecting the level of mRNA encoding the biomarkers or other proteins of interest.

In some embodiments, microarrays, e.g., are employed. DNA microarrays provide one method for the simultaneous measurement of the expression levels of large numbers of genes. Each array consists of a reproducible pattern of capture probes attached to a solid support. Labeled RNA or DNA is hybridized to complementary probes on the array and then detected by laser scanning. Hybridization intensities for each probe on the array are determined and converted to a quantitative value representing relative gene expression levels. See, U.S. Pat. Nos. 6,040,138, 5,800,992 and 6,020,135, 6,033,860, and 6,344,316. High-density oligonucleotide arrays are particularly useful for determining the gene expression profile for a large number of RNA's in a sample.

Techniques for the synthesis of these arrays using mechanical synthesis methods are described in, e.g., U.S. Pat. No. 5,384,261. Although a planar array surface is often employed the array may be fabricated on a surface of virtually any shape or even a multiplicity of surfaces. Arrays may be peptides or nucleic acids on beads, gels, polymeric surfaces, fibers such as fiber optics, glass or any other appropriate substrate, see U.S. Pat. Nos. 5,770,358, 5,789,162, 5,708,153, 6,040,193 and 5,800,992. Arrays may be packaged in such a manner as to allow for diagnostics or other manipulation of an all-inclusive device.

Primer and probes for use in amplifying and detecting the target sequence of interest can be selected using well-known techniques.

In some embodiments, the methods of the invention further comprise detecting level of expression of one or more reference genes that can be used as controls to determine expression levels. Such genes are typically expressed constitutively at a high level and can act as a reference for determining accurate gene expression level estimates.

In some embodiments, e.g., where the expression level of a protein encoded by a biomarker gene set forth in one of the panels or in Table 2a, Table 2b, Table 9 or Table 10 is measured. Often, such measurements may be performed using immunoassays. Protein expression level is determined using a breast tumor sample obtained from the patient.

A general overview of the applicable technology can be found in Harlow & Lane, Antibodies: A Laboratory Manual (1988) and Harlow & Lane, Using Antibodies (1999). Methods of producing polyclonal and monoclonal antibodies that react specifically with an allelic variant are known to those of skill in the art (see, e.g., Coligan, Current Protocols in Immunology (1991); Harlow & Lane, supra; Goding, Monoclonal Antibodies: Principles and Practice (2d ed. 1986); and Kohler & Milstein, Nature 256:495-497 (1975)). Such techniques include antibody preparation by selection of antibodies from libraries of recombinant antibodies in phage or similar vectors, as well as preparation of polyclonal and monoclonal antibodies by immunizing rabbits or mice (see, e.g., Huse et al., Science 246:1275-1281 (1989); Ward et al., Nature 341:544-546 (1989)).

Commonly used assays include noncompetitive assays, e.g., sandwich assays, and competitive assays. Typically, an assay such as an ELISA assay can be used. The amount of the polypeptide variant can be determined by performing quantitative analyses.

Other detection techniques, e.g., mass spectrometry techniques such as MALDI, may be used to directly detect the presence of proteins correlated with treatment outcomes.

As indicated above, evaluation of protein expression levels may additionally include determining the levels of protein expression of control genes, e.g., of one or more genes identified in Table 9 or 10.

In various embodiments, the present methods and gene detection may be carried out with or on a system incorporating computer and/or software elements configured for performing logic operations and calculations, input/output operations, machine communications, detection of gene or protein expressions levels and analysis of the measured levels and/or the like. Such system may also be used to generate a report, determinations of the total expression levels measured, the comparison with any reference levels, and calculation of the median levels of gene and gene product expression levels. It will be appreciated by one of skill in the art that various modifications are anticipated by the present embodiments.

In a further aspect, the invention provides diagnostic devices and kits for identifying gene expression products that are associated with prognosis for In some embodiments, a diagnostic device comprises probes to detect at least 5, 6, 7, 8, 9, 10, 11, 12, 13, 14, 15, 20, 25, 30, 36, 40, or including all 46 gene expression products of the genes in the described gene panels or the genes in Tables 9 or 10. In some embodiments, the present invention provides oligonucleotide probes attached to a solid support, such as an array slide or chip, e.g., as described in DNA Microarrays: A Molecular Cloning Manual, 2003, Eds. Bowtell and Sambrook, Cold Spring Harbor Laboratory Press. Construction of such devices are well known in the art, for example as described in US patents and patent Publications U.S. Pat. No. 5,837,832; PCT application WO95/11995; U.S. Pat. No. 5,807,522; U.S. Pat. Nos. 7,157,229, 7,083,975, 6,444,175, 6,375,903, 6,315,958, 6,295,153, and 5,143,854, 2007/0037274, 2007/0140906, 2004/0126757, 2004/0110212, 2004/0110211, 2003/0143550, 2003/0003032, and 2002/0041420. Nucleic acid arrays are also reviewed in the following references: Biotechnol Annu Rev 8:85-101 (2002); Sosnowski et al, Psychiatr Genet 12(4):181-92 (Dec. 2002); Heller, Annu Rev Biomed Eng 4: 129-53 (2002); Kolchinsky et al, Hum. Mutat 19(4):343-60 (April 2002); and McGail et al, Adv Biochem Eng Biotechnol 77:21-42 (2002).

An array can be composed of a large number of unique, single-stranded polynucleotides, usually either synthetic antisense polynucleotides or fragments of cDNAs, fixed to a solid support. Typical polynucleotides are preferably about 6-60 nucleotides in length, more preferably about 15-30 nucleotides in length, and most preferably about 18-25 nucleotides in length. For certain types of arrays or other detection kits/systems, it may be preferable to use oligonucleotides that are only about 7-20 nucleotides in length. In other types of arrays, such as arrays used in conjunction with chemiluminescent detection technology, preferred probe lengths can be, for example, about 15-80 nucleotides in length, preferably about 50-70 nucleotides in length, more preferably about 55-65 nucleotides in length, and most preferably about 60 nucleotides in length.

A person skilled in the art will recognize that, based on the known sequence information, detection reagents can be developed and used to assay any gene expression product set forth in Table 1 or Table 2 (or in some embodiments Table 3) and that such detection reagents can be incorporated into a kit. The term “kit” as used herein in the context of biomarker detection reagents, are intended to refer to such things as combinations of multiple biomarker detection reagents, or one or more biomarker detection reagents in combination with one or more other types of elements or components (e.g., other types of biochemical reagents, containers, packages such as packaging intended for commercial sale, substrates to which biomarker detection reagents are attached, electronic hardware components, etc.). Accordingly, the present invention further provides biomarker detection kits and systems, including but not limited to, packaged probe and primer sets (e.g., TaqMan probe/primer sets), arrays/microarrays of nucleic acid molecules where the arrays/microarrays comprise probes to detect the level of biomarker transcript, and beads that contain one or more probes, primers, or other detection reagents for detecting one or more biomarkers of the present invention. The kits can optionally include various electronic hardware components; for example, arrays (“DNA chips”) and microfluidic systems (“lab-on-a-chip” systems) provided by various manufacturers typically comprise hardware components. Other kits (e.g., probe/primer sets) may not include electronic hardware components, but may be comprised of, for example, one or more biomarker detection reagents (along with, optionally, other biochemical reagents) packaged in one or more containers.

In some embodiments, a biomarker detection kit typically contains one or more detection reagents and other components (e.g. a buffer, enzymes such as DNA polymerases) necessary to carry out an assay or reaction, such as amplification for detecting the level of biomarker transcript. A kit may further contain means for determining the amount of a target nucleic acid, and means for comparing the amount with a standard, and can comprise instructions for using the kit to detect the biomarker nucleic acid molecule of interest. In one embodiment of the present invention, kits are provided which contain the necessary reagents to carry out one or more assays to detect one or more biomarkers disclosed herein. In one embodiment of the present invention, biomarker detection kits/systems are in the form of nucleic acid arrays, or compartmentalized kits, including microfluidic/lab-on-a-chip systems.

Biomarker detection kits/systems may contain, for example, one or more probes, or pairs or sets of probes, that hybridize to a nucleic acid molecule encoded by a gene set forth in any of the gene panels described herein or in Table 9 or Table 10. In some embodiments, the presence of more than one biomarker can be simultaneously evaluated in an assay. For example, in some embodiments probes or probe sets to different biomarkers are immobilized as arrays or on beads. For example, the same substrate can comprise biomarkers probes for detecting at least 5, 6, 7, 8, 9, 10, 11, 12, 13, 14, 15, 20, 25, 30, 36, 40, 46 or including all genes or more of the biomarkers set forth herein or in Tables 2a, 2b, 9, 10 or 11. Thus, in some embodiments, the kit may comprise probes to detect a subset, multiple panels or all the panels described herein.

Using such arrays or other kits/systems, the present invention provides methods of identifying the biomarkers described herein in a test sample. Such methods typically involve incubating a test sample of nucleic acids obtained from a patient biopsy with an array comprising one or more probes that selectively hybridizes to a nucleic acid encoded by a gene set forth in one of the gene panels described herein or in Tables 2a, 2b, 9, 10 or 11. Such an array may additionally comprise probes to one or more reference genes. Conditions for incubating a biomarker detection reagent (or a kit/system that employs one or more such biomarker detection reagents) with a test sample vary. Incubation conditions depend on such factors as the format employed in the assay, the detection methods employed, and the type and nature of the detection reagents used in the assay. One skilled in the art will recognize that any one of the commonly available hybridization, amplification and array assay formats can readily be adapted to detect a biomarker set forth herein or in Tables 2a, 2b, 9, 10 or 11.

A biomarker detection kit of the present invention may include components that are used to prepare nucleic acids from a test sample for the subsequent amplification and/or detection of a biomarker nucleic acid molecule.

### Low-Dose Ionizing Radiation Induced Cancer Risk Gene Panels

Herein in one embodiment, we describe a systems biology approach to examine low dose ionizing radiation-induced genomic instability and expression responses (transcriptome with in situ protein analyses) in radiation sensitive and resistant strains, with the purpose of identifying candidate mechanisms of genetic susceptibility for low dose ionizing radiation-induced tissue damage and cancer risks. By the term “LD” it is meant to collectively refer to low dose ionizing radiation and low dose ionizing radiation-induced. Radiation-induced genomic instability is a hallmark of cancer, with strong evidence that it can be induced by high dose exposures. Using a sensitive flow method for detecting chromosomal damage in white blood cells, we demonstrate that high-dose exposure induces persistent genomic instability, but only in the cancer-sensitive BALB/c mice (not in C57BL/6). In contrast, LD exposure does not induce persistent genomic instability in either strain, even though BALB/c mice are more susceptible to LD-induced cancer.

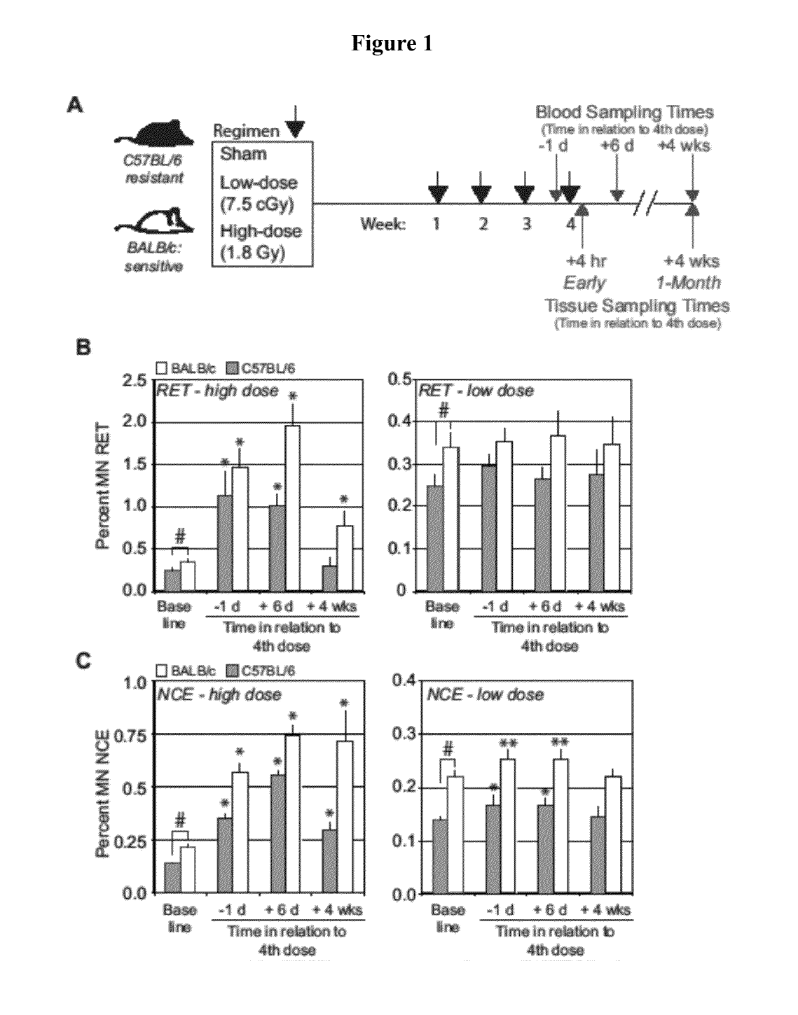

We then launched a system search for molecular mechanisms that might explain the strain differences in breast susceptibility to LD exposure using transcript profiling (Lowe X R, Bhattacharya S, Marchetti F, Wyrobek A J (2009) Early brain response to low-dose radiation exposure involves molecular networks and pathways associated with cognitive functions, advanced aging and Alzheimer's disease. Radiat Res 171: 53-6; Nguyen D H, Oketch-Rabah H A, Illa-Bochaca I, Geyer F C, Reis-Filho J S, et al. (2011) Radiation Acts on the Microenvironment to Affect Breast Carcinogenesis by Distinct Mechanisms that Decrease Cancer Latency and Affect Tumor Type. Cancer Cell 19: 640-651), since previously we found recurrent expression changes in cell lines from unrelated individuals after doses as low as 1 cGy (Wyrobek A J, Manohar C F, Krishnan V V, Nelson D O, Furtado M R, et al. (2011) Low dose radiation response curves, networks and pathways in human lymphoblastoid cells exposed from 1 to 10 cGy of acute gamma radiation. Mutat Res 722: 119-130). We investigated three exposure scenarios (FIG. 1A): (1) low dose (LD) group—four weekly doses of 7.5 cGy, (2) high dose (HD) group—four weekly doses of 1.8 Gy, (3) unexposed group—four weekly sham exposures. We analyzed expression profiles to identify expression signatures associated with biological functions that might explain the differential LD cancer susceptibility between these strains. We then tested LD susceptibility associated signatures in other murine and human knowledgebases (TGFβ-responsive, pubertal mammary development, human DCIS and breast cancer biomarkers, and disease free survival in human breast cancer patients) to understand their relevance to breast cancer (Loi S, Haibe-Kains B, Desmedt C, Lallemand F, Tutt A M, et al. (2007) Definition of clinically distinct molecular subtypes in estrogen receptor-positive breast carcinomas through genomic grade. J Clin Oncol 25: 1239-1246; Pawitan Y, Bjohle J, Amler L, Borg A L, Egyhazi S, et al. (2005) Gene expression profiling spares early breast cancer patients from adjuvant therapy: derived and validated in two population-based cohorts. Breast Cancer Res 7: R953-964; Abba M C, Lacunza E, Butti M, Aldaz C M (2010) Breast cancer biomarker discovery in the functional genomic age: a systematic review of 42 gene expression signatures. Biomark Insights 5: 103-118; McBryan J, Howlin J, Kenny P A, Shioda T, Martin F (2007) ERalpha-CITED1 co-regulated genes expressed during pubertal mammary gland development: implications for breast cancer prognosis. Oncogene 26: 6406-6419; Chen X L, Kapoun A M (2009) Heterogeneous activation of the TGFbeta pathway in glioblastomas identified by gene expression-based classification using TGFbeta-responsive genes. J Transl Med 7: 12).

Mouse models facilitate exploration of the biological and genetic features that influence risk of developing MG cancer as a result of LD exposure. The risk estimates for radiation-induced breast cancer, lung cancer and leukemia do not vary significantly between humans and mice, supporting the mouse as a reasonable surrogate model (Storer J B, Mitchell T J, Fry R J (1988) Extrapolation of the relative risk of radiogenic neoplasms across mouse strains and to man. Radiat Res 114: 331-35). We selected two inbred strains of mice that differ in their genetic susceptibility to radiation-induced MG cancer: BALB/c as more sensitive, and C57BL/6 strain as more resistant (Storer J B, Mitchell T J, Fry R J (1988) Extrapolation of the relative risk of radiogenic neoplasms across mouse strains and to man. Radiat Res 114: 331-35). BALB/c mice carry two DNA-PKcs polymorphisms with reduced protein expression, reduced catalytic activity and defective non-homologous-end-joining (NHEJ) of double strand breaks (Yu Y, Okayasu R, Weil M M, Silver A, McCarthy M, et al. (2001) Elevated breast cancer risk in irradiated BALB/c mice associates with unique functional polymorphism of the Prkdc (DNA-dependent protein kinase catalytic subunit) gene. Cancer Res 61: 1820-1824). But, as we will describe herein, BALB/c and C57BL/6 also vary in RNA processing and stress response functions (including other DNA repair genes) that may contribute to their genetic differences in radiation sensitivity.

We tested the hypothesis that genetic variation in baseline expression (i.e., expression levels before radiation exposure) and in responses to LD exposures can be used to identify tissue functions that determine susceptibility to LD-induced MG cancer and tissue functions that determine individual variation for better or poorer survival among breast cancer patients. We identified several tissue functions and two transcriptional signatures that are associated with susceptibility to LD-induced cancer in mice and with poor survival in breast cancer patients. This research lays the foundation for a new systems-biology approach for identifying the mechanisms of LD radiation-induced breast cancer, and suggests a new strategy to identify genetic features that predispose/protect individuals from risk of LD radiation-induced breast cancer.

Thus, herein are described several gene expression signatures that may be used to identify women at increased risk for low-dose radiation induced breast cancer. For example, at airports, women known to be at increased risk might benefit from avoiding (declining) the use of airport security scanners. In the medical field, women at increased risk might consider alternative diagnostic imaging modalities that do not use X-rays. Women undergoing radiotherapy treatment (high targeted doses of radiation) frequently are exposed to low-doses of radiation surrounding the target area. Additional shielding might be beneficial for women sensitive to low-dose radiation. In many instances knowing where a patient is on the spectrum of radiation sensitivity allows the patient and health care professionals to make an informed decision taking risk to benefit ratio into account.

Thus, in one embodiment, a panel is provided comprising nine biomarkers for prediction of disease free survival. In one embodiment, the signature for disease free survival is the group of nine genes comprising: BMP2K, CBX7, CLASP2, PRDX2, PRDX3, RAB6B, RPS6, RUNX1 and SLC15A2. Of this group of nine genes, four are characterized as tumor suppressor genes. The group of four tumor suppressor genes comprising: RUNX1, CBX7, PRDX2 and PRDX3. Lower expression in a patient results in a higher predicted probability of disease free survival. In some embodiments, the method for identifying a cancer patient with a higher probability of disease free survival, comprising: (a) measuring the amplification or expression levels of the group of genes encoding BMP2K, CBX7, CLASP2, PRDX2, PRDX3, RAB6B, RPS6, RUNX1 and SLC15A2 in a sample from the patient; and (b) comparing the sum of the amplification or expression level of the genes from the patient with median sum of amplification or expression level of the genes in a normal tissue sample or a reference expression level, wherein below-median expression indicates the patient with a higher probability of disease free survival.

BMP2K (BMP2 inducible kinase; Gene ID: 55589) is the human homolog of mouse BMP-2-inducible kinase. Bone morphogenic proteins (BMPs) play a key role in skeletal development and patterning. Expression of the mouse gene is increased during BMP-2 induced differentiation and the gene product is a putative serine/threonine protein kinase containing a nuclear localization signal. Therefore, the protein encoded by this human homolog is thought to be a protein kinase with a putative regulatory role in attenuating the program of osteoblast differentiation.

The expression level of a gene encoding BMP2K can also be measured by using or detecting the human nucleotide sequence, or a fragment thereof, of GenBank Accession No. NM_{—}017593.3 GI:38787903, Homo sapiens BMP2 inducible kinase (BMP2K), transcript variant 2, mRNA, which is expressed as BMP-2-inducible protein kinase isoform b [Homo sapiens], GenBank Accession No. NP_{—}060063.2 GI:38787904 or GenBank Accession No. NM_{—}198892.1 GI:38787934, Homo sapiens BMP2 inducible kinase (BMP2K), transcript variant 1, mRNA, which is expressed as BMP-2-inducible protein kinase isoform a [Homo sapiens], GenBank Accession No. NP_{—}942595.1 GI:38787935, the GenBank Accession and Gene information hereby incorporated by reference.

CBX7 (chromobox homolog 7, Gene ID: 23492) is a component of a Polycomb group (PcG) multiprotein PRC1-like complex, a complex class required to maintain the transcriptionally repressive state of many genes, including Hox genes, throughout development. PcG PRC1 complex acts via chromatin remodeling and modification of histones; it mediates monoubiquitination of histone H2A ‘Lys-119’, rendering chromatin heritably changed in its expressibility. Promotes histone H3 trimethylation at ‘Lys-9’ (H3K9me3). Binds to trimethylated lysine residues in histones, and possibly also other proteins. Regulator of cellular lifespan by maintaining the repression of CDKN2A, but not by inducing telomerase activity.

The expression level of a gene encoding CBX7 can also be measured by using or detecting the human nucleotide sequence, or a fragment thereof, of GenBank Accession No. NM_{—}175709.3 GI:156071505, Homo sapiens chromobox homolog 7 (CBX7), mRNA, which is expressed as chromobox protein homolog 7 [Homo sapiens], GenBank Accession No. NP_{—}783640.1 GI:28372505, the GenBank Accession and Gene information hereby incorporated by reference.

CLASP2 (cytoplasmic linker associated protein 2; Gene ID: 23122) is microtubule plus-end tracking protein that promotes the stabilization of dynamic microtubules. Involved in the nucleation of noncentrosomal microtubules originating from the trans-Golgi network (TGN). Required for the polarization of the cytoplasmic microtubule arrays in migrating cells towards the leading edge of the cell. May act at the cell cortex to enhance the frequency of rescue of depolymerizing microtubules by attaching their plus-ends to cortical platforms composed of ERC1 and PHLDB2. This cortical microtubule stabilizing activity is regulated at least in part by phosphatidylinositol 3-kinase signaling. Also performs a similar stabilizing function at the kinetochore which is essential for the bipolar alignment of chromosomes on the mitotic spindle. Acts as a mediator of ERBB2-dependent stabilization of microtubules at the cell cortex.

The expression level of a gene encoding BMP2K can also be measured by using or detecting the human nucleotide sequence, or a fragment thereof, of GenBank Accession No. NM_{—}001207044.1 GI:333440450, Homo sapiens cytoplasmic linker associated protein 2 (CLASP2), transcript variant 2, mRNA, which is expressed as CLIP-associating protein 2 isoform 2 [Homo sapiens], GenBank Accession No. NP_{—}001193973.1 GI:333440451 or GenBank Accession No. NM_{—}015097.2 GI:333440448, Homo sapiens cytoplasmic linker associated protein 2 (CLASP2), transcript variant 1, mRNA, which is expressed as CLIP-associating protein 2 isoform 1 [Homo sapiens], GenBank Accession No. NP_{—}055912.2 GI:333440449, the GenBank Accession and Gene information hereby incorporated by reference.

PRDX2 (peroxiredoxin 2; Gene ID: 7001) is member of the peroxiredoxin family of antioxidant enzymes, which reduce hydrogen peroxide and alkyl hydroperoxides. The encoded protein may play an antioxidant protective role in cells, and may contribute to the antiviral activity of CD8(+) T-cells. This protein may have a proliferative effect and play a role in cancer development or progression.

The expression level of a gene encoding PRDX2 can also be measured by using or detecting the human nucleotide sequence, or a fragment thereof, of GenBank Accession No. NM_{—}005809.4 GI:33188450, Homo sapiens peroxiredoxin 2 (PRDX2), nuclear gene encoding mitochondrial protein, transcript variant 1, mRNA, which is expressed as peroxiredoxin-2 isoform a [Homo sapiens], GenBank Accession No. NP_{—}005800.3 GI:32189392 or GenBank Accession No. NM_{—}181738.1 GI:33188453, Homo sapiens peroxiredoxin 2 (PRDX2), nuclear gene encoding mitochondrial protein, transcript variant 3, mRNA, which is expressed as peroxiredoxin-2 isoform c [Homo sapiens], GenBank Accession No. NP_{—}859428.1 GI:33188454, the GenBank Accession and Gene information hereby incorporated by reference.

PRDX3 (peroxiredoxin 3; Gene ID: 10935) is a gene that encodes a protein with antioxidant function and is localized in the mitochondrion. This gene shows significant nucleotide sequence similarity to the gene coding for the C22 subunit of Salmonella typhimurium alkylhydroperoxide reductase. Expression of this gene product in E. coli deficient in the C22-subunit gene rescued resistance of the bacteria to alkylhydroperoxide. The human and mouse genes are highly conserved, and they map to the regions syntenic between mouse and human chromosomes. Sequence comparisons with recently cloned mammalian homologues suggest that these genes consist of a family that is responsible for regulation of cellular proliferation, differentiation, and antioxidant functions.

The expression level of a gene encoding PRDX3 can also be measured by using or detecting the human nucleotide sequence, or a fragment thereof, of GenBank Accession No. NM_{—}006793.2 GI:32483378, Homo sapiens peroxiredoxin 3 (PRDX3), nuclear gene encoding mitochondrial protein, transcript variant 1, mRNA, which is expressed as thioredoxin-dependent peroxide reductase, mitochondrial isoform a precursor [Homo sapiens], GenBank Accession No. NP_{—}006784.1 GI:5802974 or GenBank Accession No. NM_{—}014098.2 GI:32483376, Homo sapiens peroxiredoxin 3 (PRDX3), nuclear gene encoding mitochondrial protein, transcript variant 2, mRNA, which is expressed as thioredoxin-dependent peroxide reductase, mitochondrial isoform b [Homo sapiens], GenBank Accession No. NP_{—}054817.2 GI:32483377, the GenBank Accession and Gene information hereby incorporated by reference.

RAB6B (RAB6B, member RAS oncogene family; RAB6B, member RAS oncogene family) has a role in retrograde membrane traffic at the level of the Golgi complex and may function in retrograde transport in neuronal cells.

The expression level of a gene encoding RAB6B can also be measured by using or detecting the human nucleotide sequence, or a fragment thereof, of GenBank Accession No. NM_{—}016577.3 GI:96975096, Homo sapiens RAB6B, member RAS oncogene family (RAB6B), mRNA, which is expressed as ras-related protein Rab-6B [Homo sapiens], GenBank Accession No. NP_{—}057661.3 GI:96975097, the GenBank Accession and Gene information hereby incorporated by reference.

RPS6 (ribosomal protein S6; Gene ID: 6194) is a gene that encodes a cytoplasmic ribosomal protein that is a component of the 40S subunit. The protein belongs to the S6E family of ribosomal proteins. It is the major substrate of protein kinases in the ribosome, with subsets of five C-terminal serine residues phosphorylated by different protein kinases. Phosphorylation is induced by a wide range of stimuli, including growth factors, tumor-promoting agents, and mitogens. Dephosphorylation occurs at growth arrest. The protein may contribute to the control of cell growth and proliferation through the selective translation of particular classes of mRNA.

The expression level of a gene encoding RPS6 can also be measured by using or detecting the human nucleotide sequence, or a fragment thereof, of GenBank Accession No. NM_{—}001010.2 GI:17158043, Homo sapiens ribosomal protein S6 (RPS6), mRNA, which is expressed as 40S ribosomal protein S6 [Homo sapiens], GenBank Accession No. NP_{—}001001.2 GI:17158044, the GenBank Accession and Gene information hereby incorporated by reference.

RUNX1 (runt-related transcription factor 1; Gene ID: 861) is a gene that encodes the alpha subunit of core binding factor (CBF) which is a heterodimeric transcription factor that binds to the core element of many enhancers and promoters and is involved in the development of normal hematopoiesis. Chromosomal translocations involving this gene are well-documented and have been associated with several types of leukemia.

The expression level of a gene encoding RUNX1 can also be measured by using or detecting the human nucleotide sequence, or a fragment thereof, of GenBank Accession No. NM_{—}001001890.2 GI:169790826, Homo sapiens runt-related transcription factor 1 (RUNX1), transcript variant 2, mRNA, which is expressed as runt-related transcription factor 1 isoform AML1b [Homo sapiens], GenBank Accession No. NP_{—}001001890.1 GI:49574546, GenBank Accession No. NM_{—}001122607.1 GI:169790836, Homo sapiens runt-related transcription factor 1 (RUNX1), transcript variant 3, mRNA, which is expressed as runt-related transcription factor 1 isoform AML1a [Homo sapiens], GenBank Accession No. NP_{—}001116079.1 GI:169790837 or GenBank Accession No. NM_{—}001754.4 GI:169790829, Homo sapiens runt-related transcription factor 1 (RUNX1), transcript variant 1, mRNA, which is expressed as runt-related transcription factor 1 isoform AML1c [Homo sapiens], GenBank Accession No. NP_{—}001745.2 GI:19923198, the GenBank Accession and Gene information hereby incorporated by reference.

SLC15A2 (solute carrier family 15 (H+/peptide transporter), member 2; Gene ID: 6565) is a proton-coupled peptide transporter expressed by the kidney that is responsible for the absorption of small peptides, as well as beta-lactam antibiotics and other peptide-like drugs, from the tubular filtrate.

The expression level of a gene encoding SLC15A2 can also be measured by using or detecting the human nucleotide sequence, or a fragment thereof, of GenBank Accession No. NM_{—}001145998.1 GI:226371747, Homo sapiens solute carrier family 15 (H+/peptide transporter) member 2 (SLC15A2), transcript variant 2, mRNA, which is expressed as solute carrier family 15 member 2 isoform b [Homo sapiens], GenBank Accession No. NP_{—}001139470.1 GI:226371748 or GenBank Accession No. NM_{—}021082.3 GI:226371745, Homo sapiens solute carrier family 15 (H+/peptide transporter), member 2 (SLC15A2), transcript variant 1, mRNA, which is expressed as solute carrier family 15 member 2 isoform a [Homo sapiens], GenBank Accession No. NP_{—}066568.3 GI:226371746, the GenBank Accession and Gene information hereby incorporated by reference.

In another embodiment, a panel is provided comprising 46 biomarkers for prediction of disease free survival. In one embodiment, the signature for disease free survival is the group of 46 genes comprising: MAGOHB, PAPOLA, PNPT1, POP4, PPIH, RBM39, SUPT16H, TXNL4A, EIF2S1, GNA13, GNB1, HLA-DRA, RAD23A, SMC6, ABCB10, ABCF1, BAT5, C17orf95, C19orf56, C5orf22, CAP1, CHCHD3, CHCHD4, CLDND1, DDX19A, DNAJC10, GADD45GIP1, GBP1, HLA-B, KIF5B, MCART1, MCM6, MTFR1, NRD1, PDK1, PDXDC1, PEBP1, PHF20, PI4K2B, PIGO, PPME1, SAPS3, SCAND1, SHC1, SNX6 and TM2D2.

MAGOHB (mago-nashi homolog B; Gene ID: 55110) is a gene involved in mRNA splicing and in the nonsense-mediated decay (NMD) pathway and belongs to the mago nashi family.

The expression level of a gene encoding MAGOHB can also be measured by using or detecting the human nucleotide sequence, or a fragment thereof, of GenBank Accession No. NM_{—}018048.3 GI:307078135, Homo sapiens mago-nashi homolog B (Drosophila) (MAGOHB), mRNA, which is expressed as protein mago nashi homolog 2 [Homo sapiens], GenBank Accession No. NP_{—}060518.1 GI:8922331, the GenBank Accession and Gene information hereby incorporated by reference.

PAPOLA (poly(A) polymerase alpha; Gene ID: 10914) is a gene that encodes a protein that belongs to the poly(A) polymerase family. It is required for the addition of adenosine residues for the creation of the 3′-poly(A) tail of mRNAs.

The expression level of a gene encoding PAPOLA can also be measured by using or detecting the human nucleotide sequence, or a fragment thereof, of GenBank Accession No. NM_{—}001252006.1 GI:354681990, Homo sapiens poly(A) polymerase alpha (PAPOLA), transcript variant 2, mRNA, which is expressed as poly(A) polymerase alpha isoform 2 [Homo sapiens], GenBank Accession No. NP_{—}001238935.1 GI:354681991, GenBank Accession No. NM_{—}001252007.1 GI:354681992, Homo sapiens poly(A) polymerase alpha (PAPOLA), transcript variant, which is expressed as poly(A) polymerase alpha isoform 3 [Homo sapiens], GenBank Accession No. NP_{—}001238936.1 GI:354681993 or GenBank Accession No. NM_{—}032632.4 GI:354681989, Homo sapiens poly(A) polymerase alpha (PAPOLA), transcript variant 1, mRNA, which is expressed as poly(A) polymerase alpha isoform 3 [Homo sapiens], GenBank Accession No. NP_{—}001238936.1 GI:354681993, the GenBank Accession and Gene information hereby incorporated by reference.

PNPT1 (polyribonucleotide nucleotidyltransferase 1; Gene ID: 87178) is a subunit of the exosome complex, which is involved in 3-prime-to-5-prime exoribonuclease activity for RNA processing and degradation.

The expression level of a gene encoding PNPT1 can also be measured by using or detecting the human nucleotide sequence, or a fragment thereof, of GenBank Accession No. NM_{—}033109.3 GI:188528627, Homo sapiens polyribonucleotide nucleotidyltransferase 1 (PNPT1), mRNA, which is expressed as polyribonucleotide nucleotidyltransferase 1, mitochondrial precursor [Homo sapiens], GenBank Accession No. NP_{—}149100.2 GI:188528628, the GenBank Accession and Gene information hereby incorporated by reference.

POP4 (processing of precursor 4, ribonuclease P/MRP subunit; Gene ID: 10775) is a gene that encodes one of the protein subunits of the small nucleolar ribonucleoprotein complexes: the endoribonuclease for mitochondrial RNA processing complex and the ribonuclease P complex. The encoded protein is localized to the nucleus and associates directly with the RNA component of these complexes. This protein is involved in processing of precursor RNAs.

The expression level of a gene encoding POP4 can also be measured by using or detecting the human nucleotide sequence, or a fragment thereof, of GenBank Accession No. NM_{—}006627.2 GI:225543171, Homo sapiens processing of precursor 4, ribonuclease P/MRP subunit (S. cerevisiae) (POP4), transcript variant 1, mRNA, which is expressed as ribonuclease P protein subunit p29 [Homo sapiens], GenBank Accession No. NP_{—}006618.1 GI:5729986 or GenBank Accession No. NR_{—}027368.1 GI:225543355, Homo sapiens processing of precursor 4, ribonuclease P/MRP subunit (S. cerevisiae) (POP4), transcript variant 2, non-coding RNA, the GenBank Accession and Gene information hereby incorporated by reference.

PPIH (peptidylprolyl isomerase H (cyclophilin H); Gene ID: 10465) is a gene that encodes a protein that is a member of the peptidyl-prolyl cis-trans isomerase (PPIase) family. PPlases catalyze the cis-trans isomerization of proline imidic peptide bonds in oligopeptides and accelerate the folding of proteins. This protein is a specific component of the complex that includes pre-mRNA processing factors PRPF3, PRPF4, and PRPF18, as well as U4/U5/U6 tri-snRNP. This protein has been shown to possess PPIase activity and may act as a protein chaperone that mediates the interactions between different proteins inside the spliceosome.

The expression level of a gene encoding PPIH can also be measured by using or detecting the human nucleotide sequence, or a fragment thereof, of GenBank Accession No. NM_{—}006347.3 GI:45439322, Homo sapiens peptidylprolyl isomerase H (cyclophilin H) (PPIH), mRNA, which is expressed as peptidyl-prolyl cis-trans isomerase H [Homo sapiens], GenBank Accession No. NP_{—}006338.1 GI:5454154, the GenBank Accession and Gene information hereby incorporated by reference.

RBM39 (RNA binding motif protein 39; Gene ID: 9584) is a gene that encodes a member of the U2AF65 family of proteins. The encoded protein is found in the nucleus, where it co-localizes with core spliceosomal proteins. It has been shown to play a role in both steroid hormone receptor-mediated transcription and alternative splicing, and it is also a transcriptional coregulator of the viral oncoprotein v-Rel.

The expression level of a gene encoding RBM39 can also be measured by using or detecting the human nucleotide sequence, or a fragment thereof, of GenBank Accession No. NM_{—}001242599.1 GI:336176063, Homo sapiens RNA binding motif protein 39 (RBM39), transcript variant 3, mRNA, which is expressed as RNA-binding protein 39 isoform c [Homo sapiens], GenBank Accession No. NP_{—}001229528.1 GI:336176064, GenBank Accession No. NM_{—}001242600.1 GI:336176065, Homo sapiens RNA binding motif protein 39 (RBM39), transcript variant 4, mRNA, which is expressed as RNA-binding protein 39 isoform d [Homo sapiens], GenBank Accession No. NP_{—}001229529.1 GI:336176066, GenBank Accession No. NM_{—}004902.3 GI:336176062, Homo sapiens RNA binding motif protein 39 (RBM39), transcript variant 2, mRNA, which is expressed as RNA-binding protein 39 isoform b [Homo sapiens], GenBank Accession No. NP_{—}004893.1 GI:4757926, GenBank Accession No. NM_{—}184234.2 GI:336176061, Homo sapiens RNA binding motif protein 39 (RBM39), transcript variant 1, mRNA, which is expressed as RNA-binding protein 39 isoform a [Homo sapiens], GenBank Accession No. NP_{—}909122.1 GI:35493811, GenBank Accession No. NR_{—}040722 NM_{—}001242601, Homo sapiens RNA binding motif protein 39 (RBM39), transcript variant 5, non-coding RNA, GenBank Accession No. NP_{—}001238936.1 GI:354681993, GenBank Accession No. NR_{—}040723.1 GI:342837694, Homo sapiens RNA binding motif protein 39 (RBM39), transcript variant 6, non-coding RNA or GenBank Accession No. NR_{—}040724 NM_{—}001242603, Homo sapiens RNA binding motif protein 39 (RBM39), transcript variant 7, non-coding RNA, the GenBank Accession and Gene information hereby incorporated by reference.

SUPT16H (suppressor of Ty 16 homolog; Gene ID: 11198) is a gene that encodes a transcription accessory factor subunit that facilitates transcription of DNA that is packaged into chromatin. FACT (facilitates chromatin transcription), interacts specifically with histones H2A/H2B to effect nucleosome disassembly and transcription elongation. FACT is composed of an 80 kDa subunit and a 140 kDa subunit; this gene encodes the 140 kDa subunit.

The expression level of a gene encoding SUPT16H can also be measured by using or detecting the human nucleotide sequence, or a fragment thereof, of GenBank Accession No. NM_{—}007192.3 GI:223890260, Homo sapiens suppressor of Ty 16 homolog (S. cerevisiae) (SUPT16H), mRNA, which is expressed as FACT complex subunit SPT16 [Homo sapiens], GenBank Accession No. NP_{—}009123.1 GI:6005757, the GenBank Accession and Gene information hereby incorporated by reference.

TXNL4A (thioredoxin-like 4A; Gene ID: 10907) is a gene that encodes a protein which plays an essential role in pre-mRNA splicing and has been shown to interact with PQBP1.

The expression level of a gene encoding TXNL4A can also be measured by using or detecting the human nucleotide sequence, or a fragment thereof, of GenBank Accession No. NM_{—}006701.2 GI:20070233, Homo sapiens thioredoxin-like 4A (TXNL4A), mRNA, which is expressed as thioredoxin-like protein 4A [Homo sapiens], GenBank Accession No. NP_{—}006692.1 GI:5729802, the GenBank Accession and Gene information hereby incorporated by reference.

EIF2S1 (eukaryotic translation initiation factor 2, subunit 1 alpha, 35 kDa; Gene ID: 1965) is the gene that encodes the alpha subunit of the translation initiation factor eIF2 complex which catalyzes the first regulated step of protein synthesis initiation, promoting the biding of the initiator tRNA to 40S ribosomal subunits. Binding occurs as a ternary complex of methionyl-tRNA, eIF2, and GTP. eIF2 is composed of 3 nonidentical subunits, alpha (36 kD, this article), beta (38 kD), and gamma (52 kD). The rate of formation of the ternary complex is modulated by the phosphorylation state of eIF2-alpha.

The expression level of a gene encoding EIF2S1 can also be measured by using or detecting the human nucleotide sequence, or a fragment thereof, of GenBank Accession No. NM_{—}004094.4 GI:77404353, Homo sapiens eukaryotic translation initiation factor 2, subunit 1 alpha, 35 kDa (EIF2S1), mRNA, which is expressed as eukaryotic translation initiation factor 2 subunit 1 [Homo sapiens], GenBank Accession No. NP_{—}004085.1 GI:4758256, the GenBank Accession and Gene information hereby incorporated by reference.

GNA13 (guanine nucleotide binding protein (G protein), alpha 13; Gene ID: 10672) is a guanine nucleotide-binding protein (G protein) that serves as a modulator or transducer in various transmembrane signaling systems.

The expression level of a gene encoding GNA13 can also be measured by using or detecting the human nucleotide sequence, or a fragment thereof, of GenBank Accession No. NM_{—}006572.4 GI:215820623, Homo sapiens guanine nucleotide binding protein (G protein), alpha 13 (GNA13), mRNA, which is expressed as guanine nucleotide-binding protein subunit alpha-13 [Homo sapiens], GenBank Accession No. NP_{—}006563.2 GI:24111250, the GenBank Accession and Gene information hereby incorporated by reference.

GNB1 (guanine nucleotide binding protein (G protein), beta polypeptide 1; Gene ID: 2782) is a gene that encodes a beta subunit of heterotrimeric guanine nucleotide-binding proteins (G proteins), which integrate signals between receptors and effector proteins. These subunits are encoded by families of related genes. Beta subunits are important regulators of alpha subunits, as well as of certain signal transduction receptors and effectors.

The expression level of a gene encoding GNB1 can also be measured by using or detecting the human nucleotide sequence, or a fragment thereof, of GenBank Accession No. NM_{—}002074.3 GI:324021693, Homo sapiens guanine nucleotide binding protein (G protein), beta polypeptide 1 (GNB1), mRNA, which is expressed as guanine nucleotide-binding protein G(I)/G(S)/G(T) subunit beta-1 [Homo sapiens], GenBank Accession No. NP_{—}002065.1 GI:11321585, the GenBank Accession and Gene information hereby incorporated by reference.

HLA-DRA (major histocompatibility complex, class II, DR alpha; Gene ID: 3122) is one of the HLA class II alpha chain paralogues. This class II molecule is a heterodimer consisting of an alpha and a beta chain, both anchored in the membrane. It plays a central role in the immune system by presenting peptides derived from extracellular proteins. Class II molecules are expressed in antigen presenting cells (APC: B lymphocytes, dendritic cells, macrophages).

The expression level of a gene encoding HLA-DRA can also be measured by using or detecting the human nucleotide sequence, or a fragment thereof, of GenBank Accession No. NM_{—}019111.4 GI:301171411, Homo sapiens major histocompatibility complex, class II, DR alpha (HLA-DRA), mRNA, which is expressed as HLA class II histocompatibility antigen, DR alpha chain precursor[Homo sapiens], GenBank Accession No. NP_{—}061984.2 GI:52426774, the GenBank Accession and Gene information hereby incorporated by reference.

RAD23A (RAD23 homolog A; Gene ID: 5886) is a gene that encodes a protein that is one of two human homologs of Saccharomyces cerevisiae Rad23, a protein involved in nucleotide excision repair. Proteins in this family have a modular domain structure consisting of an ubiquitin-like domain (UbL), ubiquitin-associated domain 1 (UbA1), XPC-binding domain and UbA2. The protein encoded by this gene plays an important role in nucleotide excision repair and also in delivery of polyubiquitinated proteins to the proteasome.

The expression level of a gene encoding RAD23A can also be measured by using or detecting the human nucleotide sequence, or a fragment thereof, of GenBank Accession No. NM_{—}001270362.1 GI:392996948, Homo sapiens RAD23 homolog A (S. cerevisiae) (RAD23A), transcript variant 2, mRNA, which is expressed as UV excision repair protein RAD23 homolog A isoform 2 [Homo sapiens], GenBank Accession No. NP_{—}001257291.1 GI:392996949, GenBank Accession No. NM_{—}001270363.1 GI:392996950, Homo sapiens RAD23 homolog A (S. cerevisiae) (RAD23A), transcript variant 3, mRNA, which is expressed as UV excision repair protein RAD23 homolog A isoform 3 [Homo sapiens], GenBank Accession No. NP_{—}001257292.1 GI:392996951, GenBank Accession No. NM_{—}005053.3 GI:392937549, Homo sapiens RAD23 homolog A (S. cerevisiae) (RAD23A), transcript variant 1, mRNA, which is expressed as UV excision repair protein RAD23 homolog A isoform 1 [Homo Sapiens], GenBank Accession No. NP_{—}005044.1 GI:4826964 or GenBank Accession No. NR_{—}072976.1 GI:392996952, Homo sapiens RAD23 homolog A (S. cerevisiae) (RAD23A), transcript variant 4, non-coding RNA, the GenBank Accession and Gene information hereby incorporated by reference.

SMC6 (structural maintenance of chromosomes 6; Gene ID: 79677) is a core component of the SMC5-SMC6 complex which is a complex involved in DNA double-strand breaks by homologous recombination. The complex may promote sister chromatid homologous recombination by recruiting the SMC1-SMC3 cohesin complex to double-strand breaks.

The expression level of a gene encoding SMC6 can also be measured by using or detecting the human nucleotide sequence, or a fragment thereof, of GenBank Accession No. NM_{—}001142286.1 GI:214010215, Homo sapiens structural maintenance of chromosomes 6 (SMC6), transcript variant 1, mRNA, which is expressed as structural maintenance of chromosomes protein 6 [Homo sapiens], GenBank Accession No. NP_{—}001135758.1 GI:214010216 or GenBank Accession No. NM_{—}024624.5 GI:214010214, Homo sapiens structural maintenance of chromosomes 6 (SMC6), transcript variant 2, mRNA, which is expressed as structural maintenance of chromosomes protein 6 [Homo sapiens], GenBank Accession No. NP_{—}078900.1 GI:13375848, the GenBank Accession and Gene information hereby incorporated by reference.

ABCB10 (ATP-binding cassette, sub-family B (MDR/TAP), member 10; Gene ID: 23456) is a gene that encodes a membrane-associated protein that is a member of the superfamily of ATP-binding cassette (ABC) transporters. ABC proteins transport various molecules across extra- and intra-cellular membranes. ABC genes are divided into seven distinct subfamilies (ABC1, MDR/TAP, MRP, ALD, OABP, GCN20, White). This protein is a member of the MDR/TAP subfamily. Members of the MDR/TAP subfamily are involved in multidrug resistance.

The expression level of a gene encoding ABCB10 can also be measured by using or detecting the human nucleotide sequence, or a fragment thereof, of GenBank Accession No. NM_{—}012089.2 GI:171184399, Homo sapiens ATP-binding cassette, sub-family B (MDR/TAP), member 10 (ABCB10), nuclear gene encoding mitochondrial protein, mRNA, which is expressed as ATP-binding cassette sub-family B member 10, mitochondrial [Homo Sapiens], GenBank Accession No. NP_{—}036221.2 GI:171184400, the GenBank Accession and Gene information hereby incorporated by reference.

ABCF1 (ATP-binding cassette, sub-family F (GCN20), member 1; Gene ID: 23) is a gene that encodes a protein that is a member of the superfamily of ATP-binding cassette (ABC) transporters. ABC proteins transport various molecules across extra- and intra-cellular membranes. ABC genes are divided into seven distinct subfamilies (ABC1, MDR/TAP, MRP, ALD, OABP, GCN20, White). This protein is a member of the GCN20 subfamily. Unlike other members of the superfamily, this protein lacks the transmembrane domains which are characteristic of most ABC transporters. This protein may be regulated by tumor necrosis factor-alpha and play a role in enhancement of protein synthesis and the inflammation process.

The expression level of a gene encoding ABCF1 can also be measured by using or detecting the human nucleotide sequence, or a fragment thereof, of GenBank Accession No. NM_{—}001025091.1 GI:69354670, Homo sapiens ATP-binding cassette, sub-family F (GCN20), member 1 (ABCF1), transcript variant 1, mRNA, which is expressed as ATP-binding cassette sub-family F member 1 isoform a [Homo sapiens], GenBank Accession No. NP_{—}001020262.1 GI:69354671 or GenBank Accession No. NM_{—}001090.2 GI:69354731, Homo sapiens ATP-binding cassette, sub-family F (GCN20), member 1 (ABCF1), transcript variant 2, mRNA, which is expressed as ATP-binding cassette sub-family F member 1 isoform b [Homo sapiens], GenBank Accession No. NP_{—}001081.1 GI:10947135, the GenBank Accession and Gene information hereby incorporated by reference.

BAT5 (also known as ABHD16A, abhydrolase domain containing 16A; Gene ID: 7920) is part of a cluster of genes, BAT1-BAT5, which have been localized in the vicinity of the genes for tumor necrosis factor alpha and tumor necrosis factor beta. These genes are all within the human major histocompatibility complex class III region. The protein encoded by this gene is thought to be involved in some aspects of immunity.

The expression level of a gene encoding BAT5 can also be measured by using or detecting the human nucleotide sequence, or a fragment thereof, of GenBank Accession No. NM_{—}001177515.1 GI:294660764, Homo sapiens abhydrolase domain containing 16A (ABHD16A), transcript variant 2, mRNA, which is expressed as abhydrolase domain-containing protein 16A isoform b [Homo sapiens], GenBank Accession No. NP_{—}001170986.1 GI:294660765, GenBank Accession No. NM_{—}021160.2 GI:294660762, Homo sapiens abhydrolase domain containing 16A (ABHD16A), transcript variant 1, mRNA, which is expressed as abhydrolase domain-containing protein 16A isoform a [Homo sapiens], GenBank Accession No. NP_{—}066983.1 GI:15100151, GenBank Accession No. NR_{—}033488.1 GI:294660763, Homo sapiens abhydrolase domain containing 16A (ABHD16A), transcript variant 3, non-coding RNA or GenBank Accession No. NR_{—}033489.1 GI:294660766, Homo sapiens abhydrolase domain containing 16A (ABHD16A), transcript variant 4, non-coding RNA, the GenBank Accession and Gene information hereby incorporated by reference.

C17orf95 (also known as METTL23, methyltransferase like 23; Gene ID: 124512) is a protein coding gene whose function is currently being studied and is a probable methyltransferase by similarity.

The expression level of a gene encoding C17orf95 can also be measured by using or detecting the human nucleotide sequence, or a fragment thereof, of GenBank Accession No. NM_{—}001080510.3 GI:332801030, Homo sapiens methyltransferase like 23 (METTL23), transcript variant 1, mRNA, which is expressed as methyltransferase-like protein 23 isoform 1 [Homo sapiens], GenBank Accession No. NP_{—}001073979.3 GI:332801031, GenBank Accession No. NM_{—}001206983.1 GI:332801032, Homo sapiens methyltransferase like 23 (METTL23), transcript variant 2, mRNA, which is expressed as methyltransferase-like protein 23 isoform 1 [Homo sapiens], GenBank Accession No. NP_{—}001193912.1 GI:332801033, GenBank Accession No. NM_{—}001206984.1 GI:332801034, Homo sapiens methyltransferase like 23 (METTL23), transcript variant 3, mRNA, which is expressed as methyltransferase-like protein 23 isoform 1 [Homo sapiens], GenBank Accession No. NP_{—}001193913.1 GI:332801035, GenBank Accession No. NM_{—}001206985.1 GI:332801036, Homo sapiens methyltransferase like 23 (METTL23), transcript variant 4, mRNA, which is expressed as methyltransferase-like protein 23 isoform 2 [Homo sapiens], GenBank Accession No. NP_{—}001193914.1 GI:332801037, GenBank Accession No. NM_{—}001206986.1 GI:332801038, Homo sapiens methyltransferase like 23 (METTL23), transcript variant 5, mRNA, which is expressed as methyltransferase-like protein 23 isoform 2 [Homo sapiens], GenBank Accession No. NP_{—}001193915.1 GI:332801039, GenBank Accession No. NM_{—}001206987.1 GI:332801040, Homo sapiens methyltransferase like 23 (METTL23), transcript variant 6, mRNA, which is expressed as methyltransferase-like protein 23 isoform 2 [Homo sapiens], GenBank Accession No. NP_{—}001193916.1 GI:332801041 or GenBank Accession No. NR_{—}038193.1 GI:332801042, Homo sapiens methyltransferase like 23 (METTL23), transcript variant 7, non-coding RNA, the GenBank Accession and Gene information hereby incorporated by reference.

C19orf56 (also known as WDR83OS, WD repeat domain 83 opposite strand; Gene ID: 51398) is a protein coding gene whose function is currently being studied.

The expression level of a gene encoding C19orf56 can also be measured by using or detecting the human nucleotide sequence, or a fragment thereof, of GenBank Accession No. NM_{—}016145.3 GI:194272193, Homo sapiens WD repeat domain 83 opposite strand (WDR83OS), mRNA, which is expressed as protein Asterix [Homo sapiens], NP_{—}057229.1 GI:7706665, the GenBank Accession and Gene information hereby incorporated by reference.

C5orf22 (chromosome 5 open reading frame 22, Gene ID: 55322) is a protein coding gene whose function is currently being studied.

The expression level of a gene encoding C5orf22 can also be measured by using or detecting the human nucleotide sequence, or a fragment thereof, of GenBank Accession No. NM_{—}018356.2 GI:170763485, Homo sapiens chromosome 5 open reading frame 22 (C5orf22), mRNA, which is expressed as UPF0489 protein C5orf22 [Homo sapiens], GenBank Accession No. NP_{—}060826.2 GI:170763486, the GenBank Accession and Gene information hereby incorporated by reference.

CAP1 (CAP, adenylate cyclase-associated protein 1 (yeast); Gene ID: 10487) is a gene that encodes a protein related to the S. cerevisiae CAP protein, which is involved in the cyclic AMP pathway. The human protein is able to interact with other molecules of the same protein, as well as with CAP2 and actin.

The expression level of a gene encoding CAP1 can also be measured by using or detecting the human nucleotide sequence, or a fragment thereof, of GenBank Accession No. NM_{—}001105530.1 GI:157649072, Homo sapiens CAP, adenylate cyclase-associated protein 1 (yeast) (CAP1), transcript variant 2, mRNA, which is expressed as adenylyl cyclase-associated protein 1 [Homo sapiens], GenBank Accession No. NP_{—}001099000.1 GI:157649073 or GenBank Accession No. NM_{—}006367.3 GI:157649071, Homo sapiens CAP, adenylate cyclase-associated protein 1 (yeast) (CAP1), transcript variant 1, mRNA, which is expressed as adenylyl cyclase-associated protein 1 [Homo sapiens], GenBank Accession No. NP_{—}006358.1 GI:5453595, the GenBank Accession and Gene information hereby incorporated by reference.

CHCHD3 (coiled-coil-helix-coiled-coil-helix domain containing 3, Gene ID: 54927) is a scaffolding protein that stabilizes protein complexes involved in maintaining mitochondrial crista architecture and protein import. Interacts with HSPA1A/HSPA1B and OPA1, preferentially with the soluble OPA1 form and also interacts with IMMT and SAMM50.

The expression level of a gene encoding CHCHD3 can also be measured by using or detecting the human nucleotide sequence, or a fragment thereof, of GenBank Accession No. NM_{—}017812.2 GI:142365297, Homo sapiens coiled-coil-helix-coiled-coil-helix domain containing 3 (CHCHD3), mRNA, which is expressed as coiled-coil-helix-coiled-coil-helix domain-containing protein 3, mitochondrial precursor [Homo sapiens]l, GenBank Accession No. NP_{—}060282.1 GI:8923390, the GenBank Accession and Gene information hereby incorporated by reference.

CHCHD4 (coiled-coil-helix-coiled-coil-helix domain containing 4; Gene ID: 131474) is a component of human mitochondria and belongs to a protein family whose members share 6 highly conserved cysteine residues constituting a -CXC-CX(9)C-CX(9)C- motif in the C terminus.

The expression level of a gene encoding CHCHD4 can also be measured by using or detecting the human nucleotide sequence, or a fragment thereof, of GenBank Accession No. NM_{—}001105530.1 GI:157649072, Homo sapiens CAP, adenylate cyclase-associated protein 1 (yeast) (CAP1), transcript variant 2, mRNA, which is expressed as adenylyl cyclase-associated protein 1 [Homo sapiens], GenBank Accession No. NP_{—}001099000.1 GI:157649073 or GenBank Accession No. NM_{—}006367.3 GI:157649071, Homo sapiens CAP, adenylate cyclase-associated protein 1 (yeast) (CAP1), transcript variant 1, mRNA, which is expressed as adenylyl cyclase-associated protein 1 [Homo sapiens], GenBank Accession No. NP_{—}006358.1 GI:5453595, the GenBank Accession and Gene information hereby incorporated by reference.

CLDND1 (claudin domain containing 1; Gene ID: 56650) is a protein coding gene whose function is currently being studied.

The expression level of a gene encoding CLDND1 can also be measured by using or detecting the human nucleotide sequence, or a fragment thereof, of GenBank Accession No. NM_{—}001040181.1 GI:93588613, Homo sapiens claudin domain containing 1 (CLDND1), transcript variant 1, mRNA, which is expressed as claudin domain-containing protein 1 isoform a [Homo sapiens], GenBank Accession No. NP_{—}001035271.1 GI:93588614, GenBank Accession No. NM_{—}001040182.1 GI:93588619, Homo sapiens claudin domain containing 1 (CLDND1), transcript variant 4, mRNA, which is expressed as claudin domain-containing protein 1 isoform b [Homo sapiens], GenBank Accession No. NP_{—}001035272.1 GI:93588620, GenBank Accession No. NM_{—}001040183.1 GI:93588623, Homo sapiens claudin domain containing 1 (CLDND1), transcript variant 3, mRNA, which is expressed as claudin domain-containing protein 1 isoform a [Homo sapiens], GenBank Accession No. NP_{—}001035273.1 GI:93588624, GenBank Accession No. NM_{—}001040199.1 GI:93588649, Homo sapiens claudin domain containing 1 (CLDND1), transcript variant 6, mRNA, which is expressed as claudin domain-containing protein 1 isoform a [Homo sapiens], GenBank Accession No. NP_{—}001035289.1 GI:93588650, GenBank Accession No. NM_{—}001040200.1 GI:93588656, Homo sapiens claudin domain containing 1 (CLDND1), transcript variant 7, mRNA, which is expressed as claudin domain-containing protein 1 isoform d [Homo sapiens], GenBank Accession No. NP_{—}001035290.1 GI:93588657 or GenBank Accession No. NM_{—}019895.2 GI:93588627, Homo sapiens claudin domain containing 1 (CLDND1), transcript variant 2, mRNA, which is expressed as claudin domain-containing protein 1 isoform a [Homo sapiens], GenBank Accession No. NP_{—}063948.1 GI:11096340, the GenBank Accession and Gene information hereby incorporated by reference.

DDX19A (DEAD (Asp-Glu-Ala-Asp) box polypeptide 19A; Gene ID: 55308) is a protein coding gene whose function is currently being studied and is likely an ATP-dependent RNA helicase involved in mRNA export from the nucleus by similarity.

The expression level of a gene encoding DDX19A can also be measured by using or detecting the human nucleotide sequence, or a fragment thereof, of GenBank Accession No. NM_{—}018332.3 GI:34147562, Homo sapiens DEAD (Asp-Glu-Ala-Asp) box polypeptide 19A (DDX19A), mRNA, which is expressed as ATP-dependent RNA helicase DDX19A [Homo sapiens], GenBank Accession No. NP_{—}060802.1 GI:8922886, the GenBank Accession and Gene information hereby incorporated by reference.

DNAJC10 (DnaJ (Hsp40) homolog, subfamily C, member 10; Gene ID: 54431) is an endoplasmic reticulum co-chaperone that may play a role in protein folding and translocation across the endoplasmic reticulum membrane and may act as a co-chaperone for HSPA5.

The expression level of a gene encoding DDX19A can also be measured by using or detecting the human nucleotide sequence, or a fragment thereof, of GenBank Accession No. NM_{—}018981.1 GI:24308126, Homo sapiens DnaJ (Hsp40) homolog, subfamily C, member 10 (DNAJC10), mRNA, which is expressed as dnaJ homolog subfamily C member 10 precursor [Homo sapiens], GenBank Accession No. NP_{—}061854.1 GI:24308127, the GenBank Accession and Gene information hereby incorporated by reference.

GADD45GIP1 (growth arrest and DNA-damage-inducible, gamma interacting protein 1; Gene ID: 90480) is a negative regulator of G1 to S cell cycle phase progression which acts by inhibiting cyclin-dependent kinases. Its inhibitory effects are additive with GADD45 proteins but occurs also in the absence of GADD45 proteins. It also acts as a repressor of the orphan nuclear receptor NR4A1 by inhibiting AB domain-mediated transcriptional activity and may be involved in the hormone-mediated regulation of NR4A1 transcriptional activity.

The expression level of a gene encoding GADD45GIP1 can also be measured by using or detecting the human nucleotide sequence, or a fragment thereof, of GenBank Accession No. NM_{—}052850.2 GI:31543414, Homo sapiens growth arrest and DNA-damage-inducible, gamma interacting protein 1 (GADD45GIP1), mRNA, which is expressed as growth arrest and DNA damage-inducible proteins-interacting protein 1 [Homo sapiens], GenBank Accession No. NP_{—}443082.2 GI:31543415, the GenBank Accession and Gene information hereby incorporated by reference.

GBP1 (guanylate binding protein 1, interferon-inducible; Gene ID: 2633) is encodes a guanylate binding protein where expression is induced by interferon. Guanylate binding proteins are characterized by their ability to specifically bind guanine nucleotides (GMP, GDP, and GTP) and are distinguished from the GTP-binding proteins by the presence of 2 binding motifs rather than 3.

The expression level of a gene encoding GBP1 can also be measured by using or detecting the human nucleotide sequence, or a fragment thereof, of GenBank Accession No. NM_{—}002053.2 GI:166706902, Homo sapiens guanylate binding protein 1, interferon-inducible (GBP1), mRNA, which is expressed as interferon-induced guanylate-binding protein 1 [Homo sapiens], GenBank Accession No. NP_{—}002044.2 GI:166706903, the GenBank Accession and Gene information hereby incorporated by reference.

HLA-B (major histocompatibility complex, class I, B; Gene ID: 3106) belongs to the HLA class I heavy chain paralogues. This class I molecule is a heterodimer consisting of a heavy chain and a light chain (beta-2 microglobulin). The heavy chain is anchored in the membrane. Class I molecules play a central role in the immune system by presenting peptides derived from the endoplasmic reticulum lumen. They are expressed in nearly all cells.

The expression level of a gene encoding HLA-B can also be measured by using or detecting the human nucleotide sequence, or a fragment thereof, of GenBank Accession No. NM_{—}005514.6 GI:170650640 Homo sapiens major histocompatibility complex, class I, B (HLA-B), mRNA, which is expressed as major histocompatibility complex, class I, B precursor [Homo sapiens], GenBank Accession No. NP_{—}005505.2 GI:17986001, the GenBank Accession and Gene information hereby incorporated by reference.

KIF5B (kinesin family member 5B; Gene ID: 3799) is a protein coding gene whose function is currently being studied and is likely a microtubule-dependent motor required for normal distribution of mitochondria and lysosomes by similarity.

The expression level of a gene encoding KIF5B can also be measured by using or detecting the human nucleotide sequence, or a fragment thereof, of GenBank Accession No. NM_{—}004521.2 GI:187761329m, Homo sapiens kinesin family member 5B (KIF5B), mRNA, which is expressed as kinesin-1 heavy chain [Homo sapiens], GenBank Accession No. NP_{—}004512.1 GI:4758648, the GenBank Accession and Gene information hereby incorporated by reference.

MCART1 (also known as SLC25A51, solute carrier family 25, member 51; Gene ID: 92014) is a protein coding gene whose function is currently being studied.

The expression level of a gene encoding MCART1 can also be measured by using or detecting the human nucleotide sequence, or a fragment thereof, of GenBank Accession No. NM_{—}033412.3 GI:219842355, Homo sapiens solute carrier family 25, member 51 (SLC25A51), nuclear gene encoding mitochondrial protein, transcript variant 1, mRNA, which is expressed as solute carrier family 25 member 51 [Homo sapiens], GenBank Accession No. NP_{—}219480.1 GI:15529972, GenBank Accession No. NR_{—}024872.1 GI:219842279, Homo sapiens solute carrier family 25, member 51 (SLC25A51), transcript variant 2, non-coding RNA or GenBank Accession No. NR_{—}024873.1 GI:219842280, Homo sapiens solute carrier family 25, member 51 (SLC25A51), transcript variant 3, non-coding RNA, the GenBank Accession and Gene information hereby incorporated by reference.

MCM6 (minichromosome maintenance complex component 6; Gene ID: 4175) is a gene that encodes a protein that is a highly conserved mini-chromosome maintenance protein (MCM) that is essential for the initiation of eukaryotic genome replication. The hexameric protein complex formed by the MCM proteins is a key component of the pre-replication complex (pre_RC) and may be involved in the formation of replication forks and in the recruitment of other DNA replication related proteins. The MCM complex consisting of this protein and MCM2, 4 and 7 proteins possesses DNA helicase activity, and may act as a DNA unwinding enzyme. The phosphorylation of the complex by CDC2 kinase reduces the helicase activity, suggesting a role in the regulation of DNA replication. Single nucleotide polymorphisms in the intron regions of this gene are associated with differential transcriptional activation of the promoter of the neighboring lactase gene and, thereby, influence lactose intolerance in early adulthood.

The expression level of a gene encoding MCM6 can also be measured by using or detecting the human nucleotide sequence, or a fragment thereof, of GenBank Accession No. NM_{—}005915.5 GI:386869284, Homo sapiens minichromosome maintenance complex component 6 (MCM6), mRNA, which is expressed as DNA replication licensing factor MCM6 [Homo sapiens], GenBank Accession No. NP_{—}005906.2 GI:7427519, the GenBank Accession and Gene information hereby incorporated by reference.

MTFR1 (mitochondrial fission regulator 1; Gene ID: 9650) is a gene that encodes a mitochondrial protein that is characterized by a poly-proline rich region. A chicken homolog of this protein promotes mitochondrial fission and the mouse homolog protects cells from oxidative stress.

The expression level of a gene encoding MTFR1 can also be measured by using or detecting the human nucleotide sequence, or a fragment thereof, of GenBank Accession No. NM_{—}001145838.1 GI:224994161, Homo sapiens mitochondrial fission regulator 1 (MTFR1), nuclear gene encoding mitochondrial protein, transcript variant 2, mRNA, which is expressed as mitochondrial fission regulator 1 isoform 2 [Homo sapiens], GenBank Accession No. NP_{—}001139310.1 GI:224994162, GenBank Accession No. NM_{—}001145839.1 GI:224994163, Homo sapiens mitochondrial fission regulator 1 (MTFR1), nuclear gene encoding mitochondrial protein, transcript variant 3, mRNA, which is expressed as mitochondrial fission regulator 1 isoform 3 [Homo sapiens], GenBank Accession No. NP_{—}001139311. GI:224994164 or GenBank Accession No. NM_{—}014637.3 GI:224994159, Homo sapiens mitochondrial fission regulator 1 (MTFR1), nuclear gene encoding mitochondrial protein, transcript variant 1, mRNA, which is expressed as mitochondrial fission regulator 1 isoform 1 [Homo sapiens], GenBank Accession No. NP_{—}055452.3 GI:224994160, the GenBank Accession and Gene information hereby incorporated by reference.

NRD1 (nardilysin (N-arginine dibasic convertase; Gene ID: 4898) is a gene that encodes a zinc-dependent endopeptidase that cleaves peptide substrates at the N-terminus of arginine residues in dibasic moieties and is a member of the peptidase M16 family. This protein interacts with heparin-binding EGF-like growth factor and plays a role in cell migration and proliferation.

The expression level of a gene encoding NRD1 can also be measured by using or detecting the human nucleotide sequence, or a fragment thereof, of GenBank Accession No. NM_{—}001101662.1 GI:156071451, Homo sapiens nardilysin (N-arginine dibasic convertase) (NRD1), transcript variant 2, mRNA, which is expressed as nardilysin isoform b precursor [Homo sapiens], GenBank Accession No. NP_{—}001095132.1 GI:156071452, GenBank NM_{—}001242361.1 GI:334358860, Homo sapiens nardilysin (N-arginine dibasic convertase) (NRD1), transcript variant 3, mRNA, which is expressed as nardilysin isoform c [Homo sapiens], GenBank Accession No. NP_{—}001229290. GI:334358861 or GenBank Accession NM_{—}002525.2 GI:156071449, Homo sapiens nardilysin (N-arginine dibasic convertase) (NRD1), transcript variant 1, mRNA, which is expressed as nardilysin isoform a precursor [Homo sapiens], GenBank Accession No. NP_{—}002516. GI:156071450, the GenBank Accession and Gene information hereby incorporated by reference.

PDK1 (pyruvate dehydrogenase kinase, isozyme 1; Gene ID: 5163) is a specific kinase that regulates the enzymatic activity of pyruvate dehydrogenase (PDH) by phosphorylation which results in inactivation of PDH. PDH is a mitochondrial multienzyme complex that catalyzes the oxidative decarboxylation of pyruvate and is one of the major enzymes responsible for the regulation of homeostasis of carbohydrate fuels in mammals.

The expression level of a gene encoding PDK1 can also be measured by using or detecting the human nucleotide sequence, or a fragment thereof, of GenBank Accession No. NM_{—}002610.3 GI:37595546, Homo sapiens pyruvate dehydrogenase kinase, isozyme 1 (PDK1), nuclear gene encoding mitochondrial protein, mRNA, which is expressed as pyruvate dehydrogenase kinase, isozyme 1 [Homo sapiens], GenBank Accession No. NP_{—}002601.1 GI:4505689, the GenBank Accession and Gene information hereby incorporated by reference.

PDXDC1 (pyridoxal-dependent decarboxylase domain containing 1; Gene ID: 23042) is a protein coding gene whose function is currently being studied.

The expression level of a gene encoding PDXDC1 can also be measured by using or detecting the human nucleotide sequence, or a fragment thereof, of GenBank Accession No. NM_{—}015027.2 GI:190341073, Homo sapiens pyridoxal-dependent decarboxylase domain containing 1 (PDXDC1), mRNA, which is expressed as pyridoxal-dependent decarboxylase domain-containing protein 1 [Homo sapiens], GenBank Accession No. NP_{—}055842 XP_{—}352159, the GenBank Accession and Gene information hereby incorporated by reference.

PEBP1 (phosphatidylethanolamine binding protein 1; Gene ID: 5037) affects various cellular processes, and is implicated in metastasis formation and Alzheimer's disease. Human PEBP1 has also been shown to inhibit the Raf/MEK/ERK pathway. It also binds ATP, opioids and phosphatidylethanolamine and has lower affinity for phosphatidylinositol and phosphatidylcholine. It also acts as a serine protease inhibitor which inhibits thrombin, neuropsin and chymotrypsin but not trypsin, tissue type plasminogen activator and elastase and inhibits the kinase activity of RAF1 by inhibiting its activation and by dissociating the RAF1/MEK complex and acting as a competitive inhibitor of MEK phosphorylation.

The expression level of a gene encoding PEBP1 can also be measured by using or detecting the human nucleotide sequence, or a fragment thereof, of GenBank Accession No. NM_{—}002567.2 GI:38016928, Homo sapiens phosphatidylethanolamine binding protein 1 (PEBP1), mRNA, which is expressed as phosphatidylethanolamine-binding protein 1 preproprotein [Homo sapiens], GenBank Accession No. NP_{—}002558.1 GI:4505621, the GenBank Accession and Gene information hereby incorporated by reference.

PHF20 (PHD finger protein 20; Gene ID: 51230) is PHF20 is a methyl lysine binding protein that is a component of the MOF histone acetyltransferase protein complex. It is not required for maintaining the global histone H4 ‘Lys-16’ acetylation (H4K16ac) levels or locus specific histone acetylation, but instead works downstream in transcriptional regulation of MOF target genes.

The expression level of a gene encoding PHF20 can also be measured by using or detecting the human nucleotide sequence, or a fragment thereof, of GenBank Accession No. NM_{—}016436.4 GI:110735447, Homo sapiens PHD finger protein 20 (PHF20), mRNA, which is expressed as PHD finger protein 20 [Homo sapiens], GenBank Accession No. NP_{—}057520.2 GI:18034775, the GenBank Accession and Gene information hereby incorporated by reference.

PI4K2B (phosphatidylinositol 4-kinase type 2 beta; Gene ID: 55300) is a gene that encodes a protein which phosphorylates phosphatidylinositol to generate phosphatidylinositol 4-phosphate (PIP), an immediate precursor of several important signaling and scaffolding molecules. PIP itself may also have direct functional and structural roles. PI4K2B is a primarily cytosolic PI4K that is recruited to membranes, where it stimulates phosphatidylinositol 4,5-bisphosphate synthesis

The expression level of a gene encoding PI4K2B can also be measured by using or detecting the human nucleotide sequence, or a fragment thereof, of GenBank Accession No. NM_{—}018323.3 GI:224591400, Homo sapiens phosphatidylinositol 4-kinase type 2 beta (PI4K2B), mRNA, which is expressed as phosphatidylinositol 4-kinase type 2-beta [Homo sapiens], GenBank Accession No. NP_{—}060793.2 GI:224591401, the GenBank Accession and Gene information hereby incorporated by reference.

PIGO (phosphatidylinositol glycan anchor biosynthesis, class 0, Gene ID: 84720) is a gene that encodes a protein that is involved in glycosylphosphatidylinositol (GPI)-anchor biosynthesis. The GPI-anchor is a glycolipid which contains three mannose molecules in its core backbone. The GPI-anchor is found on many blood cells and serves to anchor proteins to the cell surface. This protein is involved in the transfer of ethanolaminephosphate (EtNP) to the third mannose in GPI.

The expression level of a gene encoding PIGO can also be measured by using or detecting the human nucleotide sequence, or a fragment thereof, of GenBank Accession No. NM_{—}001201484.1 GI:319918881, Homo sapiens phosphatidylinositol glycan anchor biosynthesis, class O (PIGO), transcript variant 3, mRNA, which is expressed as GPI ethanolamine phosphate transferase 3 isoform 2 [Homo sapiens], GenBank Accession No. NP_{—}001188413.1 GI:319918882, GenBank NM_{—}032634.3 GI:319918879, Homo sapiens phosphatidylinositol glycan anchor biosynthesis, class O (PIGO), transcript variant 1, mRNA, which is expressed as GPI ethanolamine phosphate transferase 3 isoform 1 [Homo sapiens], GenBank Accession No. NP 116023.2 GI:23397648 or GenBank Accession NM_{—}152850.3 GI:319918880, Homo sapiens phosphatidylinositol glycan anchor biosynthesis, class O (PIGO), transcript variant 2, mRNA, which is expressed as GPI ethanolamine phosphate transferase 3 isoform 2 [Homo sapiens], GenBank Accession No. NP_{—}690577.2 GI:38045917, the GenBank Accession and Gene information hereby incorporated by reference.

PPME1 (protein phosphatase methylesterase 1, Gene ID: 51400) catalyzes the demethylation of the protein phosphatase-2A catalytic subunit.

The expression level of a gene encoding PPME1 can also be measured by using or detecting the human nucleotide sequence, or a fragment thereof, of GenBank Accession No. NM_{—}016147.1 GI:7706644, Homo sapiens protein phosphatase methylesterase 1 (PPME1), mRNA, which is expressed as protein phosphatase methylesterase 1 [Homo sapiens], GenBank Accession No. NP_{—}057231.1 GI:7706645, the GenBank Accession and Gene information hereby incorporated by reference.

SAPS3 (also known as PPP6R3, protein phosphatase 6, regulatory subunit 3, Gene ID: 55291) is a regulatory subunit for the protein phosphatase-6 catalytic subunit. Protein phosphatase regulatory subunits, such as SAPS3, modulate the activity of protein phosphatase catalytic subunits by restricting substrate specificity, recruiting substrates, and determining the intracellular localization of the holoenzyme.

The expression level of a gene encoding SAPS3 can also be measured by using or detecting the human nucleotide sequence, or a fragment thereof, of GenBank Accession No. NM_{—}001164160.1 GI:255918191, Homo sapiens protein phosphatase 6, regulatory subunit 3 (PPP6R3), transcript variant 4, mRNA, which is expressed as serine/threonine-protein phosphatase 6 regulatory subunit 3 isoform 4 [Homo sapiens], GenBank Accession No. NP_{—}001157632.1 GI:255918192, GenBank Accession No. NM_{—}001164161.1 GI:255918193, Homo sapiens protein phosphatase 6, regulatory subunit 3 (PPP6R3), transcript variant 6, mRNA, which is expressed as serine/threonine-protein phosphatase 6 regulatory subunit 3 isoform 6 [Homo sapiens], GenBank Accession No. NP_{—}001157633.1 GI:255918194, GenBank Accession No. NM_{—}001164162.1 GI:255918195, Homo sapiens protein phosphatase 6, regulatory subunit 3 (PPP6R3), transcript variant 1, mRNA, which is expressed as serine/threonine-protein phosphatase 6 regulatory subunit 3 isoform 1 [Homo sapiens], GenBank Accession No. NP_{—}001157634.1 GI:255918196, GenBank Accession No. NM_{—}001164163.1 GI:255918197, Homo sapiens protein phosphatase 6, regulatory subunit 3 (PPP6R3), transcript variant 2, mRNA, which is expressed as serine/threonine-protein phosphatase 6 regulatory subunit 3 isoform 2 [Homo sapiens], GenBank Accession No. NP_{—}001157635.1 GI:255918198, GenBank Accession No. NM_{—}001164164.1 GI:255918199, Homo sapiens protein phosphatase 6, regulatory subunit 3 (PPP6R3), transcript variant 3, mRNA, which is expressed as serine/threonine-protein phosphatase 6 regulatory subunit 3 isoform 3 [Homo sapiens], GenBank Accession No. NP_{—}001157636.1 GI:255918200 or GenBank Accession No. NM_{—}018312.4 GI:255918189, Homo sapiens protein phosphatase 6, regulatory subunit 3 (PPP6R3), transcript variant 5, mRNA, which is expressed as serine/threonine-protein phosphatase 6 regulatory subunit 3 isoform 5 [Homo sapiens], GenBank Accession No. NP_{—}060782.2 GI:13489083, the GenBank Accession and Gene information hereby incorporated by reference.

SCAND1 (SCAN domain containing 1, Gene ID: 51282) is a gene that encodes a SCAN box domain-containing protein. The SCAN domain is a highly conserved, leucine-rich motif of approximately 60 aa originally found within a subfamily of zinc finger proteins. This gene belongs to a family of genes that encode an isolated SCAN domain, but no zinc finger motif. This protein binds to and may regulate the function of the transcription factor myeloid zinc finger 1B.

The expression level of a gene encoding SCAND1 can also be measured by using or detecting the human nucleotide sequence, or a fragment thereof, of GenBank Accession No. NM_{—}016558.3 GI:319022228, Homo sapiens SCAN domain containing 1 (SCAND1), transcript variant 1, mRNA, which is expressed as SCAN domain-containing protein 1 isoform 1 [Homo sapiens], GenBank Accession No. NP_{—}057642.1 GI:7706089 or GenBank Accession No. NM_{—}033630.2 GI:319655724, Homo sapiens SCAN domain containing 1 (SCAND1), transcript variant 2, mRNA, which is expressed as SCAN domain-containing protein 1 isoform 2 [Homo sapiens], GenBank Accession No. NP_{—}361012.2 GI:319655725, the GenBank Accession and Gene information hereby incorporated by reference.

SHC1 (SHC (Src homology 2 domain containing) transforming protein 1, Gene ID: 6464) is a gene that encodes three main isoforms that differ in activities and subcellular location. While all three are adapter proteins are involved in the signal transduction pathways, the longest (p66Shc) may be involved in regulating life span and the effects of reactive oxygen species. The other two isoforms, p52Shc and p46Shc, link activated receptor tyrosine kinases to the Ras pathway by recruitment of the GRB2/SOS complex. p66Shc is not involved in Ras activation. Unlike the other two isoforms, p46Shc is targeted to the mitochondrial matrix.

The expression level of a gene encoding SHC1 can also be measured by using or detecting the human nucleotide sequence, or a fragment thereof, of GenBank Accession No. NM_{—}001130040.1 GI:194239663, Homo sapiens SHC (Src homology 2 domain containing) transforming protein 1 (SHC1), transcript variant 3, mRNA, which is expressed as SHC-transforming protein 1 isoform 3 [Homo sapiens], GenBank Accession No. NP_{—}001123512.1 GI:194239664, GenBank Accession No. NM_{—}001130041.1 GI:194239667, Homo sapiens SHC (Src homology 2 domain containing) transforming protein 1 (SHC1), transcript variant 4, mRNA, which is expressed as SHC-transforming protein 1 isoform 4 [Homo sapiens], GenBank Accession No. NP_{—}001123513.1 GI:194239668, GenBank Accession No. NM_{—}001202859.1 GI:322302754, Homo sapiens SHC (Src homology 2 domain containing) transforming protein 1 (SHC1), nuclear gene encoding mitochondrial protein, transcript variant 5, mRNA, which is expressed as SHC-transforming protein 1 isoform 5 precursor [Homo sapiens], GenBank Accession No. NP_{—}001189788.1 GI:322302755, GenBank Accession No. NM_{—}003029.4 GI:194239660, Homo sapiens SHC (Src homology 2 domain containing) transforming protein 1 (SHC1), transcript variant 2, mRNA, which is expressed as SHC-transforming protein 1 isoform 2 [Homo sapiens], GenBank Accession No. NP_{—}003020.2 GI:32261324 or GenBank Accession No. NM_{—}183001.4 GI:194239661, Homo sapiens SHC (Src homology 2 domain containing) transforming protein 1 (SHC1), transcript variant 1, mRNA, which is expressed as SHC-transforming protein 1 isoform 1 [Homo sapiens], GenBank Accession No. NP_{—}892113.4 GI:194239662, the GenBank Accession and Gene information hereby incorporated by reference.

SNX6 (sorting nexin 6, Gene ID: 58533) is a gene that encodes a member of the sorting nexin family. Members of this family contain a phox (PX) domain, which is a phosphoinositide binding domain, and are involved in intracellular trafficking. This protein associates with the long isoform of the leptin receptor, the transforming growth factor-beta family of receptor serine-threonine kinases, and with receptor tyrosine kinases for platelet-derived growth factor, insulin, and epidermal growth factor. This protein may form oligomeric complexes with family member proteins through interactions of both the PX domain and the coiled coil regions of the molecules. Translocation of this protein from the cytoplasm to the nucleus occurs after binding to proviral integration site 1 protein.

The expression level of a gene encoding SNX6 can also be measured by using or detecting the human nucleotide sequence, or a fragment thereof, of GenBank Accession No. NM_{—}021249.3 GI:88703042, Homo sapiens sorting nexin 6 (SNX6), transcript variant 1, mRNA, which is expressed as sorting nexin-6 isoform a [Homo sapiens], GenBank Accession No. NP_{—}067072.3 GI:88703043 or GenBank Accession No. NM_{—}152233.2 GI:88703040, Homo sapiens sorting nexin 6 (SNX6), transcript variant 2, mRNA, which is expressed as sorting nexin-6 isoform b [Homo sapiens], GenBank Accession No. NP_{—}689419.2 GI:88703041, the GenBank Accession and Gene information hereby incorporated by reference.

TM2D2 (TM2 domain containing 2, Gene ID: 83877) is a gene that encodes a protein that contains a structural module related to that of the seven transmembrane domain G protein-coupled receptor superfamily. This protein has sequence and structural similarities to the beta-amyloid binding protein (BBP), but, unlike BBP, it does not regulate a response to beta-amyloid peptide. This protein may have regulatory roles in cell death or proliferation signal cascades.

The expression level of a gene encoding TM2D2 can also be measured by using or detecting the human nucleotide sequence, or a fragment thereof, of GenBank Accession No. NM_{—}001024380.1 GI:66932894, Homo sapiens TM2 domain containing 2 (TM2D2), transcript variant 3, mRNA, which is expressed as TM2 domain-containing protein 2 isoform b [Homo sapiens], GenBank Accession No. NP_{—}001019551.1 GI:66932895, GenBank Accession No. NM_{—}001024381.1 GI:66932896, Homo sapiens TM2 domain containing 2 (TM2D2), transcript variant 4, mRNA, which is expressed as TM2 domain-containing protein 2 isoform b [Homo sapiens], GenBank Accession No. NP_{—}001019552.1 GI:66932897, GenBank Accession No. NM_{—}031940.3 GI:66932892, Homo sapiens TM2 domain containing 2 (TM2D2), transcript variant 2, mRNA, which is expressed as TM2 domain-containing protein 2 isoform b [Homo sapiens], GenBank Accession No. NP_{—}114146.3 GI:66932893 or GenBank Accession No. NM_{—}078473.2 GI:66932891, Homo sapiens TM2 domain containing 2 (TM2D2), transcript variant 1, mRNA, which is expressed as TM2 domain-containing protein 2 isoform a precursor [Homo sapiens], GenBank Accession No. NP_{—}510882.1 GI:17865797, the GenBank Accession and Gene information hereby incorporated by reference.

55 Biomarkers for Prediction of Disease Free Survival.

In one embodiment, the signature for disease free survival is the group of 55 genes comprising: MAGOHB, PAPOLA, PNPT1, POP4, PPIH, RBM39, RPS6, SUPT16H, TXNL4A, EIF2S1, GNA13, GNB1, HLA-DRA, PRDX2, PRDX3, RAD23A, RUNX1, SMC6, ABCB10, ABCF1, BAT5, BMP2K, C17orf95, C19orf56, C5orf22, CAP1, CBX7, CHCHD3, CHCHD4, CLASP2, CLDND1, DDX19A, DNAJC10, GADD45GIP1, GBP1, HLA-B, KIF5B, MCART1, MCM6, MTFR1, NRD1, PDK1, PDXDC1, PEBP1, PHF20, PI4K2B, PIGO, PPME1, RAB6B, SAPS3, SCAND1, SHC1, SLC15A2, SNX6 and TM2D2. Of this group of 55 genes, 9 are characterized as RNA processing genes and 11 are characterized as stress response genes. The group of 9 RNA processing genes comprising: MAGOHB, PAPOLA, PNPT1, POP4, PPIH, RBM39, RPS6, SUPT16H and TXNL4A. The group of 11 stress response genes comprising: EIF2S1, GNA13, GNB1, HLA-DRA, PRDX2, PRDX3, RAD23A, RPS6, RUNX1, SMC6 and SUPT16H. RPS6 and SUPT16H are members of both the stress response group and well as the RNA processing group.

See the description of these genes above as the 55 genes in the present set is the combination of the set of nine biomarkers and the set of 46 biomarkers described above.

36 Biomarkers for Prediction of Disease Free Survival.

In one embodiment, the signature for disease free survival is the group of 36 genes comprising: CAD, CCNK, CDC7, CDT1, CENPH, CHEK1, EZH2, GINS1, HELLS, MCM2, MCM3, MCM4, MCM5, MCM6, MCM7, MYC, POLD1, PRIM1, RFC5, RRM2, SNRPD3, TK1, TYMS, UHRF1, WDHD1, CCDC86, ELOVL6, GABRP, KRT17, MMP12, NUP107, NUTF2, PA2G4, SLC7A5, SQLE, and WASF1. Of this group of 36 genes, 25 genes are related to mitosis. The group of 25 mitotic genes comprising: CAD, CCNK, CDC7, CDT1, CENPH, CHEK1, EZH2, GINS1, HELLS, MCM2, MCM3, MCM4, MCM5, MCM6, MCM7, MYC, POLD1, PRIM1, RFC5, RRM2, SNRPD3, TK1, TYMS, UHRF1 and WDHD1.

CAD (carbamoyl-phosphate synthetase 2, aspartate transcarbamylase, and dihydroorotase, Gene ID: 790) is a gene that encodes a trifunctional protein which is associated with the enzymatic activities of the first 3 enzymes in the 6-step pathway of pyrimidine biosynthesis: carbamoylphosphate synthetase (CPS II), aspartate transcarbamoylase, and dihydroorotase. This protein is regulated by the mitogen-activated protein kinase (MAPK) cascade, which indicates a direct link between activation of the MAPK cascade and de novo biosynthesis of pyrimidine nucleotides.

The expression level of a gene encoding CAD can also be measured by using or detecting the human nucleotide sequence, or a fragment thereof, of GenBank Accession No. NM_{—}004341.3 GI:47458828, Homo sapiens carbamoyl-phosphate synthetase 2, aspartate transcarbamylase, and dihydroorotase (CAD), mRNA, which is expressed as CAD protein [Homo sapiens], GenBank Accession No. NP_{—}004332.2 GI:18105007, the GenBank Accession and Gene information hereby incorporated by reference.

CCNK (cyclin K, Gene ID: 8812) is a gene that encodes a member of the transcription cyclin family. These cyclins may regulate transcription through their association with and activation of cyclin-dependent kinases (CDK) that phosphorylate the C-terminal domain (CTD) of the large subunit of RNA polymerase II. This gene product may play a dual role in regulating CDK and RNA polymerase II activities.

The expression level of a gene encoding CCNK can also be measured by using or detecting the human nucleotide sequence, or a fragment thereof, of GenBank Accession No. NM_{—}001099402.1 GI:150417988, Homo sapiens cyclin K (CCNK), mRNA, which is expressed as cyclin-K [Homo sapiens], GenBank Accession No. NP_{—}001092872.1 GI:150417989, the GenBank Accession and Gene information hereby incorporated by reference.

CDC7 (cell division cycle 7 homolog; Gene ID: 8317) is a gene that encodes a cell division cycle protein with kinase activity that is critical for the G1/S transition. The yeast homolog is also essential for initiation of DNA replication as cell division occurs. Overexpression of this gene product may be associated with neoplastic transformation for some tumors.

The expression level of a gene encoding CDC7 can also be measured by using or detecting the human nucleotide sequence, or a fragment thereof, of GenBank Accession No. NM_{—}001134419.1 GI:197313664, Homo sapiens cell division cycle 7 homolog (S. cerevisiae) (CDC7), transcript variant 2, mRNA, which is expressed as cell division cycle 7-related protein kinase [Homo sapiens], GenBank Accession No. NP_{—}001127891.1 GI:197313665, GenBank Accession No. NM_{—}001134420.1 GI:197313666, Homo sapiens cell division cycle 7 homolog (S. cerevisiae) (CDC7), transcript variant 3, mRNA, which is expressed as cell division cycle 7-related protein kinase [Homo sapiens], GenBank Accession No. NP_{—}001127892.1 GI:197313667 or GenBank Accession No. NM_{—}003503.3 GI:197313663, Homo sapiens cell division cycle 7 homolog (S. cerevisiae) (CDC7), transcript variant 1, mRNA, which is expressed as cell division cycle 7-related protein kinase [Homo sapiens], GenBank Accession No. NP_{—}003494.1 GI:4502715, the GenBank Accession and Gene information hereby incorporated by reference.

CDT1 (chromatin licensing and DNA replication factor 1, Gene ID: 81620) is a gene that encodes a protein involved in the formation of the pre-replication complex that is necessary for DNA replication. The encoded protein can bind geminin, which prevents replication and may function to prevent this protein from initiating replication at inappropriate origins. Phosphorylation of this protein by cyclin A-dependent kinases results in degradation of the protein.

The expression level of a gene encoding CDT1 can also be measured by using or detecting the human nucleotide sequence, or a fragment thereof, of GenBank Accession No. NM_{—}030928.3 GI:188497688, Homo sapiens chromatin licensing and DNA replication factor 1 (CDT1), mRNA, which is expressed as DNA replication factor Cdt1 [Homo sapiens], GenBank Accession No. NP_{—}112190.2 GI:188497689, the GenBank Accession and Gene information hereby incorporated by reference.

CENPH (centromere protein H, Gene ID: 64946) is a gene that encodes a centromere and kinetochore protein that plays a role in centromere structure, kinetochore formation, and sister chromatid separation. The protein colocalizes with inner kinetochore plate proteins CENP-A and CENP-C in both interphase and metaphase. It localizes outside of centromeric heterochromatin, where CENP-B is localized, and inside the kinetochore corona, where CENP-E is localized during prometaphase. It is thought that this protein can bind to itself, as well as to CENP-A, CENP-B or CENP-C. Multimers of the protein localize constitutively to the inner kinetochore plate and play an important role in the organization and function of the active centromere-kinetochore complex.

The expression level of a gene encoding CENPH can also be measured by using or detecting the human nucleotide sequence, or a fragment thereof, of GenBank Accession No. NM_{—}022909.3 GI:21264590, Homo sapiens centromere protein H (CENPH), mRNA, which is expressed as centromere protein H [Homo sapiens], GenBank Accession No. NP_{—}075060.1 GI:12597655, the GenBank Accession and Gene information hereby incorporated by reference.

CHEK1 (CHK1 checkpoint homolog; gene ID 1111) is a kinase with signal transduction function in cell cycle regulation and checkpoint responses. It is involved in the two major parallel DDR pathways, ATR-Chk1 and ATM-Chk2.

The expression level of a gene encoding CHEK1 can also be measured by using or detecting the human nucleotide sequence, or a fragment thereof, of GenBank Accession No. NM_{—}001114122.2 GI:349501056, Homo sapiens Checkpoint Kinase 1 (CHEK1), mRNA, which is expressed as serine/threonine-protein kinase Chk1 isoform 1 [Homo sapiens] NP_{—}001107594.1 GI:166295196, hereby incorporated by reference.

EZH2 (enhancer of zeste homolog 2, Gene ID: 2146) is a gene that encodes a member of the Polycomb-group (PcG) family. PcG family members form multimeric protein complexes, which are involved in maintaining the transcriptional repressive state of genes over successive cell generations. This protein associates with the embryonic ectoderm development protein, the VAV1 oncoprotein, and the X-linked nuclear protein. This protein may play a role in the hematopoietic and central nervous systems.

The expression level of a gene encoding EZH2 can also be measured by using or detecting the human nucleotide sequence, or a fragment thereof, of GenBank Accession No. NM_{—}001203247.1 GI:322506096, Homo sapiens enhancer of zeste homolog 2 (Drosophila) (EZH2), transcript variant 3, mRNA, which is expressed as histone-lysine N-methyltransferase EZH2 isoform c [Homo sapiens], NP_{—}001190176.1 GI:322506097, GenBank Accession No. NM_{—}001203248.1 GI:322506098, Homo sapiens enhancer of zeste homolog 2 (Drosophila) (EZH2), transcript variant 4, mRNA, which is expressed as histone-lysine N-methyltransferase EZH2 isoform d [Homo sapiens], GenBank Accession No. NP_{—}001190177.1 GI:322506099, GenBank Accession No. NM_{—}001203249.1 GI:322506100, Homo sapiens enhancer of zeste homolog 2 (Drosophila) (EZH2), transcript variant 5, mRNA, which is expressed as histone-lysine N-methyltransferase EZH2 isoform e [Homo sapiens], GenBank Accession No. NP_{—}001190178.1 GI:322506101, GenBank Accession No. NM_{—}004456.4 GI:322506095 Homo sapiens enhancer of zeste homolog 2 (Drosophila) (EZH2), transcript variant 1, mRNA, which is expressed as histone-lysine N-methyltransferase EZH2 isoform a [Homo sapiens], GenBank Accession No. NP_{—}004447.2 GI:21361095 or GenBank Accession No. NM_{—}152998.2 GI:322506094, Homo sapiens enhancer of zeste homolog 2 (Drosophila) (EZH2), transcript variant 2, mRNA, which is expressed as histone-lysine N-methyltransferase EZH2 isoform b [Homo sapiens], GenBank Accession No. NP_{—}694543.1 GI:23510384, the GenBank Accession and Gene information hereby incorporated by reference.

GINS1 (GINS complex subunit 1 (Psf1 homolog), Gene ID: 9837) is a gene that codes for a protein that is a subunit of the yeast heterotetrameric GINS complex which is made up of Sld5 (GINS4; MIM 610611), Psf1, Psf2 (GINS2; MIM 610609), and Psf3 (GINS3; MIM 610610). The formation of the GINS complex is essential for the initiation of DNA replication in yeast and Xenopus egg extracts

The expression level of a gene encoding GINS1 can also be measured by using or detecting the human nucleotide sequence, or a fragment thereof, of GenBank Accession No. NM_{—}021067.3 GI:126116593, Homo sapiens GINS complex subunit 1 (Psf1 homolog) (GINS1), mRNA, which is expressed as DNA replication complex GINS protein PSF1 [Homo sapiens], GenBank Accession No. NP_{—}066545.3 GI:126116594, the GenBank Accession and Gene information hereby incorporated by reference.

HELLS (helicase, lymphoid-specific. Gene ID: 3070) is a gene that encodes a lymphoid-specific helicase. Other helicases function in processes involving DNA strand separation, including replication, repair, recombination, and transcription. This protein is thought to be involved with cellular proliferation and may play a role in leukemogenesis.

The expression level of a gene encoding HELLS can also be measured by using or detecting the human nucleotide sequence, or a fragment thereof, of GenBank Accession No. NM_{—}018063.3 GI:27894386, Homo sapiens helicase, lymphoid-specific (HELLS), mRNA, which is expressed as lymphoid-specific helicase [Homo sapiens], GenBank Accession No. NP_{—}060533.2 GI:21914927, the GenBank Accession and Gene information hereby incorporated by reference.

MCM2, MCM3, MCM4, MCM5, MCM6 and MCM7 are genes that encode proteins that are highly conserved mini-chromosome maintenance proteins (MCM) that are involved in the initiation of eukaryotic genome replication. The hexameric protein complex formed by MCM proteins (MCM2-7) is a key component of the pre-replication complex (pre_RC) and may be involved in the formation of replication forks and in the recruitment of other DNA replication related proteins. This MCM2 protein forms a complex with MCM4, 6, and 7, and has been shown to regulate the helicase activity of the complex, and may act as a DNA unwinding enzyme. The phosphorylation of the complex by CDC2 kinase reduces the helicase activity, suggesting a role in the regulation of DNA replication. MCM2 is phosphorylated, and thus regulated by, protein kinases CDC2 and CDC7. MCM3 has been shown to interact directly with MCM5/CDC46 and interacts with and is acetylated by MCM3AP, a chromatin-associated acetyltransferase. The acetylation of MCM3 inhibits the initiation of DNA replication and cell cycle progression. The phosphorylation of this MCM4 protein by CDC2 kinase reduces the DNA helicase activity and chromatin binding of the MCM complex. MCM4 is mapped to a region on the chromosome 8 head-to-head next to the PRKDC/DNA-PK, a DNA-activated protein kinase involved in the repair of DNA double-strand breaks. The MCM5 protein is structurally very similar to the CDC46 protein from S. cerevisiae, a protein involved in the initiation of DNA replication. The MCM5 protein can interact with at least two other members of this family. MCM5 protein is upregulated in the transition from the G0 to G1/S phase of the cell cycle and may actively participate in cell cycle regulation. Single nucleotide polymorphisms in the intron regions of MCM6 gene are associated with differential transcriptional activation of the promoter of the neighboring lactase gene and, thereby, influence lactose intolerance in early adulthood. Cyclin D1-dependent kinase, CDK4, is found to associate with MCM7 protein, and may regulate the binding of this protein with the tumorsuppressor protein RB1/RB.

The expression level of a gene encoding MCM2 (minichromosome maintenance complex component 2, Gene ID: 4171) can also be measured by using or detecting the human nucleotide sequence, or a fragment thereof, of GenBank Accession No. NM_{—}004526.2 GI:33356546,

Homo sapiens minichromosome maintenance complex component 2 (MCM2), mRNA, which is expressed as DNA replication licensing factor MCM2 [Homo sapiens], GenBank Accession No. NP_{—}004517.2 GI:33356547, the GenBank Accession and Gene information hereby incorporated by reference.

The expression level of a gene encoding MCM3 (minichromosome maintenance complex component 3, Gene ID: 4172), can also be measured by using or detecting the human nucleotide sequence, or a fragment thereof, of GenBank Accession No. NM_{—}001270472.1 GI:394582098, Homo sapiens minichromosome maintenance complex component 3 (MCM3), transcript variant 2, mRNA, which is expressed as DNA replication licensing factor MCM3 isoform 2 [Homo sapiens], GenBank Accession No. NP_{—}001257401.1 GI:394582099 or GenBank Accession No. NM_{—}002388.4 GI:394582092, Homo sapiens minichromosome maintenance complex component 3 (MCM3), transcript variant 1, mRNA, which is expressed as DNA replication licensing factor MCM3 isoform 1 [Homo sapiens], GenBank Accession No. NP_{—}002379.3 GI:394582093, the GenBank Accession and Gene information hereby incorporated by reference.

The expression level of a gene encoding MCM4 (minichromosome maintenance complex component 4, Gene ID: 4173), can also be measured by using or detecting the human nucleotide sequence, or a fragment thereof, of GenBank Accession No. NM_{—}005914.3 GI:325651967, Homo sapiens minichromosome maintenance complex component 4 (MCM4), transcript variant 1, mRNA, which is expressed as DNA replication licensing factor MCM4 [Homo sapiens], GenBank Accession No. NP_{—}005905.2 GI:33469919 or GenBank Accession No. NM_{—}182746.2 GI:325651968, Homo sapiens minichromosome maintenance complex component 4 (MCM4), transcript variant 2, mRNA, which is expressed as DNA replication licensing factor MCM4 [Homo sapiens], GenBank Accession No. NP_{—}877423.1 GI:33469917, the GenBank Accession and Gene information hereby incorporated by reference.

The expression level of a gene encoding MCM5 (minichromosome maintenance complex component 5, can also be measured by using or detecting the human nucleotide sequence, or a fragment thereof, of GenBank Accession No. NM_{—}006739.3 GI:143770796, Homo sapiens minichromosome maintenance complex component 5 (MCM5), mRNA, which is expressed as DNA replication licensing factor MCM5 [Homo sapiens], GenBank Accession No. NP_{—}006730.2 GI:23510448, the GenBank Accession and Gene information hereby incorporated by reference.

The expression level of a gene encoding MCM6 (minichromosome maintenance complex component 6, Gene ID: 4175), can also be measured by using or detecting the human nucleotide sequence, or a fragment thereof, of GenBank Accession No. NM_{—}005915.5 GI:386869284, Homo sapiens minichromosome maintenance complex component 6 (MCM6), mRNA, which is expressed as DNA replication licensing factor MCM6 [Homo sapiens], GenBank Accession No. NP_{—}005906.2 GI:7427519, the GenBank Accession and Gene information hereby incorporated by reference.

The expression level of a gene encoding MCM7 (minichromosome maintenance complex component 7, Gene ID: 4176), can also be measured by using or detecting the human nucleotide sequence, or a fragment thereof, of GenBank Accession No. NM_{—}005916.3 GI:33469967, Homo sapiens minichromosome maintenance complex component 7 (MCM7), transcript variant 1, mRNA, which is expressed as DNA replication licensing factor MCM7 isoform 1 [Homo sapiens], GenBank Accession No. NP_{—}005907.3 GI:33469968 or GenBank Accession No. NM_{—}182776.1 GI:33469921, Homo sapiens minichromosome maintenance complex component 7 (MCM7), transcript variant 2, mRNA, which is expressed as lymphoid-specific helicase [Homo sapiens], GenBank Accession No. NM_{—}182776.1 GI:33469921, the GenBank Accession and Gene information hereby incorporated by reference.

MYC (v-myc myelocytomatosis viral oncogene homolog, Gene ID: 4609) is gene that encodes a protein which is a multifunctional, nuclear phosphoprotein that plays a role in cell cycle progression, apoptosis and cellular transformation. It functions as a transcription factor that regulates transcription of specific target genes. Mutations, overexpression, rearrangement and translocation of this gene have been associated with a variety of hematopoietic tumors, leukemias and lymphomas, including Burkitt lymphoma.

The expression level of a gene encoding MYC can also be measured by using or detecting the human nucleotide sequence, or a fragment thereof, of GenBank Accession No. NM_{—}002467.4 GI:239582723, Homo sapiens v-myc myelocytomatosis viral oncogene homolog (avian)(MYC), mRNA, which is expressed as myc proto-oncogene protein [Homo sapiens], GenBank Accession No. NP_{—}002458.2 GI:71774083, the GenBank Accession and Gene information hereby incorporated by reference.

POLD1 (polymerase (DNA directed), delta 1, catalytic subunit, Gene ID: 5424) is a gene that encodes the 125-kDa catalytic subunit of DNA polymerase delta. DNA polymerase delta possesses both polymerase and 3′ to 5′ exonuclease activity and plays a critical role in DNA replication and repair.

The expression level of a gene encoding POLD1 can also be measured by using or detecting the human nucleotide sequence, or a fragment thereof, of GenBank Accession No. NM_{—}001256849.1 GI:379030587, Homo sapiens polymerase (DNA directed), delta 1, catalytic subunit (POLD1), transcript variant 1, mRNA, which is expressed as DNA polymerase delta catalytic subunit [Homo sapiens], GenBank Accession No. NP_{—}001243778.1 GI:379030588, GenBank Accession No. NM_{—}002691.3 GI:379030589, Homo sapiens polymerase (DNA directed), delta 1, catalytic subunit (POLD1), transcript variant 2, mRNA, which is expressed as DNA polymerase delta catalytic subunit [Homo sapiens], GenBank Accession No. NP_{—}002682.2 GI:156616275 or GenBank Accession No. NR_{—}046402.1 GI:379030590, Homo sapiens polymerase (DNA directed), delta 1, catalytic subunit (POLD1), transcript variant 3, non-coding RNA, the GenBank Accession and Gene information hereby incorporated by reference.

PRIM1 (primase, DNA, polypeptide 1 (49 kDa), Gene ID: 5557) is a gene that encodes a protein which is the small, 49 kDa primase subunit. The replication of DNA in eukaryotic cells is carried out by a complex chromosomal replication apparatus, in which DNA polymerase alpha and primase are two key enzymatic components. Primase, which is a heterodimer of a small subunit and a large subunit, synthesizes small RNA primers for the Okazaki fragments made during discontinuous DNA replication.

The expression level of a gene encoding PRIM1 can also be measured by using or detecting the human nucleotide sequence, or a fragment thereof, of GenBank Accession No. NM_{—}000946.2 GI:41349493, Homo sapiens primase, DNA, polypeptide 1 (49 kDa) (PRIM1), mRNA, which is expressed as DNA primase small subunit [Homo sapiens], GenBank Accession No. NP_{—}000937.1 GI:4506051, the GenBank Accession and Gene information hereby incorporated by reference.

RFC5 (replication factor C (activator 1) 5, 36.5 kDa, Gene ID: 5985) is a gene that encodes the 36 kD subunit of the accessory proteins proliferating cell nuclear antigen (PCNA) and replication factor C (RFC) required for elongation of primed DNA templates by DNA polymerase delta and DNA polymerase. RFC, also named activator 1, is a protein complex consisting of five distinct subunits of 140, 40, 38, 37, and 36 kD. This subunit can interact with the C-terminal region of PCNA. It forms a core complex with the 38 and 40 kDa subunits. The core complex possesses DNA-dependent ATPase activity, which was found to be stimulated by PCNA in an in vitro system.

The expression level of a gene encoding RFC5 can also be measured by using or detecting the human nucleotide sequence, or a fragment thereof, of GenBank Accession No. NM_{—}001130112.2 GI:332164787, Homo sapiens replication factor C (activator 1) 5, 36.5 kDa (RFC5), transcript variant 4, mRNA, which is expressed as replication factor C subunit 5 isoform 3 [Homo sapiens], GenBank Accession No. NP_{—}001123584.1 GI:194306569,

GenBank Accession No. NM_{—}001206801.1 GI:332164785, Homo sapiens replication factor C (activator 1) 5, 36.5 kDa (RFC5), transcript variant 5, mRNA, which is expressed as replication factor C subunit 5 isoform 4 [Homo sapiens], GenBank Accession No. NP_{—}001193730.1 GI:332164786, GenBank Accession No. NM_{—}007370.5 GI:332164784, Homo sapiens replication factor C (activator 1) 5, 36.5 kDa (RFC5), variant 1, mRNA, which is expressed as replication factor C subunit 5 isoform 1 [Homo sapiens], GenBank Accession No. NP_{—}031396.1 GI:6677723 or GenBank Accession No. NM_{—}181578.3 GI:332205901, Homo sapiens replication factor C (activator 1) 5, 36.5 kDa (RFC5), transcript variant 2, mRNA, which is expressed as replication factor C subunit 5 isoform 2 [Homo sapiens], GenBank Accession No. NP_{—}853556.2 GI:194306567, the GenBank Accession and Gene information hereby incorporated by reference.

RRM2 (ribonucleotide reductase M2, Gene ID: 6241) is a gene that encodes one of two non-identical subunits for ribonucleotide reductase. This reductase catalyzes the formation of deoxyribonucleotides from ribonucleotides. Synthesis of the encoded protein (M2) is regulated in a cell-cycle dependent fashion.

The expression level of a gene encoding RRM2 can also be measured by using or detecting the human nucleotide sequence, or a fragment thereof, of GenBank Accession No. NM_{—}001034.3 GI:260064011, Homo sapiens ribonucleotide reductase M2 (RRM2), transcript variant 2, mRNA, which is expressed as ribonucleoside-diphosphate reductase subunit M2 isoform 2 [Homo sapiens], GenBank Accession No. NP_{—}001025.1 GI:4557845 or GenBank Accession No. NM_{—}001165931.1 GI:260064012, Homo sapiens ribonucleotide reductase M2 (RRM2), transcript variant 1, mRNA, which is expressed as ribonucleoside-diphosphate reductase subunit M2 isoform 1 [Homo sapiens], GenBank Accession No. NP_{—}001159403.1 GI:260064013, the GenBank Accession and Gene information hereby incorporated by reference.

SNRPD3 (small nuclear ribonucleoprotein D3 polypeptide 18 kDa, Gene ID: 6634) is a gene that encodes a protein which belongs to the small nuclear ribonucleoprotein core protein family. It is required for pre-mRNA splicing and small nuclear ribonucleoprotein biogenesis.

The expression level of a gene encoding SNRPD3 can also be measured by using or detecting the human nucleotide sequence, or a fragment thereof, of GenBank Accession No. NM_{—}004175.3 GI:34328935, Homo sapiens small nuclear ribonucleoprotein D3 polypeptide 18 kDa (SNRPD3), mRNA, which is expressed as small nuclear ribonucleoprotein Sm D3 [Homo sapiens], GenBank Accession No. NP_{—}004166.1 GI:4759160, the GenBank Accession and Gene information hereby incorporated by reference.

TK1 (thymidine kinase 1, soluble, Gene ID: 7083) is a cell cycle-dependent marker that increases dramatically during the S-phase of the cell cycle. Studies have shown that determination of serological TK1 (STK1) in tumour patients provides information for prognosis of cancer patients and for monitoring the outcome of tumour therapy. Two forms of this protein have been identified in animal cells, one in cytosol and one in mitochondria.

The expression level of a gene encoding TK1 can also be measured by using or detecting the human nucleotide sequence, or a fragment thereof, of GenBank Accession No. NM_{—}003258.4 GI:164698437, Homo sapiens thymidine kinase 1, soluble (TK1), mRNA, which is expressed as thymidine kinase, cytosolic [Homo sapiens], GenBank Accession No. NP_{—}003249.3 GI:164698438, the GenBank Accession and Gene information hereby incorporated by reference.

TYMS (thymidylate synthetase, Gene ID: 7298) catalyzes the methylation of deoxyuridylate to deoxythymidylate using 5,10-methylenetetrahydrofolate (methylene-THF) as a cofactor. This function maintains the dTMP (thymidine-5-prime monophosphate) pool critical for DNA replication and repair. The enzyme has been of interest as a target for cancer chemotherapeutic agents. It is considered to be the primary site of action for 5-fluorouracil, 5-fluoro-2-prime-deoxyuridine, and some folate analogs.

The expression level of a gene encoding TYMS can also be measured by using or detecting the human nucleotide sequence, or a fragment thereof, of GenBank Accession No. NM_{—}001071.2 GI:186972144, Homo sapiens thymidylate synthetase (TYMS), mRNA, which is expressed as thymidylate synthase [Homo sapiens], GenBank Accession No. NP_{—}001062.1 GI:4507751, the GenBank Accession and Gene information hereby incorporated by reference.

UHRF1 (ubiquitin-like with PHD and ring finger domains 1 Gene ID: 29128) is a gene that encodes a member of a subfamily of RING-finger type E3 ubiquitin ligases. The protein binds to specific DNA sequences, and recruits a histone deacetylase to regulate gene expression. Its expression peaks at late G1 phase and continues during G2 and M phases of the cell cycle. It plays a major role in the G1/S transition by regulating topoisomerase IIalpha and retinoblastoma gene expression, and functions in the p53-dependent DNA damage checkpoint.

The expression level of a gene encoding UHRF1 can also be measured by using or detecting the human nucleotide sequence, or a fragment thereof, of GenBank Accession No. NM_{—}001048201.1 GI:115430234, Homo sapiens ubiquitin-like with PHD and ring finger domains 1 (UHRF1), transcript variant 1, mRNA, which is expressed as E3 ubiquitin-protein ligase UHRF1 isoform 1 [Homo sapiens], GenBank Accession No. NP_{—}001041666.1 GI:115430235 or GenBank Accession No. NM_{—}013282.3 GI:115430232, Homo sapiens ubiquitin-like with PHD and ring finger domains 1 (UHRF1), transcript variant 2, mRNA, which is expressed as E3 ubiquitin-protein ligase UHRF1 isoform 2 [Homo sapiens], GenBank Accession No. NP_{—}037414.3 GI:115430233, the GenBank Accession and Gene information hereby incorporated by reference.

WDHD1 (WD repeat and HMG-box DNA binding protein 1, Gene ID: 11169) is a gene that encodes a protein that contains multiple N-terminal WD40 domains and a C-terminal high mobility group (HMG) box. WD40 domains are found in a variety of eukaryotic proteins and may function as adaptor/regulatory modules in signal transduction, pre-mRNA processing and cytoskeleton assembly. HMG boxes are found in many eukaryotic proteins involved in chromatin assembly, transcription and replication.

The expression level of a gene encoding WDHD1 can also be measured by using or detecting the human nucleotide sequence, or a fragment thereof, of GenBank Accession No. NM_{—}001008396.2 GI:300794737, Homo sapiens WD repeat and HMG-box DNA binding protein 1 (WDHD1), transcript variant 2, mRNA, which is expressed as WD repeat and HMG-box DNA-binding protein 1 isoform 2 [Homo sapiens], GenBank Accession No. NP_{—}001008397.1 GI:56550086 or GenBank Accession No. NM_{—}007086.3 GI:300794717, Homo sapiens WD repeat and HMG-box DNA binding protein 1 (WDHD1), transcript variant 1, mRNA, which is expressed as WD repeat and HMG-box DNA-binding protein 1 isoform 1 [Homo sapiens], GenBank Accession No. NP_{—}009017.1 GI:5901892, the GenBank Accession and Gene information hereby incorporated by reference.

CCDC86 (coiled-coil domain containing 86, Gene ID: 79080) is a protein coding gene whose function is currently being studied. Studies have shown differential expression of this gene.

The expression level of a gene encoding CCDC86 can also be measured by using or detecting the human nucleotide sequence, or a fragment thereof, of GenBank Accession No. NM_{—}024098.3 GI:153791291, Homo sapiens coiled-coil domain containing 86 (CCDC86), mRNA, which is expressed as coiled-coil domain-containing protein 86 [Homo sapiens], GenBank Accession No. NP_{—}077003.1 GI:13129104, the GenBank Accession and Gene information hereby incorporated by reference.

ELOVL6 (ELOVL fatty acid elongase 6, Gene ID: 79071) is ELOVL family member 6, elongation of very long chain fatty acids (Elov16), which is a microsomal enzyme, which regulates the elongation of C12-16 saturated and monounsaturated fatty acids (FAs). Fatty acid elongases use malonyl-CoA as a 2-carbon donor in the first and rate-limiting step of fatty acid elongation.

The expression level of a gene encoding ELOVL6 can also be measured by using or detecting the human nucleotide sequence, or a fragment thereof, of GenBank Accession No. NM_{—}001130721.1 GI:195539342, Homo sapiens ELOVL fatty acid elongase 6 (ELOVL6), transcript variant 2, mRNA, which is expressed as elongation of very long chain fatty acids protein 6 [Homo sapiens], GenBank Accession No. NP_{—}001124193.1 GI:195539343 or GenBank Accession No. NM_{—}024090.2 GI:195539341, Homo sapiens ELOVL fatty acid elongase 6 (ELOVL6), transcript variant 1, mRNA, which is expressed as elongation of very long chain fatty acids protein 6 [Homo sapiens], GenBank Accession No. NP_{—}076995.1 GI:13129088, the GenBank Accession and Gene information hereby incorporated by reference.

GABRP (gamma-aminobutyric acid (GABA) A receptor, pi, Gene ID: 2568) is a member of the multisubunit chloride channel that mediates the fastest inhibitory synaptic transmission in the central nervous system. The subunit encoded by this gene is expressed in several non-neuronal tissues including the uterus and ovaries. This subunit can assemble with known GABA A receptor subunits, and the presence of this subunit alters the sensitivity of recombinant receptors to modulatory agents such as pregnanolone.

The expression level of a gene encoding GABRP can also be measured by using or detecting the human nucleotide sequence, or a fragment thereof, of GenBank Accession No. NM_{—}014211.2 GI:195976815, Homo sapiens gamma-aminobutyric acid (GABA) A receptor, pi (GABRP), mRNA, which is expressed as gamma-aminobutyric acid receptor subunit pi precursor [Homo sapiens], GenBank Accession No. NP_{—}055026.1 GI:7657106, the GenBank Accession and Gene information hereby incorporated by reference.

KRT17 (keratin 17, Gene ID: 3872) is a gene that condes the type I intermediate filament chain keratin 17, expressed in nail bed, hair follicle, sebaceous glands, and other epidermal appendages. Mutations in this gene lead to Jackson-Lawler type pachyonychia congenita and steatocystoma multiplex.

The expression level of a gene encoding KRT17 can also be measured by using or detecting the human nucleotide sequence, or a fragment thereof, of GenBank Accession No. NM_{—}000422.2 GI:197383031, Homo sapiens keratin 17 (KRT17), mRNA, which is expressed as keratin, type I cytoskeletal 17 [Homo sapiens], GenBank Accession No. NP_{—}000413.1 GI:4557701, the GenBank Accession and Gene information hereby incorporated by reference.

MMP12 (matrix metallopeptidase 12 (macrophage elastase), Gene ID: 4321) is a gene that encodes a protein which is a member of the matrix metalloproteinase (MMP) family which are involved in the breakdown of extracellular matrix in normal physiological processes, such as embryonic development, reproduction, and tissue remodeling, as well as in disease processes, such as arthritis and metastasis. Most MMP's are secreted as inactive proproteins which are activated when cleaved by extracellular proteinases. It is thought that the protein encoded by this gene is cleaved at both ends to yield the active enzyme, but this processing has not been fully described. The enzyme degrades soluble and insoluble elastin. It may play a role in aneurysm formation and studies in mice suggest a role in the development of emphysema. The gene is part of a cluster of MMP genes which localize to chromosome 11q22.3.

The expression level of a gene encoding MMP12 can also be measured by using or detecting the human nucleotide sequence, or a fragment thereof, of GenBank Accession No. NM_{—}002426.4 GI:261878521, Homo sapiens matrix metallopeptidase 12 (macrophage elastase) (MMP12), mRNA, which is expressed as macrophage metalloelastase preproprotein [Homo sapiens], GenBank Accession No. NP_{—}002417.2 GI:73858572, the GenBank Accession and Gene information hereby incorporated by reference.

NUP107 (nucleoporin 107 kDa, Gene ID: 57122) is a gene that encodes a protein who is a member of the nucleoporin family. The protein is localized to the nuclear rim and is an essential component of the nuclear pore complex (NPC). All molecules entering or leaving the nucleus either diffuse through or are actively transported by the NPC.

The expression level of a gene encoding NUP107 can also be measured by using or detecting the human nucleotide sequence, or a fragment thereof, of GenBank Accession No. NM_{—}020401.2 GI:56788371, Homo sapiens nucleoporin 107 kDa (NUP107), mRNA, which is expressed as nuclear pore complex protein Nup107 [Homo sapiens], GenBank Accession No. NP_{—}065134.1 GI:9966881, the GenBank Accession and Gene information hereby incorporated by reference.

NUTF2 (nuclear transport factor 2, Gene ID: 10204) is a gene that encodes a protein which is a cytosolic factor that facilitates protein transport into the nucleus. It interacts with the nuclear pore complex glycoprotein p62. This encoded protein acts at a relative late stage of nuclear protein import, subsequent to the initial docking of nuclear import ligand at the nuclear envelope. It is thought to be part of a multicomponent system of cytosolic factors that assemble at the pore complex during nuclear import.

The expression level of a gene encoding NUTF2 can also be measured by using or detecting the human nucleotide sequence, or a fragment thereof, of GenBank Accession No. NM_{—}005796.1 GI:5031984, Homo sapiens nuclear transport factor 2 (NUTF2), mRNA, which is expressed as nuclear transport factor 2 [Homo sapiens], GenBank Accession No. NP_{—}005787.1 GI:5031985, the GenBank Accession and Gene information hereby incorporated by reference.

PA2G4 (proliferation-associated 2G4, 38 kDa, Gene ID: 5036) is a gene that encodes an RNA-binding protein that is involved in growth regulation. This protein is present in pre-ribosomal ribonucleoprotein complexes and may be involved in ribosome assembly and the regulation of intermediate and late steps of rRNA processing. This protein can interact with the cytoplasmic domain of the ErbB3 receptor and may contribute to transducing growth regulatory signals. This protein is also a transcriptional co-repressor of androgen receptor-regulated genes and other cell cycle regulatory genes through its interactions with histone deacetylases. This protein has been implicated in growth inhibition and the induction of differentiation of human cancer cells. Six pseudogenes, located on chromosomes 3, 6, 9, 18, 20 and X, have been identified.

The expression level of a gene encoding PA2G4 can also be measured by using or detecting the human nucleotide sequence, or a fragment thereof, of GenBank Accession No. NM_{—}006191.2 GI:124494253, Homo sapiens proliferation-associated 2G4, 38 kDa (PA2G4), mRNA, which is expressed as proliferation-associated protein 2G4 [Homo sapiens], GenBank Accession No. NP_{—}006182.2 GI:124494254, the GenBank Accession and Gene information hereby incorporated by reference.

SLC7A5 (solute carrier family 7 (amino acid transporter light chain, L system), member 5, Gene ID: 8140) is involved in sodium-independent, high-affinity transport of large neutral amino acids such as phenylalanine, tyrosine, leucine, arginine and tryptophan, when associated with SLC3A2/4F2hc. It is involved in cellular amino acid uptake and acts as an amino acid exchanger. It is also involved in the transport of L-DOPA across the blood-brain barrier, and that of thyroid hormones triiodothyronine (T3) and thyroxine (T4) across the cell membrane in tissues such as placenta, plays a role in neuronal cell proliferation (neurogenesis) in brain, involved in the uptake of methylmercury (MeHg) when administered as the L-cysteine or D,L-homocysteine complexes, and hence plays a role in metal ion homeostasis and toxicity. It is also involved in the cellular activity of small molecular weight nitrosothiols, via the stereoselective transport of L-nitrosocysteine (L-CNSO) across the transmembrane and may play an important role in high-grade gliomas. It mediates blood-to-retina L-leucine transport across the inner blood-retinal barrier which in turn may play a key role in maintaining large neutral amino acids as well as neurotransmitters in the neural retina and acts as the major transporter of tyrosine in fibroblasts.

The expression level of a gene encoding SLC7A5 can also be measured by using or detecting the human nucleotide sequence, or a fragment thereof, of GenBank Accession No. NM_{—}003486.5 GI:71979931, Homo sapiens solute carrier family 7 (amino acid transporter light chain, L system), member 5 (SLC7A5), mRNA, which is expressed as large neutral amino acids transporter small subunit 1 [Homo sapiens], GenBank Accession No. NP_{—}003477.4 GI:71979932, the GenBank Accession and Gene information hereby incorporated by reference. SQLE (squalene epoxidase, Gene ID: 6713) catalyzes the first oxygenation step in sterol biosynthesis and is thought to be one of the rate-limiting enzymes in this pathway.

The expression level of a gene encoding SQLE can also be measured by using or detecting the human nucleotide sequence, or a fragment thereof, of GenBank Accession No. NM_{—}003129.3 GI:62865634, Homo sapiens squalene epoxidase (SQLE), mRNA, which is expressed as squalene monooxygenase [Homo sapiens], GenBank Accession No. NP_{—}003120.2 GI:62865635, the GenBank Accession and Gene information hereby incorporated by reference.

WASF1 (WAS protein family, member 1, Gene ID: 8936) is a gene that encodes a protein which is a member of the Wiskott-Aldrich syndrome protein (WASP)-family which plays a critical role downstream of Rac, a Rho-family small GTPase, in regulating the actin cytoskeleton required for membrane ruffling. It has been shown to associate with an actin nucleation core Arp2/3 complex while enhancing actin polymerization in vitro. Wiskott-Aldrich syndrome is a disease of the immune system, likely due to defects in regulation of actin cytoskeleton.

The expression level of a gene encoding WASF1 can also be measured by using or detecting the human nucleotide sequence, or a fragment thereof, of GenBank Accession No. NM_{—}001024934.1 GI:68161499, Homo sapiens WAS protein family, member 1 (WASF1), transcript variant 2, mRNA, which is expressed as wiskott-Aldrich syndrome protein family member 1 [Homo sapiens], GenBank Accession No. NP_{—}001020105.1 GI:68161500, GenBank Accession No. NM_{—}001024935.1 GI:68161501, Homo sapiens WAS protein family, member 1 (WASF1), transcript variant 3, mRNA, which is expressed as wiskott-Aldrich syndrome protein family member 1 [Homo sapiens], GenBank Accession No. NP_{—}001020106.1 GI:68161502, GenBank Accession No. NM_{—}001024936.1 GI:6816150, Homo sapiens WAS protein family, member 1 (WASF1), transcript variant 4, mRNA, which is expressed as wiskott-Aldrich syndrome protein family member 1 [Homo sapiens], GenBank Accession No. NP_{—}001020107.1 GI:68161504 or GenBank Accession No. NM_{—}003931.2 GI:68161486, Homo sapiens WAS protein family, member 1 (WASF1), transcript variant 1, mRNA, which is expressed as wiskott-Aldrich syndrome protein family member 1 [Homo sapiens], GenBank Accession No. NP_{—}003922.1 GI:4507913, the GenBank Accession and Gene information hereby incorporated by reference.

The panels of biomarkers described above are also provided in Table 2. In some embodiments, the biomarker panel focused on the baseline signature and shown on the left side of Table 2 is used to provide a prognosis or predicted survival outcome for a patient. In some embodiments, the biomarkers providing the baseline signature comprising the following genes: MAGOHB, PAPOLA, PNPT1, POP4, PPIH, RBM39, SUPT16H, TXNL4A, EIF2S1, GNA13, GNB1, HLA-DRA, RAD23A, SMC6, ABCB10, ABCF1, BAT5, C17orf95, C19orf56, C5orf22, CAP1, CHCHD3, CHCHD4, CLDND1, DDX19A, DNAJC10, GADD45GIP1, GBP1, HLA-B, KIF5B, MCART1, MCM6, MTFR1, NRD1, PDK1, PDXDC1, PEBP1, PHF20, PI4K2B, PIGO, PPME1, SAPS3, SCAND1, SHC1, SNX6 and TM2D2.

This panel may in some embodiments be used as a baseline signature for a patient that has not yet been diagnosed with a cancer.

In one embodiment, the baseline signature panel may also be reduced to a smaller biomarker panel comprising the detection of the genes or gene products of BMP2K, CBX7, CLASP2, PRDX2, PRDX3, RAB6B, RPS6, RUNX1 and SLC15A2. In another embodiment, a method is described for identifying a cancer patient with higher predicted probability of disease free survival, comprising: (a) measuring the amplification or expression level of each gene in the nine biomarker panel in a sample from a patient; and (b) determining a total amplification or expression level of said panel by adding together the measurements from Step (a); and (c) comparing said total in Step (b) to a median of total amplification or expression level of said panel of genes in a normal tissue sample or a reference amplification or expression level, whereby a below-median expression level indicates a patient that has a higher predicted probability of disease free survival.

In other embodiments, the 9-marker baseline panel, representing a subset of Cancer Outcome Associated genes from the 55-marker baseline panel that are significantly associated with disease free survival, would likewise provide broad application since it would provide a method for detection of specific biomarkers that can identify women at increased risk for cancer irrespective of low-dose radiation exposure.

The 9-gene marker panel is shown here below in Table 9.

TABLE 9

| | | |
|---|---|---|
| BMP2K | PRDX2 | RPS6 |
| CBX7 | PRDX3 | RUNX1 |
| CLASP2 | RAB6B | SLC15A2 |

In another embodiment, the 55-marker baseline panel (Table 2a) provides for broad application since it would provide a method for detection of specific biomarkers that can identify women at increased risk for cancer irrespective of low-dose radiation exposure.

In other embodiments, the 46-marker baseline panel, representing the remaining subset of Cancer Outcome Associated genes from the 55-marker baseline panel (minus the 9-marker panel described above), can be used independently to identify women at increased risk for cancer irrespective of low-dose radiation exposure.

In other embodiments, and one specifically focused on the 1 month low-dose response gene set (Table 2b), the 36-marker panel provides a method for detection of specific biomarkers that can identify women at increased risk for cancer after exposure to low dose radiation.

In other embodiments, the 90-marker panel, consisting of all the genes in the above 4 panels minus duplicates, can be used together to provide a method for detection of specific biomarkers that can identify women at increased risk for cancer.

Furthermore, in another embodiment, the present panels described herein may also be used in determining susceptibility to low dose ionizing radiation (LD)-induced cancer in a patient. Several predictor panels of genes that are predictive for increased or decreased susceptibility for LD-induced cancer and predictor panel that are predictive for increased or decreased disease free survival in women who are newly diagnosed with breast cancer.

In one embodiment, an below-median expression of the group of genes that consists of BMP2K, CBX7, CLASP2, PRDX2, PRDX3, RAB6B, RPS6, RUNX1 and SLC15A2 and an above-median expression of the group of genes that consists of MAGOHB, PAPOLA, PNPT1, POP4, PPIH, RBM39, SUPT16H, TXNL4A, EIF2S1, GNA13, GNB1, HLA-DRA, RAD23A, SMC6, ABCB10, ABCF1, BAT5, C17orf95, C19orf56, C5orf22, CAP1, CHCHD3, CHCHD4, CLDND1, DDX19A, DNAJC10, GADD45GIP1, GBP1, HLA-B, KIF5B, MCART1, MCM6, MTFR1, NRD1, PDK1, PDXDC1, PEBP1, PHF20, PI4K2B, PIGO, PPME1, SAPS3, SCAND1, SHC1, SNX6 and TM2D2 indicates a patient is resistant to LD-induced cancers.

Proliferation-Independent Expression Signatures Associated with Disease-Free Survival in Breast Cancer Patients

We have conducted an investigation of gene expression profiling of breast cancer tissue in women who have been newly diagnosed with breast cancer and for whom we have medical and survival information of ˜10 years after diagnosis. Our analyses identified both risky and protective genes that are statistically associated with the duration of disease free survival (DFS) in women diagnosed with breast cancer. We then subdivided the risky and protective genes further into those who genes that were strongly associated with tumor cell proliferation and those that were not. This provided four categories of genes as listed below. Each category of genes includes a full list of genes and a statistically shortened list (signature) that can be used to develop a single value estimate for each category of risk for each woman. The single value for each woman is computed by adding the expression values for the component genes within her tumor tissue. Her level of risk or protection is then estimated by comparing the magnitude of this value against a large reference populations of women. Thus there are four categories with a full list and signature list for each as shown below in Table 10.

TABLE 10

| Category 1 | Category 2 | | Category 4 |
|---|---|---|---|
| RISK-AFF | RISK-UNAFF | Category 3 | PROT-UNAFF |
| GENE | GENE | PROT-AFF | ′GENE |
| RACGAP1 | ABCC5 | SIK3 | CD1C |
| RFK | COX7A2 | PTGDS | SPTAN1 |
| MTCH1 | KIAA0895 | FAM21B | IGLL1/IGLL5 |
| SCAND1 | P4HA2 | KIAA0040 | AHCYL1 |
| PRC1 | GPR56 | ARID5B | GLB1L |
| HRASLS | RECQL4 | ID1 | CD302 |
| ZWINT | PCDH17 | PELI2 | EPHA1 |
| EPRS | DHX15 | CX3CR1 | |
| | HSBP1 | SGK3 | |
| | PPP3CA | | |
| | DHFR | | |

### Example 1

### Determining a 55 Biomarker Predictor Panel

The Baseline Frequencies of Micronuclei in Red Blood Cells and Transcription of 131 Genes in Nucleated White Blood Cells and Mammary Gland Tissues Differ Between BALB/c and C57BL/6 Female Mice.

We used a highly sensitive flow-cytometric assay to assess the frequency of micronucleated red cells as a measure of genome instability in unirradiated young adult female mice (Dertinger S D, Torous D K, Tometsko K R (1997) Flow cytometric analysis of micronucleated reticulocytes in mouse bone marrow. Mutat Res 390: 257-262). FIG. 1B shows that the frequencies of immature reticulocytes (MN-RET) and mature normochromatic erythrocytes (MN-NCE) carrying micronuclei were ˜36% and ˜57% higher in the radiation-sensitive BALB/c strain than in the more radiation-resistant C57BL/6 strain [14] (p<0.0001; Table 4). These differences are at the high end of baseline variation among mouse strains (Bhilwade H N, Chaubey R C, Chauhan P S (2004) Gamma ray induced bone marrow micronucleated erythrocytes in seven strains of mouse. Mutat Res 560: 19-26), and are consistent with the significant associations that have been reported between blood micronuclei frequencies and cancer risks in human studies (Bonassi S, Znaor A, Ceppi M, Lando C, Chang W P, et al. (2007) An increased micronucleus frequency in peripheral blood lymphocytes predicts the risk of cancer in humans. Carcinogenesis 28: 625-631).

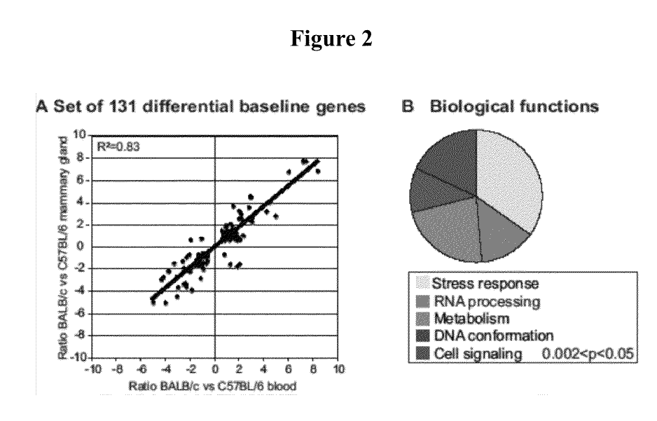

We also compared the transcript profiles for nucleated cells from the peripheral blood and mammary glands (after excising the inguinal lymph nodes) from BALB/c and C57BL/6 female mice to identify common variations in gene expression and their associated tissue functions. FIG. 2A shows that the BALB/c to C57BL/6 transcript ratios for the 131 genes comprising the “systemic baseline signature” are strongly correlated in blood and mammary gland cells (r²=0.83, FIG. 2A). FIG. 2B shows that the systemic baseline signature is significantly enriched for genes involved in stress response and RNA processing and includes DNA repair-associated genes. BALB/c tissues showed lower transcript levels than C57BL/5 for the DNA repair genes PARP3 and RAD23A (not reported previously), and higher transcript levels for MSH5 and SMC6. The PARP3, MSH5, and SMC6 expression findings were confirmed by qPCR (Table 8). LD did not induce or contribute to genomic instability in either strain. We measured the frequencies of micronucleated red blood cells after whole-body LD and HD exposures in sensitive BALB/c and resistant C57BL/6 female mice at the three times indicated in FIG. 1A. The LD exposures induced small transient increases in MN-NCE in both strains at the two early sampling times (p<0.02), but neither strain showed evidence of radiation-induced genomic instability at 1 month after exposure (FIG. 1C). The HD reference exposures (FIGS. 1B and 1C) increased in the frequencies of MN-RET and MN-NCE at the early sampling times in both strains (p<0.0001, Tables 5 and 6). Interestingly, the radiation-sensitive BALB/c showed significant radiation-induced genomic instability at 1 month after HD exposure while the radiation-resistant C57BL/6 mice showed no evidence of this (p<0.0001; FIG. 1B, Tables 5 and 6). We confirmed these finding in a separate study of mice treated with a combined LD/HD regimen where each 7.5 cGy dose was followed 6 hr later by a 1.8 Gy dose (5-6). The BALB/c and C57BL/6 mice that received the combined LD/HD regimen were indistinguishable from the animals that received HD alone, confirming that the LD regimen did not induce or contribute to genomic instability in either strain.

These findings led to the hypothesis that the increased MG cancer risks in BALB/c mice after LD radiation are more likely to be associated with genetically driven differences in oncogenic barriers in their tissues. This motivated our comprehensive comparative analysis of gene expression profiles in the BALB/c and C57BL/6 strains to identify mammary tissue functions that might explain the differences in LD-induced mammary cancer rates.

Early Transcriptional Responses to LD Radiation are Associated with Immune, Epithelial, and Microenvironment Signaling in BALB/c.

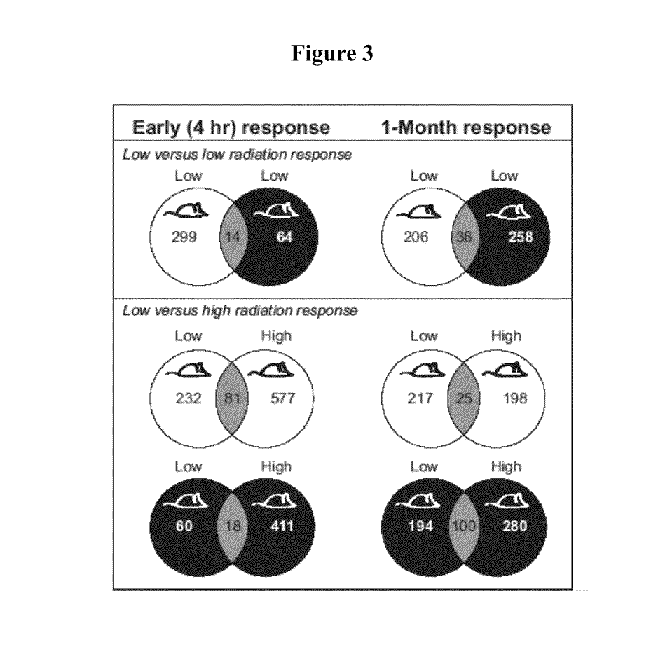

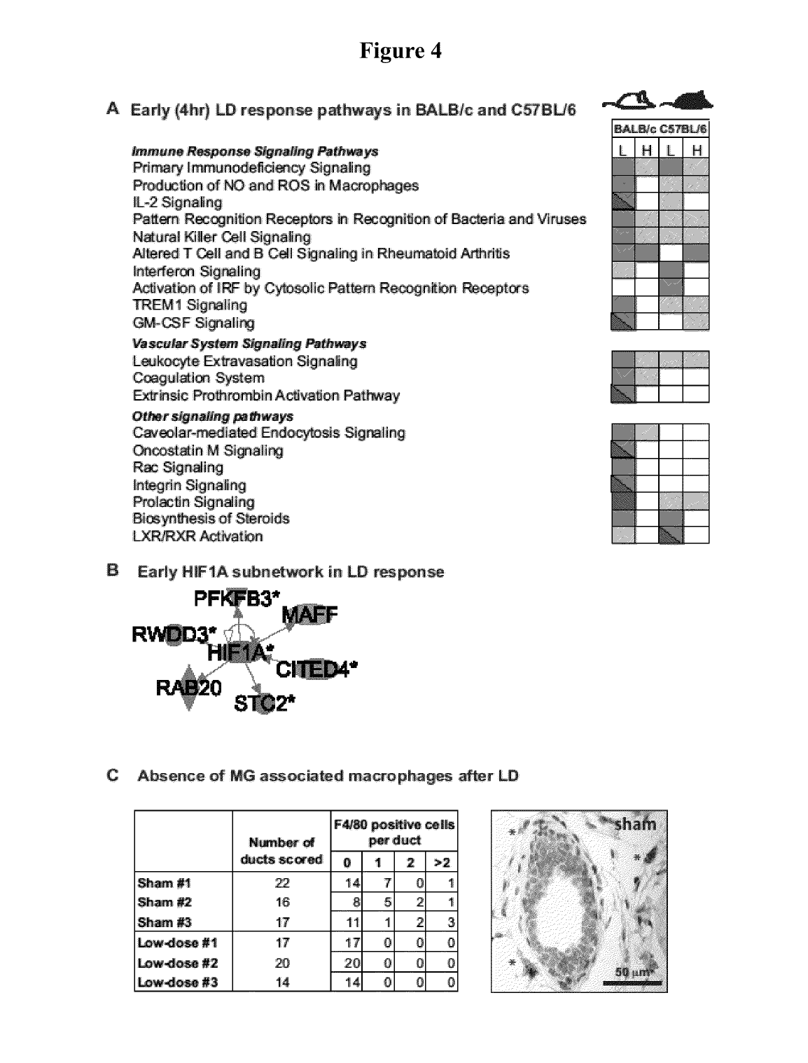

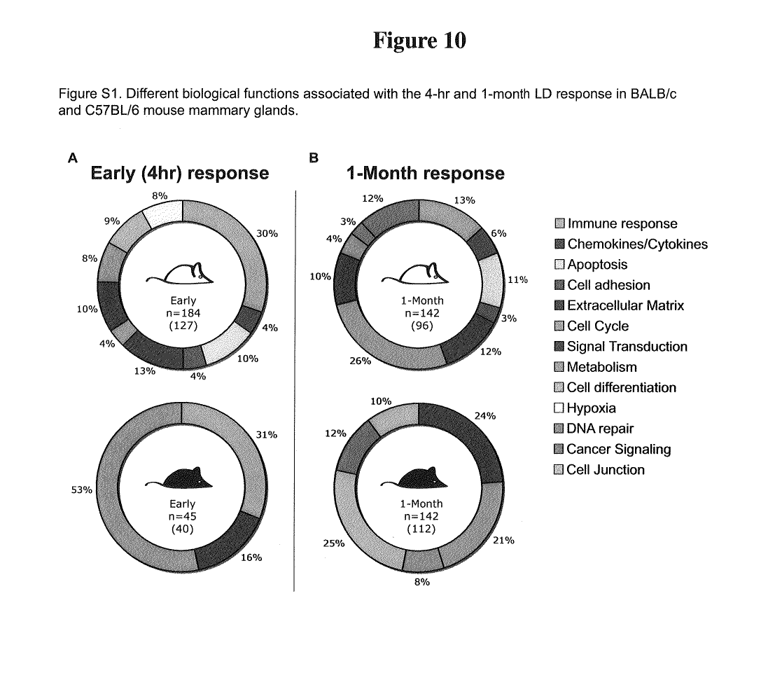

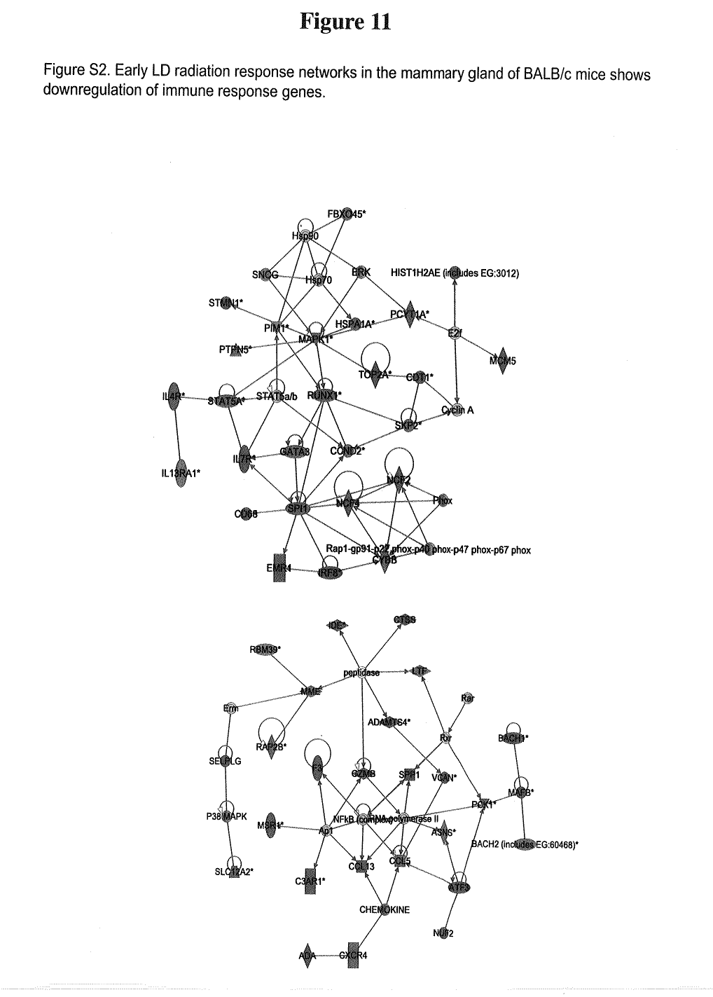

Analysis of transcription profiles in MG tissues from BALB/c and C57BL/6 strains at 4 hrs (i.e., for the early response) and 1 month after LD exposure (FIG. 1A) revealed response functions unique for each strain that were not induced by HD exposures. FIG. 3 shows that there were ˜4× more modulated genes in BALB/c tissue than in C57BL/6 at 4 hours after LD exposure. The differentially expressed gene sets were computationally mapped to curated functions (FIG. 10A), canonical pathways (FIG. 4A), and networks (FIG. 4B, 11). These analyses suggested that early LD responses of BALB/c mice involved down-regulation of immune, epithelial, and microenvironment functions (14 canonical pathways, 0.002<p<0.02), which were not affected by LD in C57BL/6 nor by HD exposure in either strain (FIG. 4A, and Table 7 for an inclusive listing). LD exposure in BALB/c but not C57BL/6 also altered expression in networks consistent with increased HIF1A stress response (FIG. 4B), decreased immune and endothelial function (FIG. 4A), and altered MG developmental- and TGFβ-regulation (Table 1).

The LD induced HIF1A transcription factor network has been associated with cellular responses to genotoxic stress and low 02, (FIG. 4B). L2L analyses (Washington University L2L website) identified 11 genes upregulated in LD-exposed BALB/c mice that were also upregulated under hypoxic conditions in renal epithelial and carcinoma cell systems (4.3e-05<p<9.0e-03) (Harris A L (2002) Hypoxia—a key regulatory factor in tumour growth. Nat Rev Cancer 2: 38-47; Leonard M O, Cottell D C, Godson C, Brady H R, Taylor C T (2003) The role of HIF-1 alpha in transcriptional regulation of the proximal tubular epithelial cell response to hypoxia. J Biol Chem 278: 40296-40304; Jiang Y, Zhang W, Kondo K, Klco J M, St Martin T B, et al. (2003) Gene expression profiling in a renal cell carcinoma cell line: dissecting VHL and hypoxia-dependent pathways. Mol Cancer Res 1: 453-462; Barcellos-Hoff M H, Dix T A (1996) Redox-mediated activation of latent transforming growth factor-beta 1. Mol Endocrinol 10: 1077-1083). The HIF1A, STC2 and RAB20 responses were confirmed by qPCR (Table 8).

The LD induced immune system network in BALB/c (FIG. 4A, 11; Table 7) was associated with lymphocyte activation, and expression of cytokines, chemokines, and macrophage markers. The early BALB/c responses of selected genes in this network (IRF8, IL7R and TREM2) were confirmed by qPCR (Table 8). The early BALB/c specific LD transcriptional response also was associated with down-regulation of coagulation and leukocyte extravasation signaling (FIG. 4A). These functions are an essential part of the inflammatory reaction. Down-regulation of the macrophage-specific marker EMR1 (F4/80) and macrophage-associated proteins (TREM2 and GPNMB) suggested that LD-exposure led to a reduction in macrophages in BALB/c. This prediction was tested and confirmed by IHC in tissue sections of sham and low-dose irradiated BALB/c mice (FIG. 4C; p=0.01).

The early BALB/c specific LD induced transcriptional response signature included ˜90 MG development genes (Table 1). These genes are typically expressed at 3-7 weeks of age during puberty and not at 12 weeks (the age of the irradiated mice). These genes normally are involved in terminal end-bud development (e.g. GATA3, RUNX1, MSX2 and STAT5a), differentiation and ductal branching and morphogenesis. L2L analyses also showed that the set of LD upregulated genes in BALB/c genes were highly associated the developing MG of pubertal mice in other studies (p=5.14e-30) (McBryan J, Howlin J, Kenny P A, Shioda T, Martin F (2007) ERalpha-CITED1 co-regulated genes expressed during pubertal mammary gland development: implications for breast cancer prognosis. Oncogene 26: 6406-6419). CD24, KRT19, WNT4, AREG and IDO1 responses in BALB/c mice were confirmed by qPCR (Table 8). Importantly, ˜50% of these early BALB/c LD response genes (144/313) were TGFβ responsive (Table 1; Website actin.ucd.ie/tgfbeta/, Chen X L, Kapoun A M (2009) Heterogeneous activation of the TGFbeta pathway in glioblastomas identified by gene expression-based classification using TGFbeta-responsive genes. J Transl Med 7: 12). Extracellular TGFβ activation occurs in response to the generation of ROS (Barcellos-Hoff M H, Dix T A (1996) Redox-mediated activation of latent transforming growth factor-beta 1. Mol Endocrinol 10: 1077-1083) and regulates broad epithelial and stromal radiation damage response functions of the BALB/c LD genes. The differential activation of TGFβ responsive □genes that were activated in BALB/c were not activated in C57BL/6 mice, indicating that there is a major genetic difference in TGFβ response to LD radiation in the MGs in these two strains. This is consistent with the increasing evidence of the regulatory role of the TGFβ response in radiation carcinogenesis of the mammary gland (Nguyen D H, Oketch-Rabah H A, Illa-Bochaca I, Geyer F C, Reis-Filho J S, et al. (2011) Radiation Acts on the Microenvironment to Affect Breast Carcinogenesis by Distinct Mechanisms that Decrease Cancer Latency and Affect Tumor Type. Cancer Cell 19: 640-651).

Late MG Transcriptional Responses to LD Radiation are Associated with Proliferation, Senescence, and Microenvironment Function.

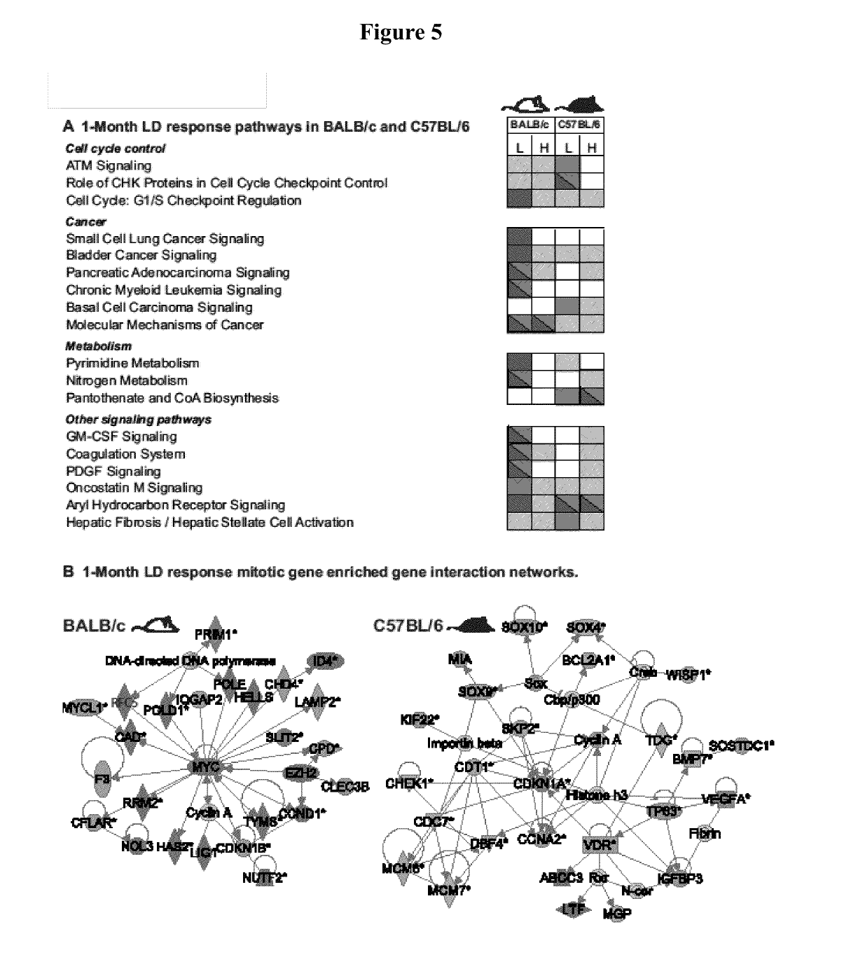

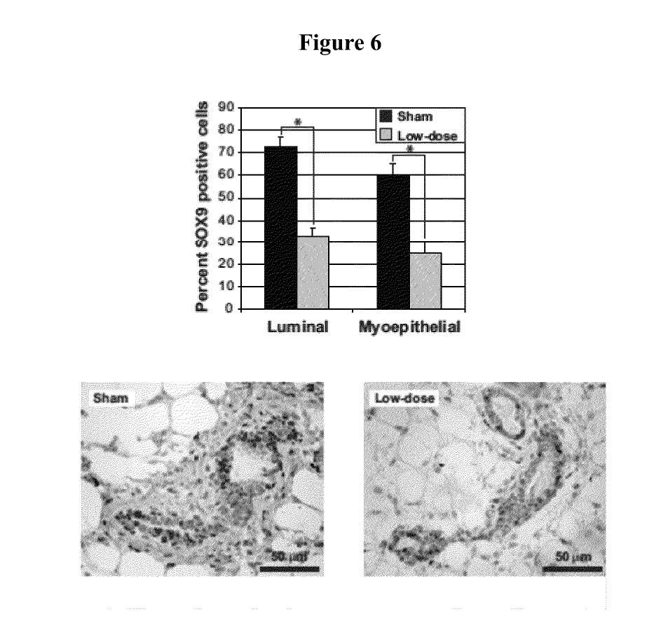

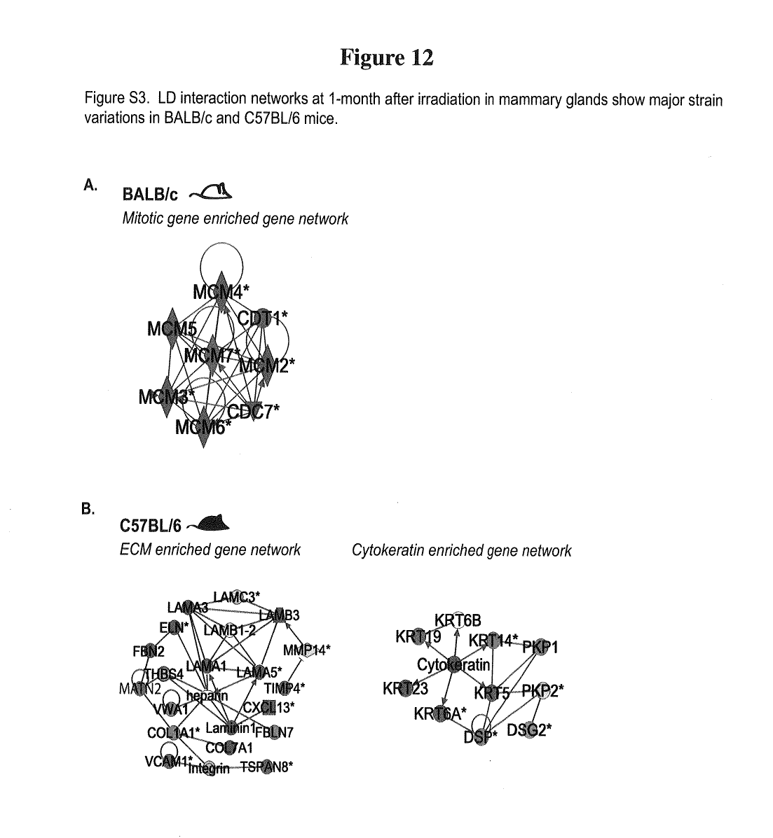

We saw a dramatic transition in the transcript profiles between the early and 1-month responses in MG tissues in radiation-sensitive BALB/c and radiation-resistant C57BL/6 strains of mice. While, similar numbers of genes were modulated in these two strains 1 month after LD exposures (FIG. 3, Table 7), only a few functions significantly modulated at 4 hours in BALB/c remained so 1 month after LD exposure (FIG. 5A, 10). One month after exposure, 5 canonical pathways were uniquely associated with C57BL/6 and a different 11 pathways were unique to BALB/c (FIG. 5A). BALB/c mice acquired an enhanced proliferation phenotype (referenced to sham) while C57BL/6 acquired a diminished proliferation phenotype, consistent with senescence (FIG. 5; 12). The BALB/c 1-month LD response showed upregulation of a MYC-centric network consisting of cell cycle genes (FIG. 5B) plus a subnetwork associated with minichromosome maintenance, (FIG. 12A). In contrast, the 1-month response of C57BL/6 mice showed a highly saturated protein interaction network with the network node, CDKN1A, a negative regulator of cell cycle progression, (FIG. 5B) and with down-regulation of many genes associated with DNA replication, cell-cycle progression and development. L2L analyses of the genes in the C57BL/6-specific senescence signature identified significant associations with expression signatures of cell cycle arrest and senescence (1.13E-11<p<1.15E-04; Chang H Y, Sneddon J B, Alizadeh A A, Sood R, West R B, et al. (2004) Gene expression signature of fibroblast serum response predicts human cancer progression: similarities between tumors and wounds. PLoS Biol 2: E7; Johung K, Goodwin E C, DiMaio D (2007) Human papillomavirus E7 repression in cervical carcinoma cells initiates a transcriptional cascade driven by the retinoblastoma family, resulting in senescence. J Virol 81: 2102-2116; Wu Q, Kirschmeier P, Hockenberry T, Yang T Y, Brassard D L, et al. (2002) Transcriptional regulation during p21WAF1/CIP1-induced apoptosis in human ovarian cancer cells. J Biol Chem 277: 36329-36337) including downregulation of SOX9, SKP2, CCNA2 and CDKN1A (confirmed by qPCR; Table 8 and FIG. 5B). SOX9 is a mark for adult human progenitor cells, mediates deposition of ECM component and is a major transcriptional regulator of mitotic activity in breast cancer (Hanley K P, Oakley F, Sugden S, Wilson D I, Mann D A, et al. (2008) Ectopic SOX9 mediates extracellular matrix deposition characteristic of organ fibrosis. J Biol Chem 283: 14063-14071; Furuyama K, Kawaguchi Y, Akiyama H, Horiguchi M, Kodama S, et al. Continuous cell supply from a Sox9-expressing progenitor zone in adult liver, exocrine pancreas and intestine. Nat Genet 43: 34-41). Consistent with its role in the control of expression of ECM, we observed down-regulation of genes associated with ECM remodeling and epithelial differentiation (not seen in BALB/c mice) suggesting a reduced turn-over of the ECM in C57BL/6 mice (FIG. 12B). FIG. 6 shows that expression of SOX9 protein in the MG was limited to the nuclei of luminal and myoepithelial cells and that the fraction of SOX9-positive cells was significantly reduced after LD exposure in C57BL/6 mice (p<0.0001), consistent with reduced mitotic activity in MG of C57BL/6 mice at 1 month after LD.

LD Regulated Genes in MG are Associated with Human Breast Cancer Survival Duration.

We asked whether any of the strain specific baseline transcripts or LD modulated transcripts were associated with human breast cancer by using human knowledgebases that link expression profiles with breast cancer outcomes.

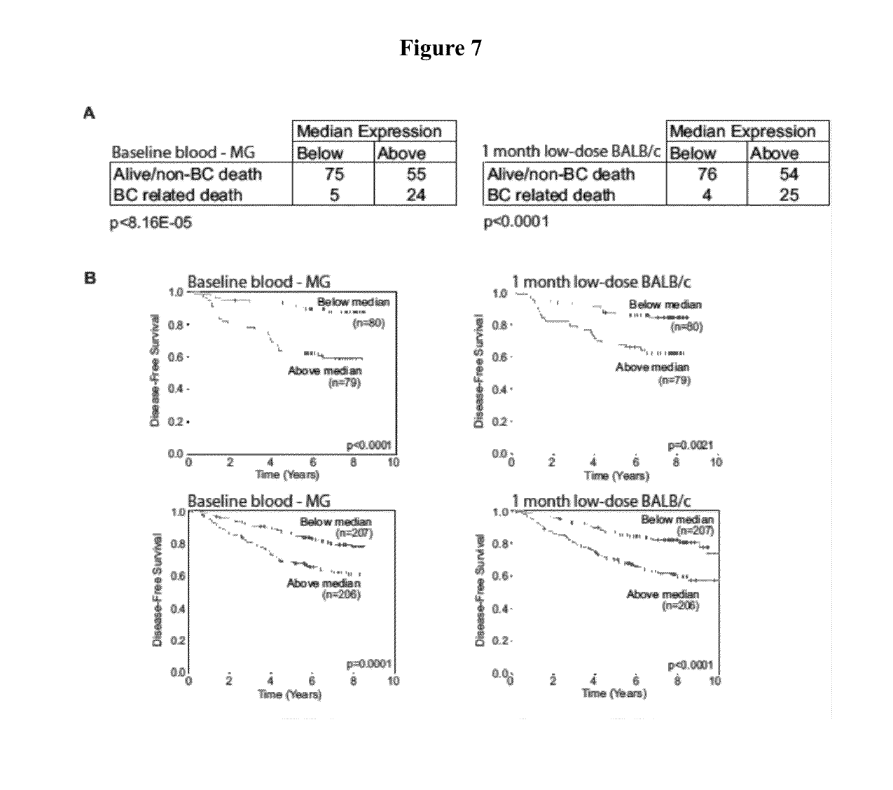

We began by testing the hypothesis that expression levels of the 131 genes that are differentially expressed between non-irradiated BALB/c and C57BL6 mice in blood and MG tissues are associated with breast cancer outcomes. We tested the association of transcript levels for the 94 human orthologs that we were able to associate with the mouse baseline signature with outcome in 156 breast cancer patients for which information on disease-free survival was available (Pawitan Y, Bjohle J, Amler L, Borg A L, Egyhazi S, et al. (2005) Gene expression profiling spares early breast cancer patients from adjuvant therapy: derived and validated in two population-based cohorts. Breast Cancer Res 7: R953-964) We accomplished this by calculating for each patient, the sum of the normalized expression intensities of the human orthologs. As shown in FIG. 7A, patients with above-median expression had significantly reduced survival duration compared to patients with below-median expression (p<8.16 E-05) and had significantly worse prognosis (FIG. 7B; p<0.0001) (Loi S, Haibe-Kains B, Desmedt C, Lallemand F, Tutt A M, et al. (2007) Definition of clinically distinct molecular subtypes in estrogen receptor-positive breast carcinomas through genomic grade. J Clin Oncol 25: 1239-1246; Pawitan Y, Bjohle J, Amler L, Borg A L, Egyhazi S, et al. (2005) Gene expression profiling spares early breast cancer patients from adjuvant therapy: derived and validated in two population-based cohorts. Breast Cancer Res 7: R953-964). Interestingly, murine genes that showed significant strain differences only in MG or only in blood cells did not show significant associations with cancer survival, underscoring the importance of selecting genes that show “systemic differences” across tissues. As a negative control, expression levels of a set of 131 mouse genes that showed the “least” differential expression in both MG and blood between the two strains were not significantly associated with breast cancer survival (p=0.4). Among the 94 human orthologs in the murine baseline signature, we identified 55 cancer outcome associated (COA) genes (Table 2) that individually showed differential expression between the above-median and below-median patient groups (5.3E-12<p<8.4E-03). This set of COA genes was enriched for stress response (11 genes) and RNA processing (9 genes) (FIG. 2B and Table 2). Interestingly, this COA gene set contained a small number of genes (n=9), which showed a significant association with breast cancer survival when expressed at lower levels in the above-median patient group. Among these are a number of previously proposed tumor suppressor genes: RUNX1, CBX7, PRDX2 and PRDX3. Concordant with this human finding, all four genes were also expressed at lower levels in BALB/c compared to C57BL/6 in the systemic baseline signature. This subset of 9 COA genes was significantly associated with disease free survival when down-regulated in the cancer patients (p=5.5E-04). The signature of the remaining 46 genes was associated with disease-free survival when expressed at higher levels (55 minus 9=46 genes) showed a similar association with disease-free survival as compared with the full systemic baseline signature of 94 genes (p=6.8 E-03 vs 8.16E-05).

### Example 2

### Determining a 36 Biomarker Predictor Panel

We then tested the hypothesis that the 1-month BALB/c signature (i.e., the genes that are significantly upregulated at 1 month after LD exposure in relation to sham) was associated with disease-free survival among breast cancer patients. We selected the full and unbiased set of 105 BALB/c genes with significantly increased expression at 1 month after LD exposure. We examined the association of this signature with disease-free survival in breast cancer patients using two human knowledgebases that contain tumor expression profiles obtained at surgery linked to patient survival (Loi S, et al. (2007) J Clin Oncol 25: 1239-124; Pawitan Y, et al. (2005) Breast Cancer Res 7: R953-964). Similar to our analyses of the baseline signature, we summed the expression intensities of all corresponding human orthologs (n=96) from tumor samples and divided the patients into two groups by the median. The patients with “above median” expression values experienced higher rates of breast-cancer related deaths than “below median” patients, (FIG. 7A, p<0.0001) and had significantly worse prognosis (FIG. 7B; p=<0.0021). As a negative control, we selected 105 of the 1-month BALB/c genes with the “least” differential expression between irradiated and sham mice, and showed that the corresponding set of human orthologs was not significantly associated with breast cancer survival (p=0.2). Among the 96 human orthologs of the 1-month BALB/c signature, we identified 36 additional COA genes (Table 2) that individually showed differential expression between the above-median and below-median patient groups (5.4E-14<p<9.2E-03). Of these, 25 were related to mitosis, many of which have individually been associated with breast cancer survival in prior studies. Six of the 11 non-mitosis related genes were previously associated with poor survival in breast cancer patients: KRT17, MMP12, SLC7A5, SQLE, GABRP and PA2G4 (See Furuya M, Horiguchi J, Nakajima H, Kanai Y, Oyama T Correlation of L-type amino acid transporter 1 and CD98 expression with triple negative breast cancer prognosis. Cancer Sci.; Helms M W, Kemming D, Pospisil H, Vogt U, Buerger H, et al. (2008) Squalene epoxidase, located on chromosome 8q24.1, is upregulated in 8q+ breast cancer and indicates poor clinical outcome in stage I and II disease. Br J Cancer 99: 774-780; Liu Z B, Wu J, Ping B, Feng L Q, Di G H, et al. (2009) Basal cytokeratin expression in relation to immunohistochemical and clinical characterization in breast cancer patients with triple negative phenotype. Tumori 95: 53-62; McGowan P M, Duffy M J (2008) Matrix metalloproteinase expression and outcome in patients with breast cancer: analysis of a published database. Ann Oncol 19: 1566-1572; Ou K, Kesuma D, Ganesan K, Yu K, Soon S Y, et al. (2006) Quantitative profiling of drug-associated proteomic alterations by combined 2-nitrobenzenesulfenyl chloride (NBS) isotope labeling and 2DE/MS identification. J Proteome Res 5: 2194-2206; Symmans W F, Fiterman D J, Anderson S K, Ayers M, Rouzier R, et al. (2005) A single-gene biomarker identifies breast cancers associated with immature cell type and short duration of prior breastfeeding. Endocr Relat Cancer 12: 1059-1069), but the remaining 5 had not been previously associated with cancer risk: CCDC86, NUP107, NUTF2, WASF1 and ELOVL6. The association between the 11 non-mitosis genes and breast-cancer-related death was comparable to using the full set of 96 human orthologs or the refined set of 36 mitosis genes suggesting strong involvement of both mitosis and non-mitosis radiation responses in defining the poor prognosis for breast cancer patients.

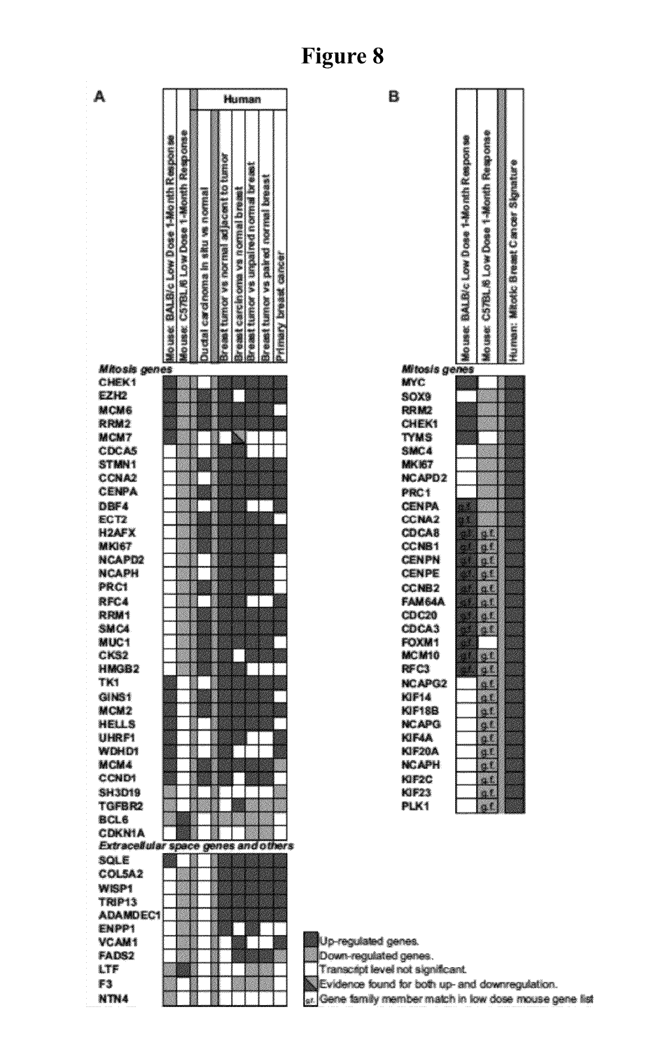

We then addressed the hypothesis that the direction of expression changes in the resistant and sensitive strains at 1-month after LD exposures are concordant with the differences in gene expression of human DCIS and invasive breast cancer, and poor prognosis. (Concordance is defined as up-regulated in BALB/c, no change or down-regulated in C57BL/6 and up in human DCIS or human breast cancer; or vice versa). The BALB/c 1 month COA genes that were associated with poor prognosis when upregulated were enriched for mitosis associated genes (Table 2), whereas in C57BL/6 many of the same genes were down-regulated. As a further test of the importance of concordance in direction of expression, we compared the full set of genes modulated at 1-month after LD exposure in BALB/c and C57BL/6 mice against a meta-gene signature of 946 human breast cancer biomarkers (Abba M C, Lacunza E, Butti M, Aldaz C M (2010) Breast cancer biomarker discovery in the functional genomic age: a systematic review of 42 gene expression signatures. Biomark Insights 5: 103-118), and the direction of expression for overlapping genes was compared against expression in human DCIS and breast cancer (Cheng A S, Culhane A C, Chan M W, Venkataramu C R, Ehrich M, et al. (2008) Epithelial progeny of estrogen-exposed breast progenitor cells display a cancer-like methylome. Cancer Res 68: 1786-1796; Emery L A, Tripathi A, King C, Kavanah M, Mendez J, et al. (2009) Early dysregulation of cell adhesion and extracellular matrix pathways in breast cancer progression. Am J Pathol 175: 1292-1302; Hu Z, Fan C, Livasy C, He X, Oh D S, et al. (2009) A compact VEGF signature associated with distant metastases and poor outcomes. BMC Med 7: 9; Pedraza V, Gomez-Capilla J A, Escaramis G, Gomez C, Tome P, et al. Gene expression signatures in breast cancer distinguish phenotype characteristics, histologic subtypes, and tumor invasiveness. Cancer 116: 486-496; Yu K, Ganesan K, Tan L K, Laban M, Wu J, et al. (2008) A precisely regulated gene expression cassette potently modulates metastasis and survival in multiple solid cancers. PLoS Genet 4: e1000129). This analyses (FIG. 8A) identified 45 concordant genes (34 mitosis and 11 stromal genes) with opposing responses in BALB/c and C57BL/6 where the direction of the BALB/c response matched the direction of response in independent studies of human breast cancers. Furthermore, a subset of 19 genes (FIG. 8A) showed concordant responses between the mouse strains and human DCIS.

Lastly, we tested the association between the 1-month LD BALB/c signature and poor prognosis for human breast cancer (Hu et al, in preparation). FIG. 8B shows strong concordance in the direction of LD-induced expression in mammary tissue in the resistant and sensitive strains of mice and the direction of expression in patients with poor prognosis (FIG. 8B). Hu and colleagues reported that human poor prognosis signature is under transcriptional control of SOX9, which we demonstrated to be down-regulated at both the transcript level and protein level in the resistant C57BL/6 strain (FIGS. 5B and 6).

We employed in vivo systems analyses to identify genetic differences in baseline (i.e., before radiation exposure) and LD-induced mammary gland gene expression in radiation-sensitive and resistant strains of mice that are at the far ends of the mammary cancer sensitivity spectrum (Ullrich R L, Jernigan M C, Satterfield L C, Bowles N D (1987) Radiation carcinogenesis: time-dose relationships. Radiat Res 111: 179-184), and then used in situ protein validation and multi-species bioinformatic resources to identify distinct tissue functions and signatures associated with cancer risks and with properties of breast cancer behavior in humans. Strain variations in baseline and LD-response expression signatures were associated with differential susceptibility to LD-induced mammary cancer in mice and with inter-individual variations in human breast cancer survival. Our findings support the hypothesis that mechanisms that control susceptibility to low-dose radiation induced mammary cancer in mice are similar to those that determine poor-survival in breast cancer patients. In mice, differential baseline expression of tumor suppressor genes and genes associated with stress response and RNA processing, reduced immune activity early after LD exposure and differential expression of proliferation associated genes at 1 month after LD exposure were strongly associated with higher sensitivity to LD-induced mammary tumors in mice. The strong association of baseline and 1-month signatures with disease-free survival in human breast cancer patients points to tissue mechanisms of individual variation to LD-induced mammary cancer, and provide compelling evidence for non-linear dose responses after LD exposures.

Genomic instability is a critical step in the genesis of cancers after high dose exposures (Ullrich R L, Ponnaiya B (1998) Radiation-induced instability and its relation to radiation carcinogenesis. Int J Radiat Biol 74: 747-754; Selvanayagam C S, Davis C M, Cornforth M N, Ullrich R L (1995) Latent expression of p53 mutations and radiation-induced mammary cancer. Cancer Res 55: 3310-3317), and we like others observed that BALB/c was more sensitive to HD-induced genomic instability than C57B1/6 (Ponnaiya B, Cornforth M N, Ullrich R L (1997) Radiation-induced chromosomal instability in BALB/c and C57BL/6 mice: the difference is as clear as black and white. Radiat Res 147: 121-125). Surprisingly, we found no evidence for genomic instability after LD exposures in BALB/c despite its sensitivity to LD-induced mammary cancer, which led us to search for genetic variation in molecular barrier functions that may control susceptibility to LD-induced cancer.

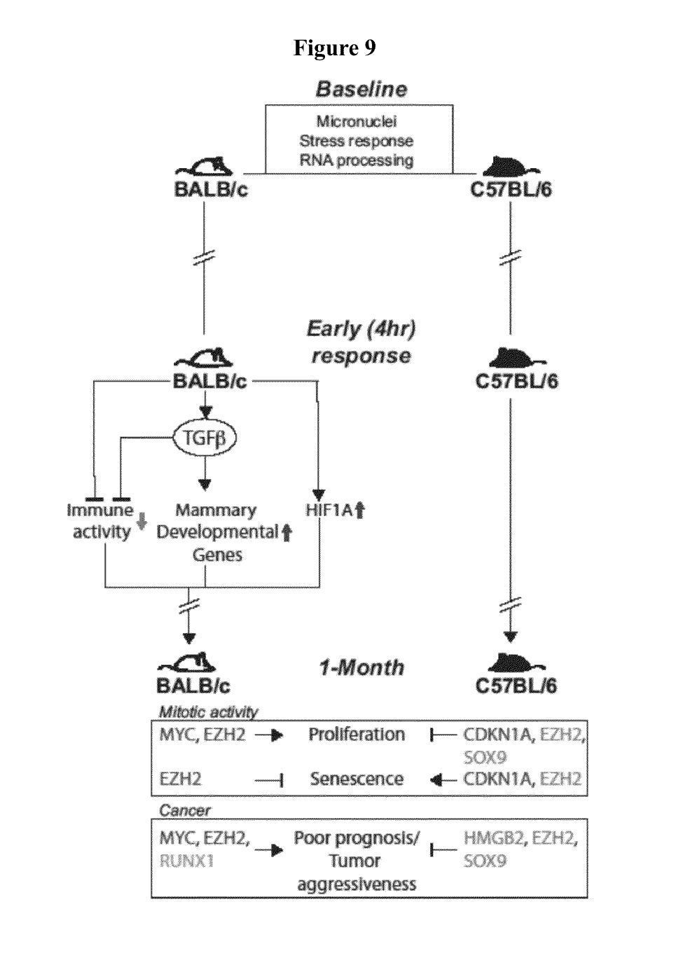

The genetic differences in baseline and LD expression profiles identified several unique tissue response functions associated with mammary cancer risk (FIG. 9). The baseline differences in unirradiated animals (systemic signature) were significantly enriched for stress response and RNA processing genes, and included several DNA repair genes and tumor suppressor genes (Table 2). The stunted immune response observed early after LD-irradiation in BALB/c but not C57BL/6 strains was consistent with prior findings in spleen cells of LD-irradiated BALB/c mice that also received a concanavalin A challenge (Shankar B, Premachandran S, Bharambe S D, Sundaresan P, Sainis K B (1999) Modification of immune response by low dose ionizing radiation: role of apoptosis. Immunol Lett 68: 237-245). As predicted from the microarray data, we demonstrated in tissue sections that the stunted immune response involved reduced numbers of tissue macrophages in BALB/c mice but not C57BL/6 (FIG. 4C), and there are ongoing efforts to examine differential production versus recruitment. The stunted immune response may also be related to the very high TGFβ-associated expression in BALB/c (not seen in C57BL/6 mice), given that 10 cGy exposures can activate TGFβ in BALB/c (Nguyen D H, Oketch-Rabah H A, Illa-Bochaca I, Geyer F C, Reis-Filho J S, et al. (2011) Radiation Acts on the Microenvironment to Affect Breast Carcinogenesis by Distinct Mechanisms that Decrease Cancer Latency and Affect Tumor Type. Cancer Cell 19: 640-651) which in turn can lead to immune dysfunction (Gold L I (1999) The role for transforming growth factor-beta (TGF-beta) in human cancer. Crit Rev Oncog 10: 303-360). The early HIF1A-associated response in BALB/c may indicate tissue hypoxia or a more generalized tissue stress response (Bristow R G, Hill R P (2008) Hypoxia and metabolism. Hypoxia, DNA repair and genetic instability. Nat Rev Cancer 8: 180-192). Among the 12 hypoxia-inducible genes in irradiated BALB/c (Harris A L (2002) Hypoxia—a key regulatory factor in tumour growth. Nat Rev Cancer 2: 38-47; Leonard M O, Cottell D C, Godson C, Brady H R, Taylor C T (2003) The role of HIF-1 alpha in transcriptional regulation of the proximal tubular epithelial cell response to hypoxia. J Biol Chem 278: 40296-40304; Jiang Y, Zhang W, Kondo K, Klco J M, St Martin T B, et al. (2003) Gene expression profiling in a renal cell carcinoma cell line: dissecting VHL and hypoxia-dependent pathways. Mol Cancer Res 1: 453-462), both HIF1α and STC2, a HIF1A target gene, were overexpressed in human cancers and pre-neoplastic breast lesions (Bonin F, Molina M, Malet C, Ginestet C, Berthier-Vergnes 0, et al. (2009) GATA3 is a master regulator of the transcriptional response to low-dose ionizing radiation in human keratinocytes. BMC Genomics 10: 417). The early expression of the transcriptional activators and repressors (i.e., STAT5a, GATA3, RUNX1 and MSX2) may be the reason for the inappropriate expression of the “pubertal-like” mammary development genes in BALB/c mice, which in their TGFβ-modified microenvironment may be associated with the increased cancer risk observed in that strain. In studies with human keratinocytes, the promoter regions of genes modulated by LD radiation of were enriched for GATA3 binding sites, which supports the hypothesis that GATA3 plays an important role in the LD transcriptional response in BALB/c but not C57BL/6 (Bonin F, Molina M, Malet C, Ginestet C, Berthier-Vergnes 0, et al. (2009) GATA3 is a master regulator of the transcriptional response to low-dose ionizing radiation in human keratinocytes. BMC Genomics 10: 417). Finally, our model predicts that at 1-month after LD exposures, the MG tissue of the resistant C57BL/6 mice appears to mount a barrier against cell progression and ECM remodeling, similar to a senescence-like phenotype, which may block the division of residual damaged cells, thereby acting like a global tumor repressor (Rodier F, Campisi J Four faces of cellular senescence. J Cell Biol 192: 547-556). In support of this prediction, we demonstrated that the frequencies of SOX9-protein-positive epithelial cells were reduced in mammary tissue of C57BL/6 (but not in BALB/c) at 1 month after LD exposures. Further research will investigate how these unique baseline and tissue response functions interact at the tissue level to control breast cancer susceptibility.

Comparative systems analyses of the expression profiles in unirradiated mice and human breast cancer outcomes identified 55 genes, each significantly associated with patient survival. In the majority of genes, poor survival was associated with increased expression. Unexpectedly, 9 genes showed the inverse association, including the tumor suppressor genes: RUNX1, CBX7, PRDX2 and PRDX3. These genes were expressed at lower levels both in the blood and MG tissues of unirradiated BALB/c compared to C57BL/6 suggesting that increased cancer sensitivity could be associated with less effective tumor suppressor mechanisms in BALB/c. CBX7 is a known tumor suppressor in both mice and humans and several PRDXs have been shown to have tumor preventive functions (Agrawal-Singh S, Isken F, Agelopoulos K, Klein H U, Thoennissen N H, et al. (2011) Genome wide analysis of histone H3 acetylation patterns in AML identifies PRDX2 as an epigenetically silenced tumor suppressor gene. Blood; Forzati (2012) CBX7 is a tumor suppressor in mice and humans. J Clin Invest; Neumann C A, Fang Q (2007) Are peroxiredoxins tumor suppressors? Curr Opin Pharmacol 7: 375-380). RUNX1 was special in our study, in that it was further down-regulated in BALB/c at 1 month after LD exposure. RUNX1 is a classic tumor suppressor gene in acute myeloid leukemia (AML) and loss of RUNX1 causes hyperproliferation and abnormal morphogenesis in a 3D model of breast epithelial cells (Silva F P, Morolli B, Storlazzi C T, Anelli L, Wessels H, et al. (2003) Identification of RUNX1/AML1 as a classical tumor suppressor gene. Oncogene 22: 538-54; Wang L, Brugge J S, Janes K A Intersection of FOXO- and RUNX1-mediated gene expression programs in single breast epithelial cells during morphogenesis and tumor progression. Proc Natl Acad Sci USA 108: E803-812). The differential baseline expression of tumor suppressor genes, multiple DNA repair and stress response genes in normal blood and mammary tissue of unirradiated BALB/c mice raises the intriguing hypothesis that the collective influence of multiple systemic functions predisposes BALB/c mice mammary carcinogenesis after subsequent LD exposures.

Comparative systems analyses of expression profiles at 1-month after LD exposure in mice and human breast cancer outcomes identified 36 genes that were each associated with patient disease free survival in cancer patients when they were upregulated. This signature includes mitosis-associated genes (Table 2), consistent with the observation that human cancer signatures include proliferation genes and that increased proliferation status of tumors is strongly associated with poor survival (Whitfield M L, George L K, Grant G D, Perou C M (2006) Common markers of proliferation. Nat Rev Cancer 6: 99-106) The statistical association between the 11 non-mitosis genes and breast-cancer-related death was comparable to using the 36 mitosis genes suggesting strong involvement of both mitosis and non-mitosis radiation responses in the 36 gene signature. Five of the 11 non-mitosis genes had not been previously associated with breast cancer survival. We observed minimal overlap between our signatures and the ‘intrinsic’ gene signature that defines the molecular breast cancer subtypes (SQLE and KRT17) and the 70-gene poor prognosis signature (MCM6) (Sorlie T, Perou C M, Tibshirani R, Aas T, Geisler S, et al. (2001) Gene expression patterns of breast carcinomas distinguish tumor subclasses with clinical implications. Proc Natl Acad Sci USA 98: 10869-10874; van 't Veer L J, Dai H, van de Vijver M J, He Y D, Hart A A, et al. (2002) Gene expression profiling predicts clinical outcome of breast cancer. Nature 415: 530-536), suggesting that our LD radiation-response signatures are different from those developed independently of radiation exposures and provide new information related to breast cancer risks from LD radiation exposures.

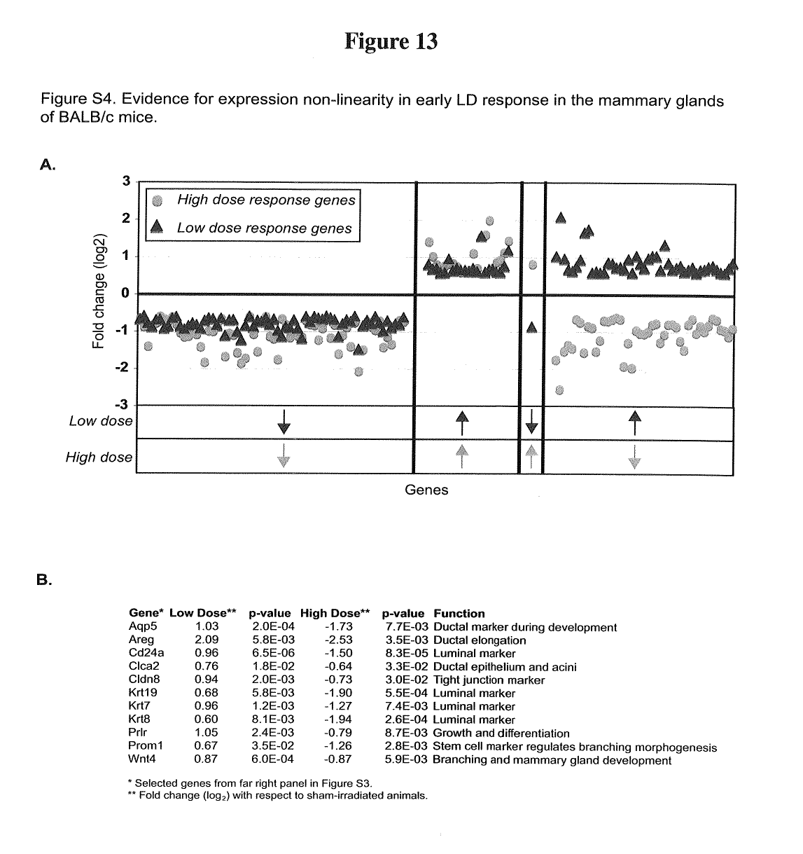

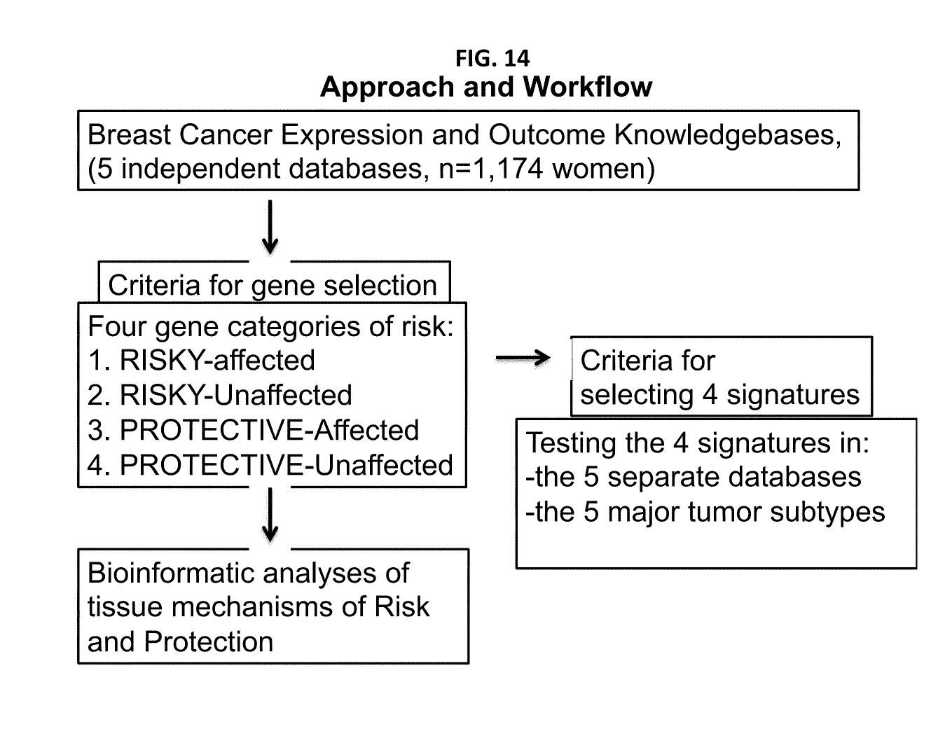

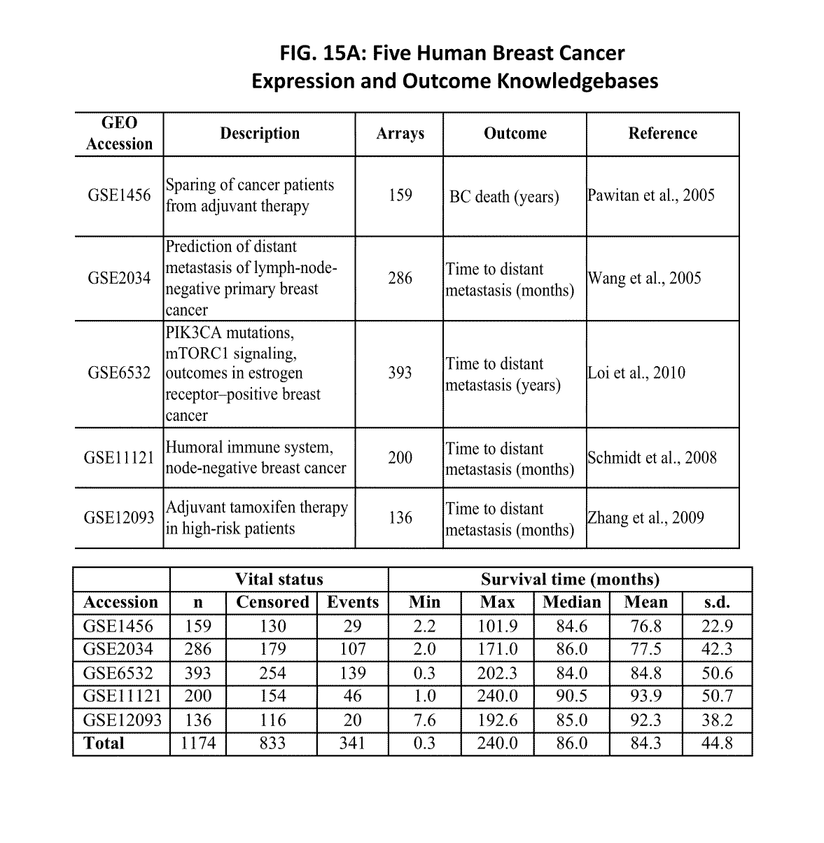

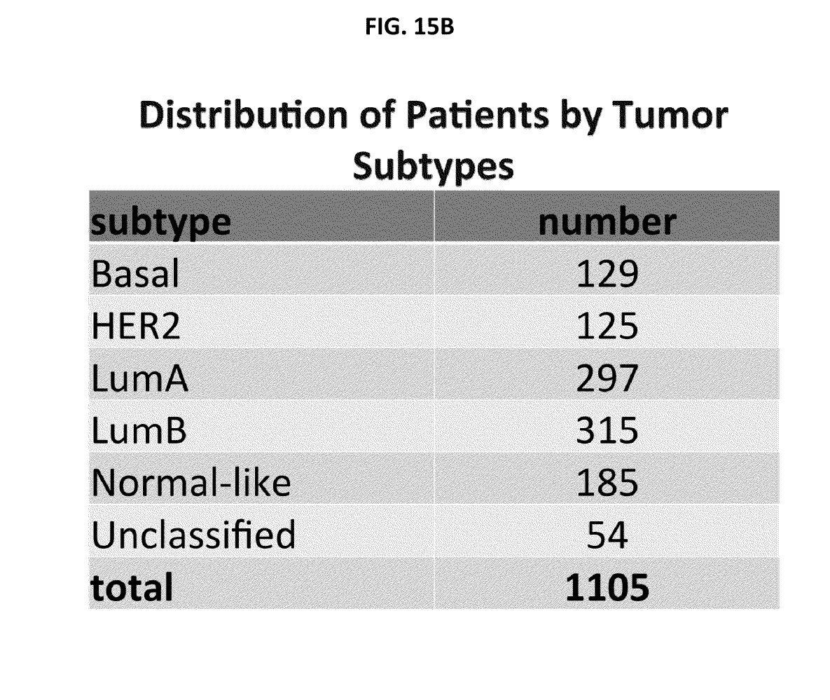

Our studies provide strong evidence for dose non-linearities in expression and tissue functions after LD exposures, and strong evidence against the validity of the LNT for molecular responses. The LNT model uses linear extrapolations of cancer risk from high to LD exposures, with the assumption that underlying mechanisms are also linear. Our study provides overwhelming evidence for dose non-linearities in gene expression (FIG. 3), tissue functions (FIG. 10) and canonical pathways (FIG. 4; Table 7). We also found a number of genes showing plateau-like responses with dose. As shown in FIG. 13, 76 BALB/c genes were modulated in the same direction and at similar magnitudes after low-versus high-dose exposures, in striking contrast to the 24-fold difference in doses. We also found opposing directions for low-versus high-dose responses (FIG. 13B). But most surprising in regards to non-linearity was our finding of strain differences in low-dose thresholds of induction, with the sensitive BALB/c strain showing lower thresholds. Table 3 lists genes similarly modulated at high dose in both BALB/c and C57BL/6 but that differed dramatically in their low-dose responses. While the BALB/c low dose responses were generally lower compared to their high-dose responses, none of these genes were induced after low dose in C57BL/6. Interestingly, the magnitude of the C57BL/6 high-dose responses were significantly different from the BALB/c high-dose responses (p=0.003), but were not different from the BALB/c low-dose responses (p=0.7). Taken together our findings provide strong evidence that the high dose response is not an enhancement of the low-dose response, rather it is remarkably different and strongly argues, at least at the gene expression level, against using the LNT model for low-dose risk predictions.

The fractionated low-dose exposure regimen used in our study is relevant to various human LD radiation exposure scenarios. The maximum yearly allowable dose for radiation workers in the recent nuclear crisis at a Japanese nuclear power plant is 100-250 mSv, which is similar to the whole body fractionated dosing used in our study (4×75=300 mSv). Also, multiple abdominal CT scans can yield doses of ˜60 mGy and full body CT scans can involve doses of ˜100 mGy, similar to the individual doses in our study. Also, the penumbra of radiotherapy fields for breast cancer can deliver doses to the contralateral breast similar to the doses in our study (Boice J D, Jr., Harvey E B, Blettner M, Stovall M, Flannery J T (1992) Cancer in the contralateral breast after radiotherapy for breast cancer. N Engl J Med 326: 781-785).

On the assumption that there is substantial genetic variation in molecular tissue responses and mammary cancer risks in human women exposed to LD ionizing radiation, as observed in mice, our findings provide a novel approach for developing predictive tools to identify individual with higher or lower cancer risks from LD exposures, and for distinguishing breast cancers induced by LD radiation versus other causes. Our work also points to a re-examination of the assumptions associated with biological processes controlling transduction of low-dose radiation into breast cancer risk and suggest a new strategy to identify genetic and molecular features that predispose or protect individuals from LD-induced breast cancer.

### Methods and Materials.

Ethics Statement:

Female, virgin C57BL/6 and BALB/c mice (˜6 weeks old; Harlan Laboratories, Livermore, Calif.) were acclimatized for 2 weeks, and the study was carried out in strict accordance with the Guide for the Care and Use of Laboratory Animals of the National Institutes of Health. The protocol was approved by the Committee on the Ethics of Animal Experiments of the Lawrence Berkeley National Laboratory (Approval number: 25001). At 8 weeks of age, mice (n=6 per group) were exposed to 4 weekly doses of (a) 7.5 cGy, (b) 1.8 Gy, or (c) sham, using a Pantak 320 kVp X-ray machine, operated at 300 kV (2 mA and 196 mGy/min for low dose, 10 mA and 783 mGy/min for high dose).

For the analyses of micronucleated red blood cells, peripheral blood was collected from each mouse at 6 days after each weekly irradiation and at 28 days after the fourth irradiation. Approximately 100 μl of blood was collected per time point from the saphenous vein (Hem A, Smith A J, Solberg P (1998) Saphenous vein puncture for blood sampling of the mouse, rat, hamster, gerbil, guinea pig, ferret and mink. Lab Anim 32: 364-368) and processed with the MicroFlow^{BASIC} kit for the mouse (Litron Laboratories, Rochester, N.Y.) according to the manufacturer's instructions. Samples were kept at −80° C. until shipment to Litron Laboratories where they were analyzed by flow cytometry for the frequencies of micronucleated reticulocytes (MN-RET) and micronucleated normochromatic erythrocytes (MN-NCE) (Dertinger S D, Torous D K, Tometsko K R (1997) Flow cytometric analysis of micronucleated reticulocytes in mouse bone marrow. Mutat Res 390: 257-262). Frequencies of MN-RET and MN-NCE of exposed mice were compared against the respective frequencies in sham of that same strain by ANOVA with Dunnett adjustment for multiple comparisons. A p-value less than 0.05 was considered significant. Differences in baseline MN frequencies were compared using two-tailed T-test with unequal variance.

At 4 hours and 1-month after the last exposure we harvested the 4^{th} pair of mammary glands and removed their inguinal lymph nodes; mice were randomized and individually processed for RNA isolations. Microarray hybridizations were carried out using Affymetrix's HT Mouse Genome 430A 96-Array Plate. The data has been deposited at NCBI GEO. RMA was used to create an expression matrix and NUSE was used to assess array quality. The following available or online bioinformatics software tools and databases were used: L2L Website for depts.washington.edu/121/), KEGG (Website bioinfo.vanderbilt.edu/webgestalt/); DAVID (david.abcc.ncifcrf.gov/), pubertal mammary gland development genes (McBryan J, Howlin J, Kenny P A, Shioda T, Martin F (2007) ERalpha-CITED1 co-regulated genes expressed during pubertal mammary gland development: implications for breast cancer prognosis. Oncogene 26: 6406-641), TGFβ-responsive genes (Website for actin.ucd.ie/tgfbeta/ and Chen XL, Kapoun A M (2009) Heterogeneous activation of the TGFbeta pathway in glioblastomas identified by gene expression-based classification using TGFbeta-responsive genes. J Transl Med 7: 12), 942 biomarkers of breast cancer (Abba M C, Lacunza E, Butti M, Aldaz C M (2010) Breast cancer biomarker discovery in the functional genomic age: a systematic review of 42 gene expression signatures. Biomark Insights 5: 103-118) and gene expression in human DCIS and breast cancers (Website for NextBio).

Expression levels of human orthologs of overlapping genes in blood and mammary gland tissue of unirradiated BALB/c and C57BL/6 mice and an unbiased set of 105 up-regulated 1-month BALB/c-specific low dose genes (i.e., genes not up-regulated in C57BL/6) were summed in breast cancer samples of patients from two independent curated breast cancer data sets (GSE1456 and GSE6532) (Loi S, Haibe-Kains B, Desmedt C, Lallemand F, Tutt A M, et al. (2007) Definition of clinically distinct molecular subtypes in estrogen receptor-positive breast carcinomas through genomic grade. J Clin Oncol 25: 1239-124; Pawitan Y, Bjohle J, Amler L, Borg A L, Egyhazi S, et al. (2005) Gene expression profiling spares early breast cancer patients from adjuvant therapy: derived and validated in two population-based cohorts. Breast Cancer Res 7: R953-964). The median expression value was used as a cut-point to assess group survival outcomes. A Kaplan-Meier disease-free survival curve was generated for patients with above median and below median expression. Log-rank tests were performed to compare the difference in disease-free survival between patients in the two clusters.

RNA Isolations and Microarray Hybridization.

Blood was collected from the heart into a heparinized syringe and immediately transferred to RNA later (Ambion). RNA was isolated using the Blood-Ribopure blood kit (Ambion) followed by Globin clear kit following the manufacturer's recommendations. The 4^{th} mammary gland pair was harvested and the inguinal lymph node as well as a second lymph node often present in the distal part of the inguinal mammary gland were removed Mammary tissues were snap frozen in liquid nitrogen within 10 minutes of the euthanasia procedure. Total RNA was isolated by homogenizing the frozen tissue in Trizol reagent (Invitrogen) followed by phase separation using chloroform. RNA was further purified by Qiagen's RNeasy mini kit (74104) and DNA was removed using Qiagen's DNase free kit (79254). RNA samples with RIN numbers greater than 7 were used for further analyses. Microarray hybridizations were performed in the Lawrence Berkeley National Laboratory's HTA facility using Affymetrix's HT Mouse Genome 430A 96-Array Plate. Robust multi-array average (RMA) was then used to create an expression matrix from Affymetrix data. The normalized unscaled standard error (NUSE) plot was generated to visualize the chip-wise distribution of standard error estimates obtained for each gene on each array when performing the robust multichip probe-level fit. Any array with the upper quartile NUSE value greater than 1.10 was removed from the analysis. A statistical hypothesis testing method based on moderated t-statistic is used for detecting differential expression, which is implemented through the R limma package. Gene fold-changes were calculated with respect to sham irradiated animals and gene lists were prepared based on fold-change (log 2 0.58) and p-value (0.1 for low-dose; 0.01 for high-dose). Gene lists were analyzed using Ingenuity Pathway Analysis, the L2L microarray comparison tool (Website of depts.washington.edu/1211), KEGG pathway analysis for Website bioinfo.vanderbilt.edu/webgestalt/) and DAVID GO gene ontology (Website for david.abcc.ncifcrf.gov/; p≦0.05) Dennis, G, Jr., et al., DAVID: Database for Annotation, Visualization, and Integrated Discovery. Genome Biol, 2003. 4(5): p. P3. Select expression array findings were confirmed using quantitative RT PCR analysis following standard methods. β-Actin expression was used as endogenous control. Fold changes were calculated with respect to average of four sham replicates and represented in log 2.

Mouse and Human Database Comparisons.

Low dose BALB/c and C57BL/6 gene lists were compared against a gene expression signature containing pubertal mammary gland development genes (McBryan J, Howlin J, Kenny P A, Shioda T, Martin F (2007) ERalpha-CITED1 co-regulated genes expressed during pubertal mammary gland development: implications for breast cancer prognosis. Oncogene 26: 6406-6419), known TGFβ-responsive genes (See website at actin.ucd.ie/tgfbeta/ and Chen X L, Kapoun A M (2009) Heterogeneous activation of the TGFbeta pathway in glioblastomas identified by gene expression-based classification using TGFbeta-responsive genes. J Transl Med 7: 12), and a meta-analysis of 42 gene expression signatures of breast cancer (Abba M C, Lacunza E, Butti M, Aldaz C M (2010) Breast cancer biomarker discovery in the functional genomic age: a systematic review of 42 gene expression signatures. Biomark Insights 5: 103-11). NextBio (Website for NextBio) was used to retrieve the direction of expression of low-dose genes in DCIS and breast cancer (Cheng A S, Culhane A C, Chan M W, Venkataramu C R, Ehrich M, et al. (2008) Epithelial progeny of estrogen-exposed breast progenitor cells display a cancer-like methylome. Cancer Res 68: 1786-1796; Yu K, Ganesan K, Tan L K, Laban M, Wu J, et al. (2008) A precisely regulated gene expression cassette potently modulates metastasis and survival in multiple solid cancers. PLoS Genet 4: e1000129). Genes were queried in NextBio applying the following filters: “human: as organism, “breast cancer” as key word and “disease versus non-disease” option to find the directionality of expression in transcript profiling studies of cancer versus non-cancer breast tissues

Baseline Strain Difference Signature and Low-Dose Expression Signature at 1-Month after Irradiation as Prognostic Indicators in Breast Cancer Patients.

Human homologs of mouse genes were identified for two separate gene lists: (1) baseline gene expression differences between BALB/c and C57BL/6 in mammary gland and blood (131 genes) and (2) up-regulated late BALB/c genes that were not up-regulated in C57BL/6 (105 genes). The baseline and low-dose gene lists were compared against U133A Affymetrix expression array, which identified 94 and 96 common genes, respectively. Expression levels of these genes in human breast cancer patients of a curated breast cancer data set (GSE1456) were summed and mean-normalized (Pawitan Y, Bjohle J, Amler L, Borg A L, Egyhazi S, et al. (2005) Gene expression profiling spares early breast cancer patients from adjuvant therapy: derived and validated in two population-based cohorts. Breast Cancer Res 7: R953-964). The median expression value was used as a cut-point to assign patients to either “above-median” expression and “below-median” expression and to assess patient outcome, significance was tested using chi-square test. The average of the mean-normalized summed expression values was calculated for each breast cancer subtype. Kaplan-Meier disease-free survival curves were generated comparing the above-median patient group with the below-median patient group in two curated breast cancer data sets (GSE1456 alone and combined with GSE6532) (Loi S, Haibe-Kains B, Desmedt C, Lallemand F, Tutt A M, et al. (2007) Definition of clinically distinct molecular subtypes in estrogen receptor-positive breast carcinomas through genomic grade. J Clin Oncol 25: 1239-1246; Pawitan Y, Bjohle J, Amler L, Borg A L, Egyhazi S, et al. (2005) Gene expression profiling spares early breast cancer patients from adjuvant therapy: derived and validated in two population-based cohorts. Breast Cancer Res 7: R953-964). Log-rank tests were performed to compare the difference in disease-free survival between patients in the two clusters.

Immunohistochemical Analyses.

Following heat-mediated antigen retrieval, sections were processed for immunohistochemistry by blocking with serum (1:10 dilution) corresponding to the species of the biotinylated secondary antibody. Sections were incubated overnight with SOX9 primary antibody at 1:200 dilution (Millipore, AB5535) or F4/80 primary antibody at 1:500 dilution (Abcam, Ab6640). Staining was visualized using the Vectastain ABC kit (Vector Labs) and DAB/H₂O₂. Sections were counterstained with hematoxylin, rinsed in deionized water, differentiated in a 1% acid alcohol solution, rinsed in deionized water and blued in Scott's water. Sections were rinsed in deionized water, dehydrated through graded alcohols and cleared in xylene. Sections were coverlipped using Permount. For each mammary gland, approximately 1000 luminal and myoepithelial cells were counted from 2 mice per group, blinded.

Design Limitations.

Our study was limited in that we used only two strains that differed in their radiation sensitivity, and it remains to be investigated whether similar damage responses will be identified in mammary tissue of other mammary cancer sensitive and resistant strains of mice (e.g., C3H and SPRET/EI). The time-points chosen for our studies (4 hrs and 1 month after the last irradiation) are far removed from the time when low-dose radiation induced mammary cancers manifest themselves, and a more detailed post radiation time response study is warranted. Our transcript profiles used a gross mixture of mammary cell types, which limits our ability to assign functions to specific cell types. To reduce mammary tissue complexity, we removed the inguinal lymph node before transcriptional analyses

### Example 3

### Determining a Four Signature Panel

We are seeking to understand the molecular tissue mechanisms that confer variation in risk or protection for disease-free survival (DSF) in women diagnosed with breast cancer. Using comparative genomics, we utilize human breast cancer knowledgebases that link tumor expression profiling with disease outcome, and expression databases of mammary and blood tissue of inbred strains of mice that differ in their sensitivities for mammary cancer. We have analyzed the expression profiles of diagnostic breast tissue from 1,174 cancer patients representing 5 independent databases. This analyses has identified genes that are significantly associated with decreased (risky) or increased (protective) DFS and that are also either associated with or independent of tumor proliferation status, a known predictor of cancer risk. The influence of proliferation status was assessed by adjustment with a meta-PCNA index consisting of ˜130 known proliferation genes.

We identified four groups of genes in tumor tissue whose expression was significantly associated with DFS across the 5 independent knowledgebases: (1) ˜180 risky genes for DFS were modified to non-significance after adjustment for proliferation status using the meta-PCNA index (RISKY-AFF); (2) ˜50 risky genes were unaffected by the proliferation adjustment (RISKY-UNAFF); (3) ˜100 protective genes were affected to non-significance after proliferation adjustment (PROTECTIVE-AFF); and (4) ˜40 protective genes were unaffected by adjustment (PROTECTIVE-UNAFF). Step-wise Cox regressions identified smaller signatures of ˜10 genes per group that retained the statistical significance of the group. Using bioinformatic tools, the genes of the RISKY-AFF group are strongly associated with cancer signaling and replication functions, as expected. The RISKY-UNAFF group includes genes associated with connective tissue disorders and mitochondrial dysfunction. The PROTECTIVE-AFF group includes tumor suppressors and genes that control cell movement and various breast stromal functions. The PROTECTIVE-UNAFF group involves death receptor, apoptosis and immune functions.

The criteria for selecting risky and protective genes for disease-free survival (DSF) was: (a) Genes of which DSF was significantly associated with RISK in at least 3 databases before PCNA adjustment. Risky-affected: adjustment reduced that number by at least 3. Risky-unaffected: adjustment changed the significance in 0 or 1 database. (b) Genes of which DSF was significantly associated with PROTECTION in at last 3 databases before PCNA adjustment. Protective-affected: adjustment reduced that number by at least 3. Protective-unaffected: adjustment changed the significance in 0 or 1 database.

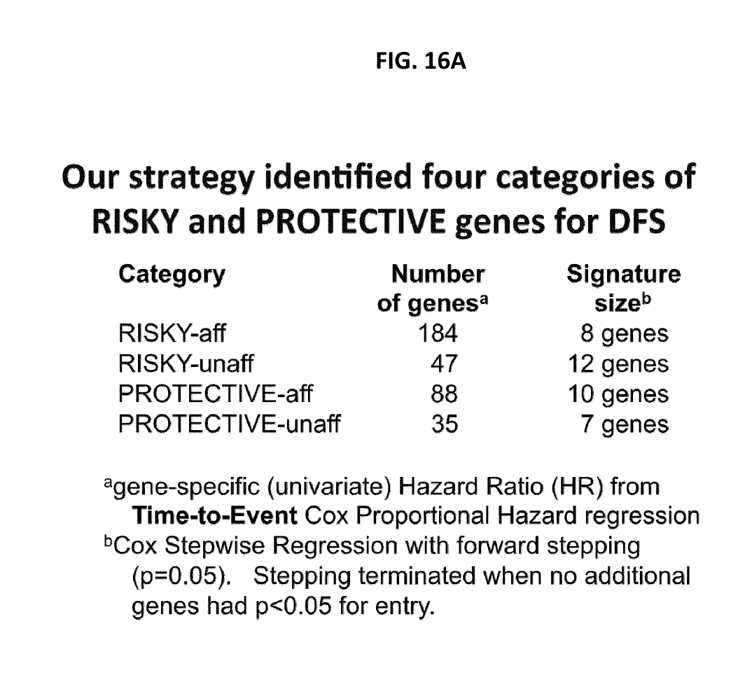

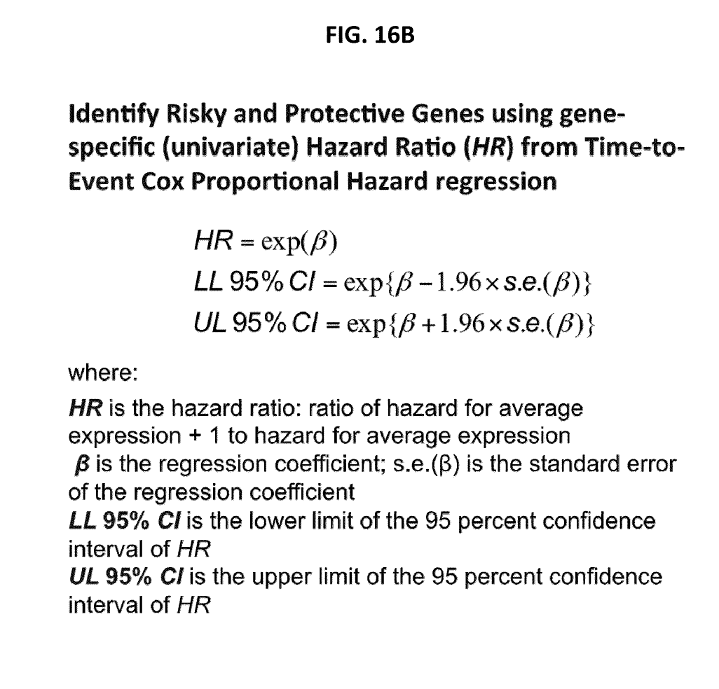

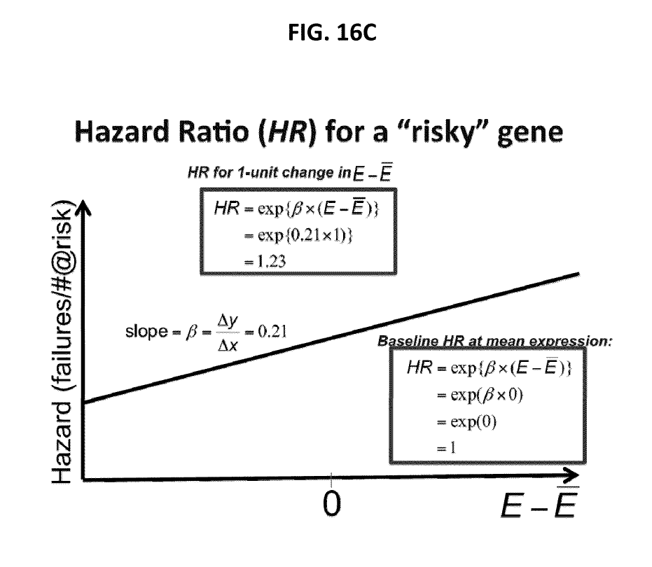

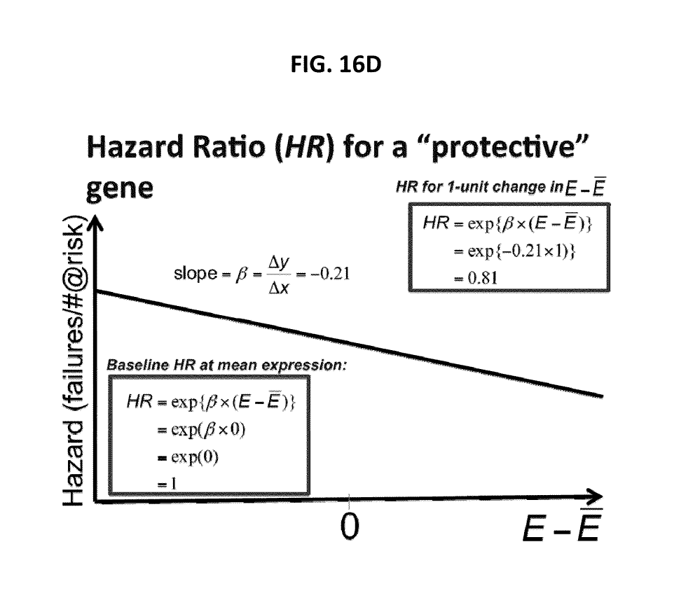

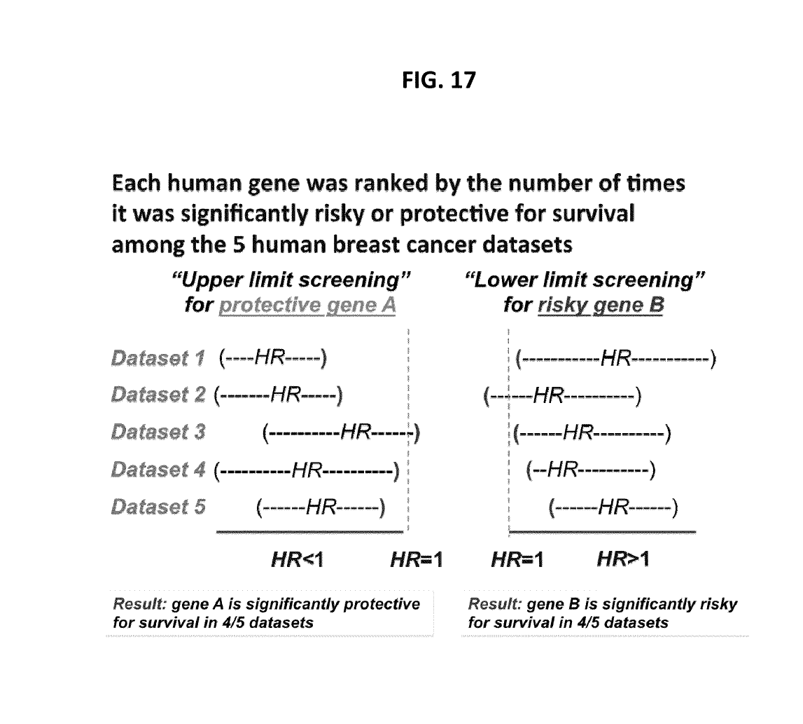

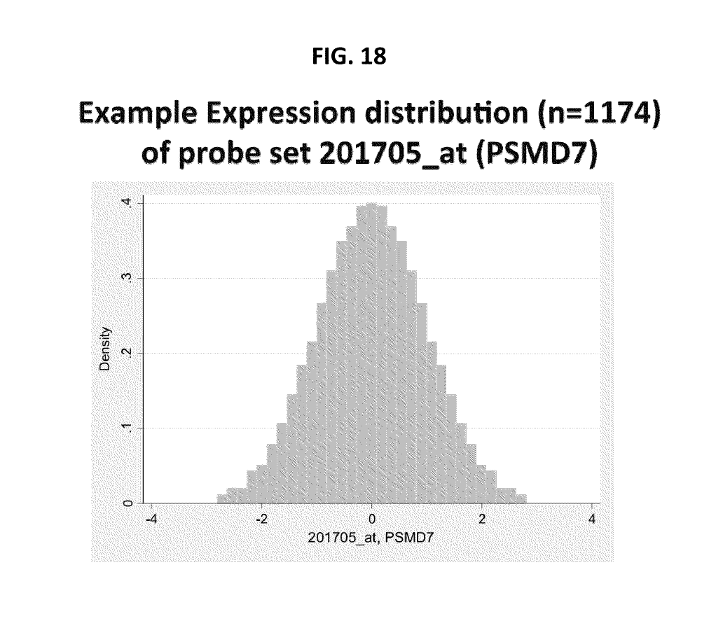

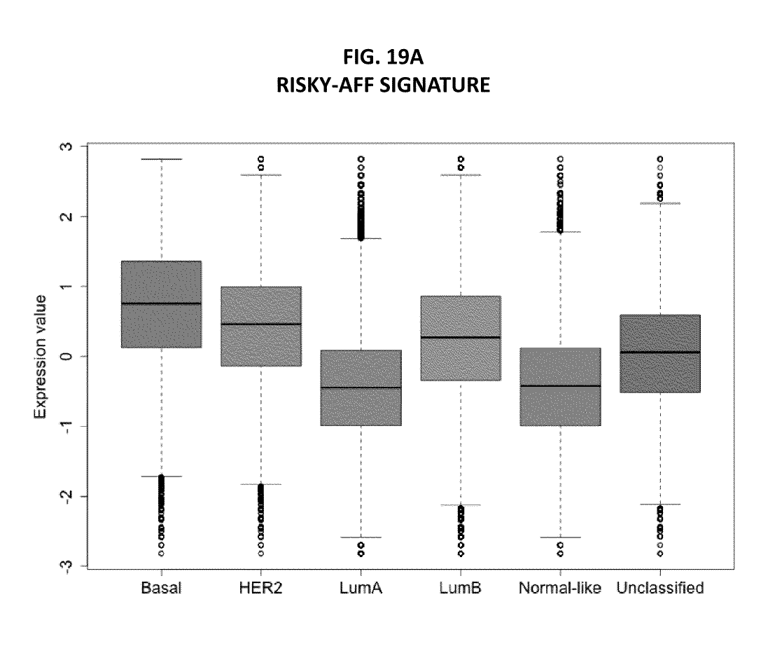

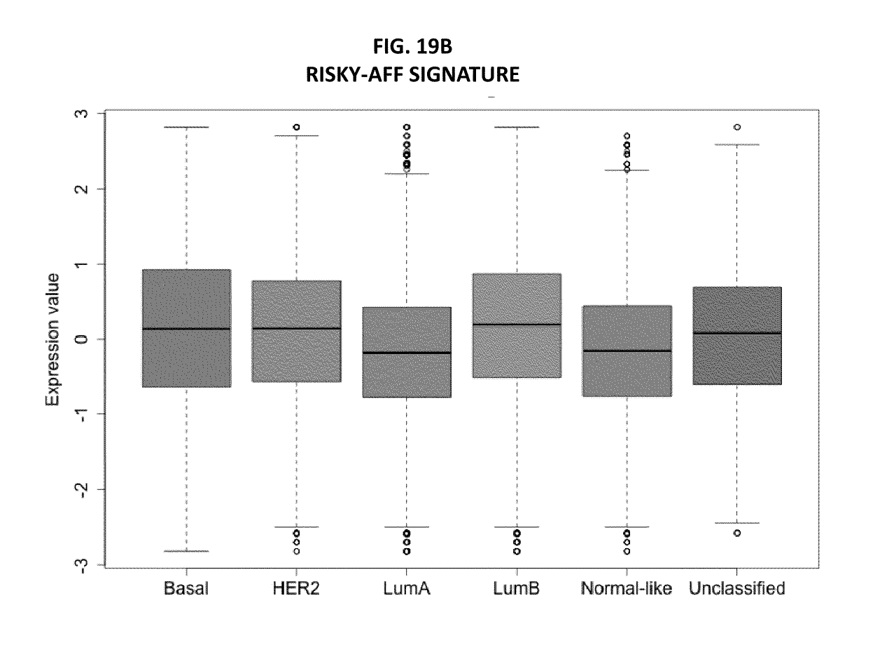

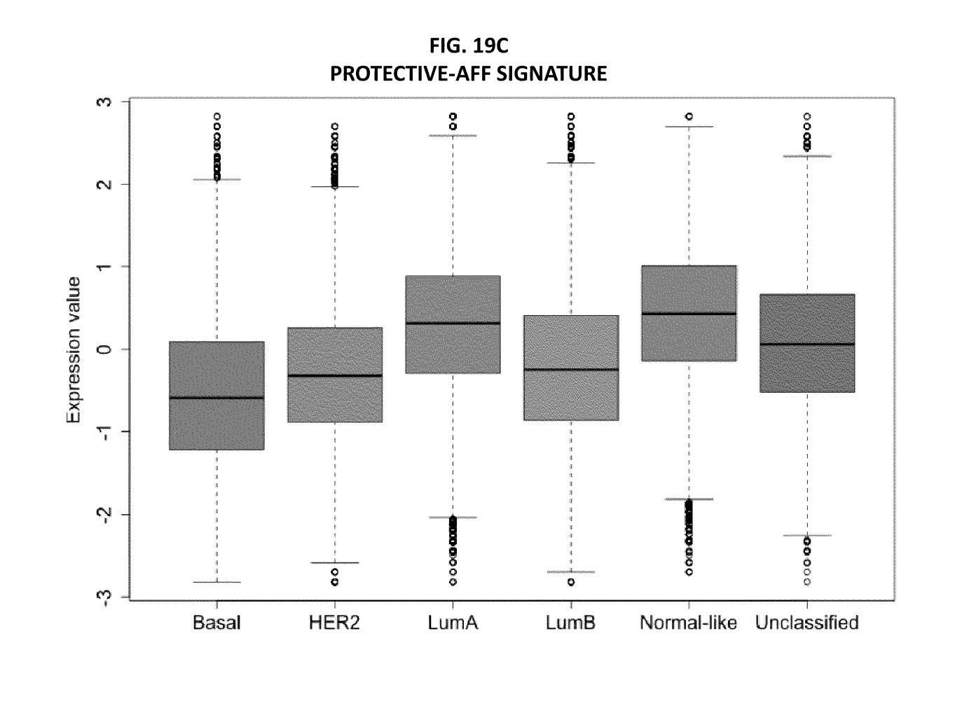

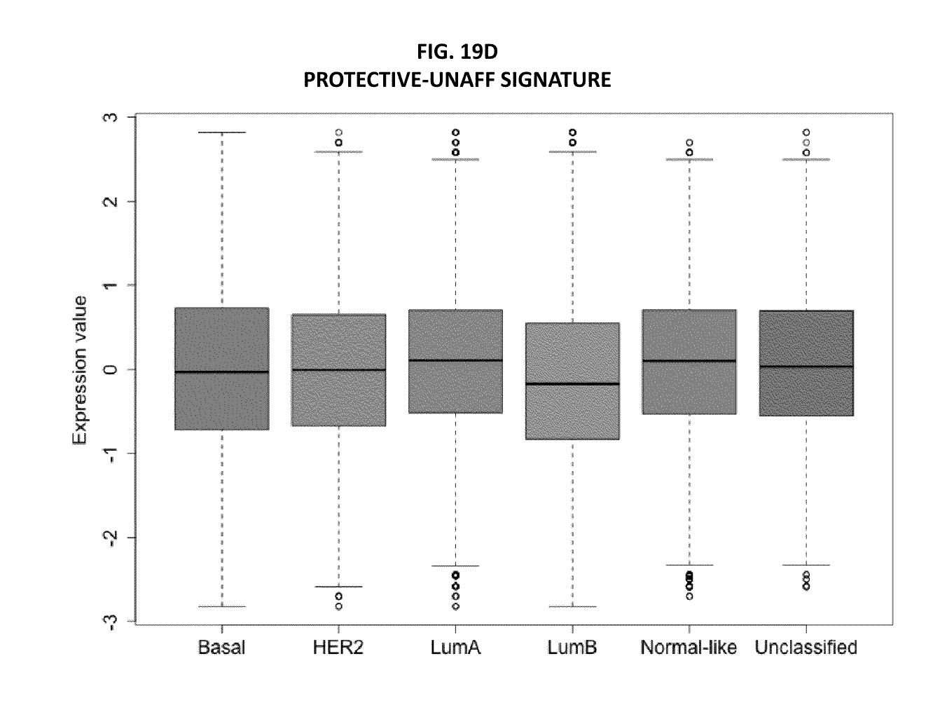

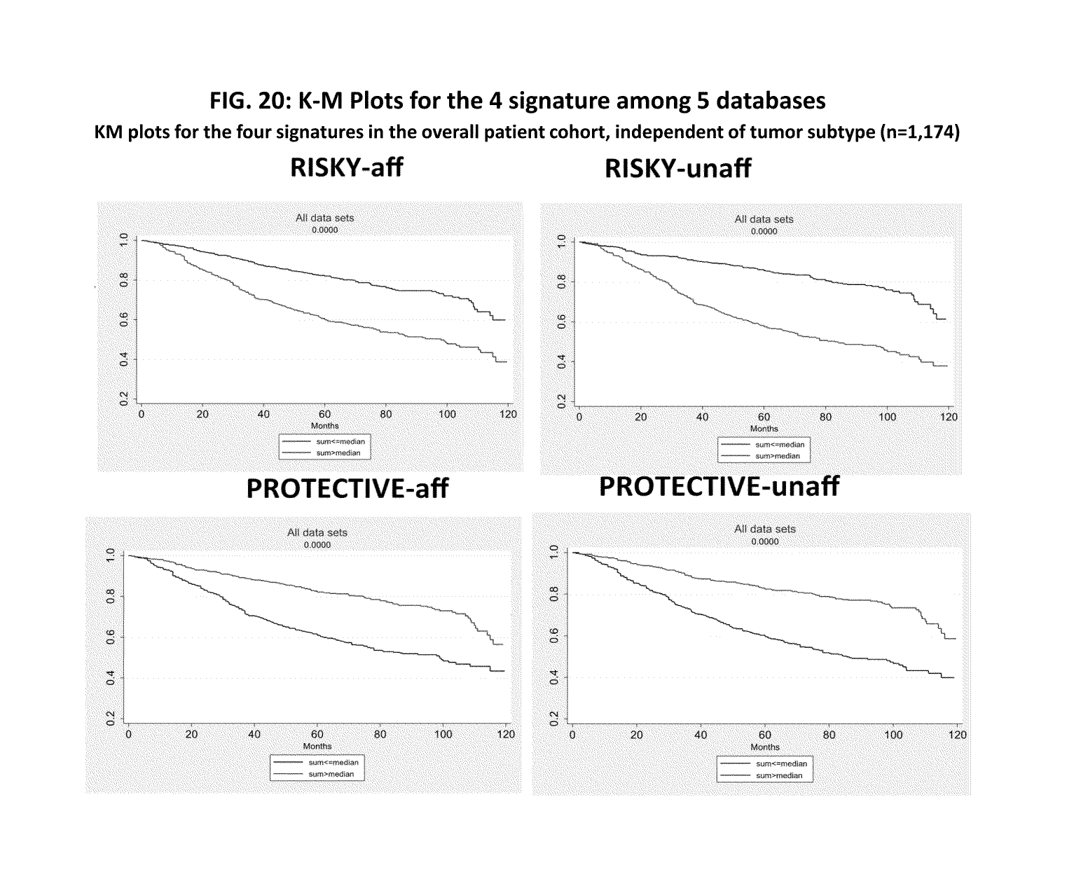

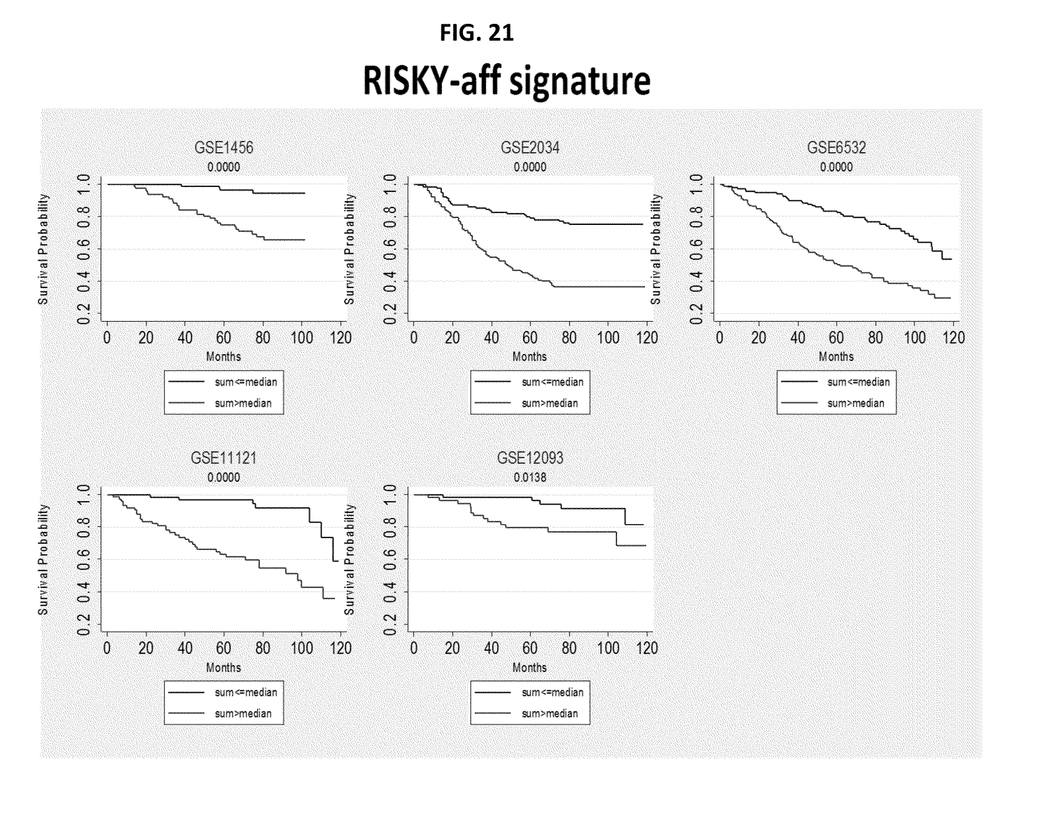

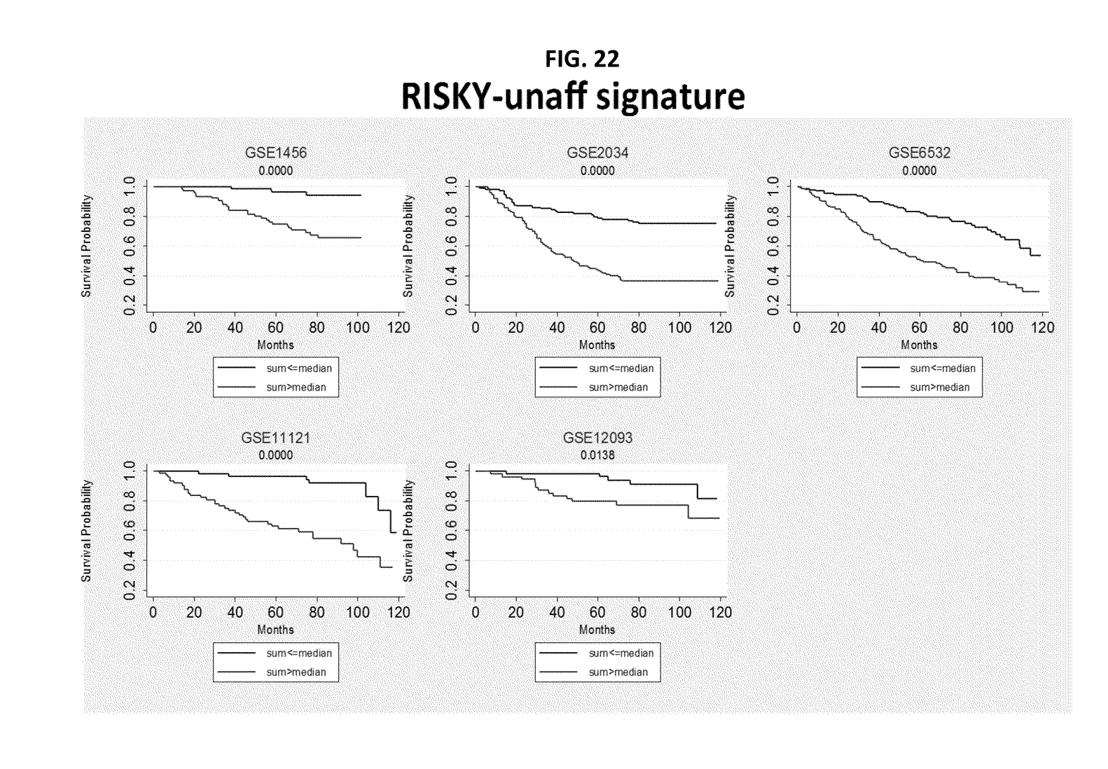

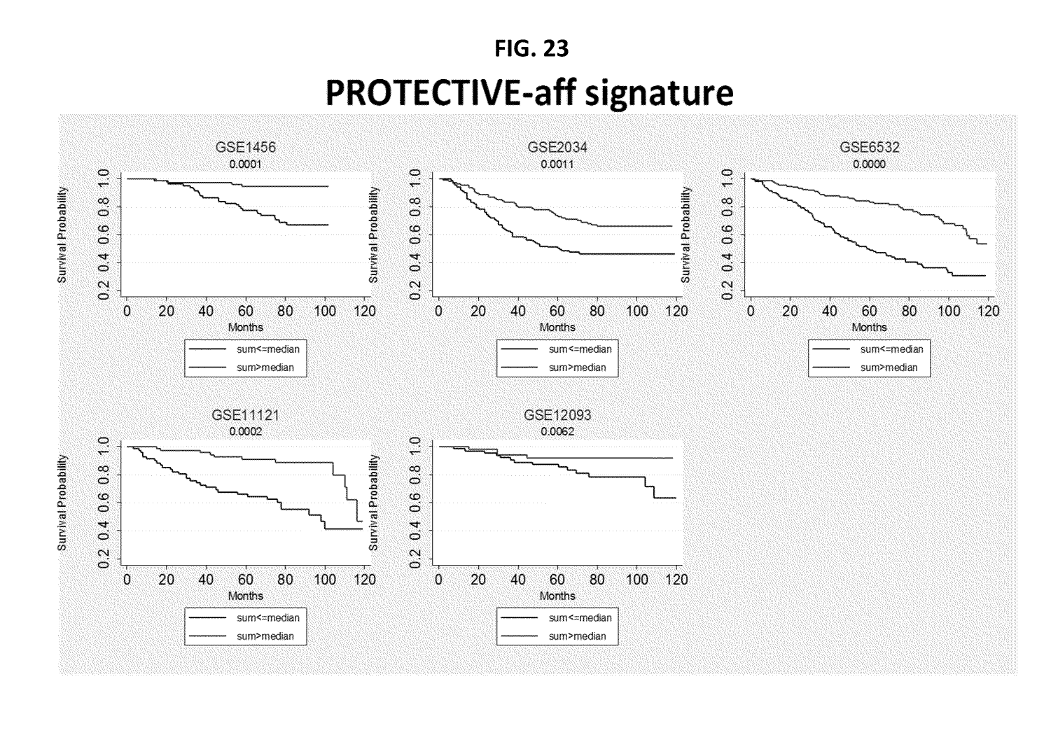

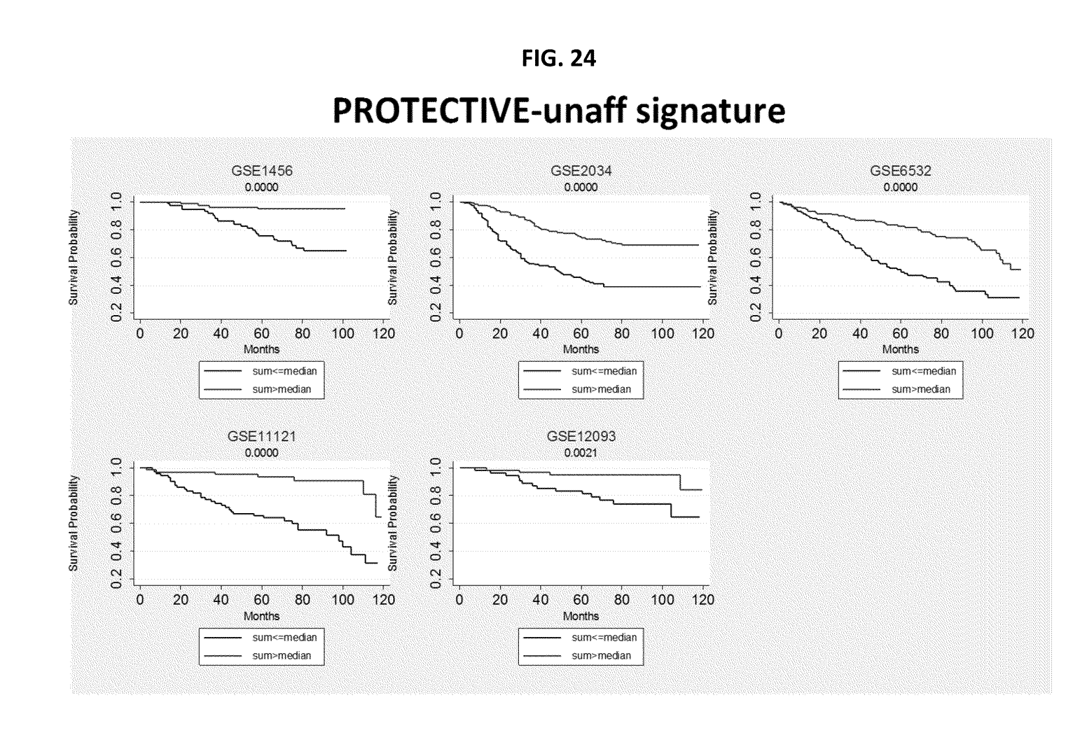

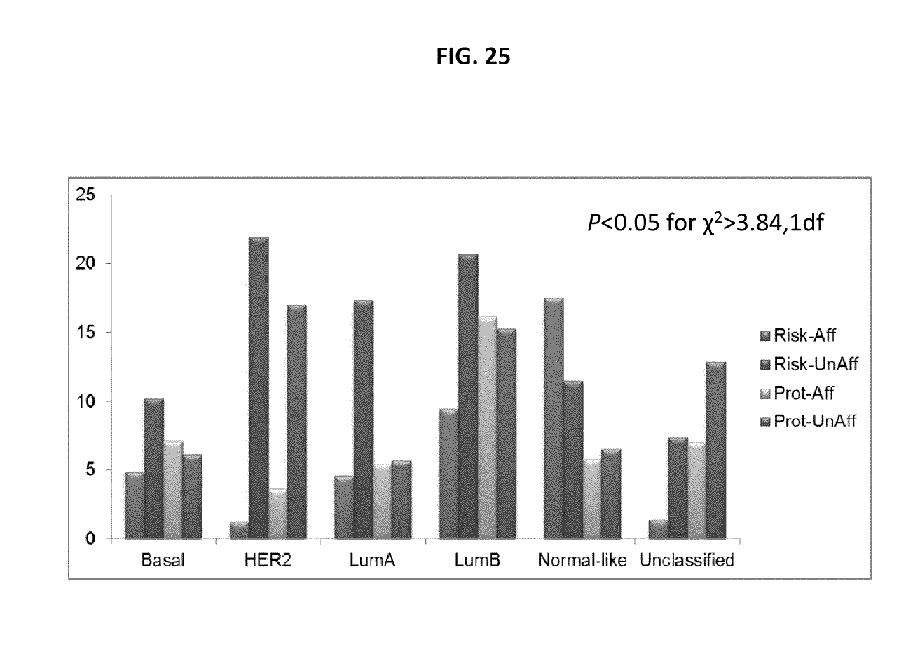

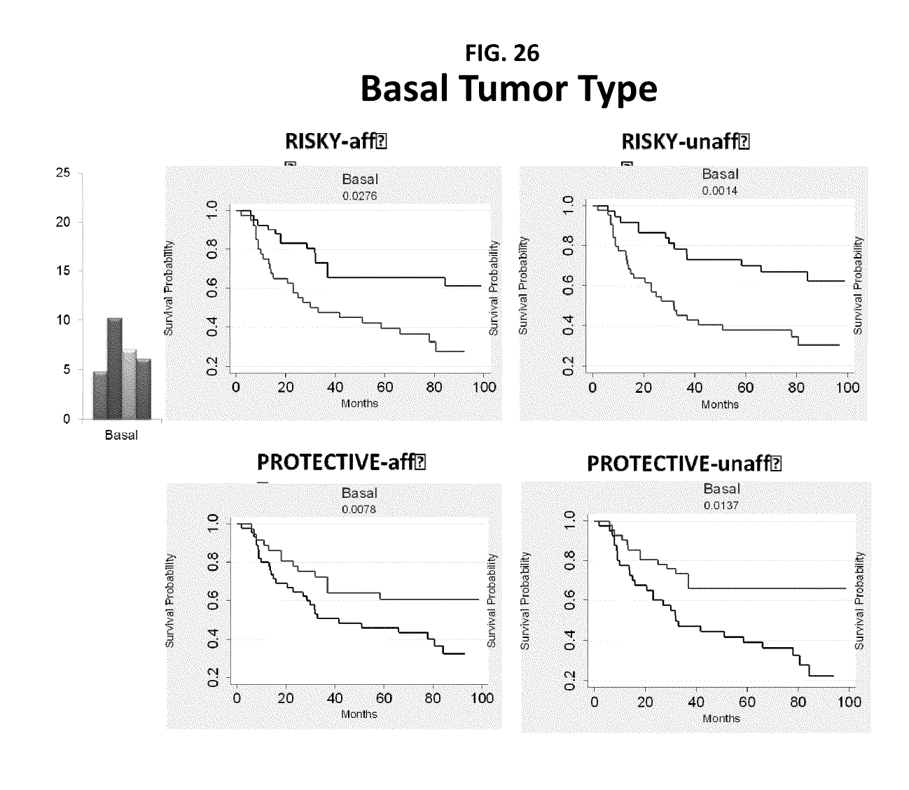

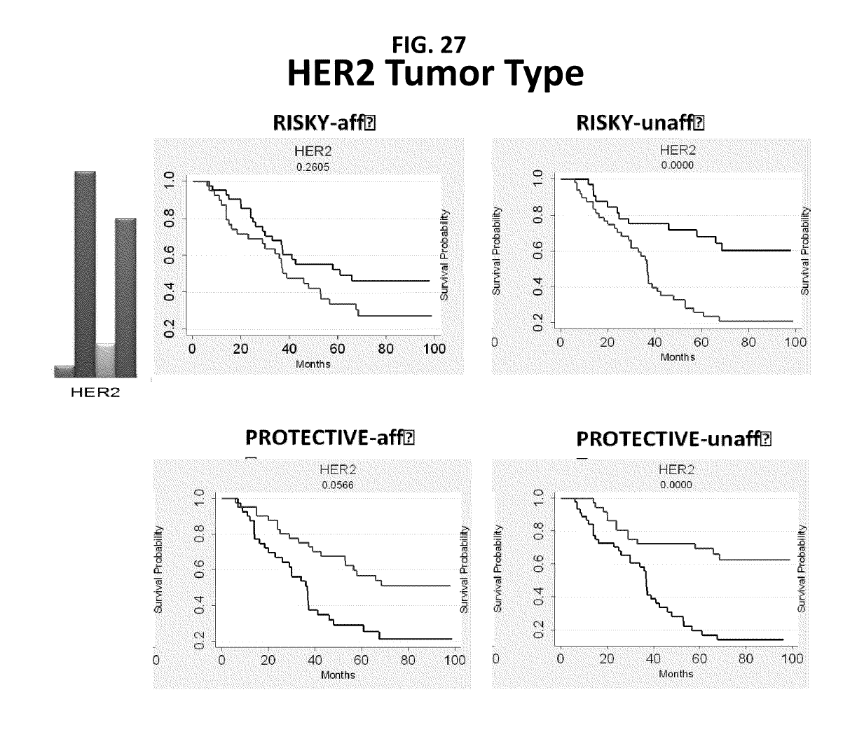

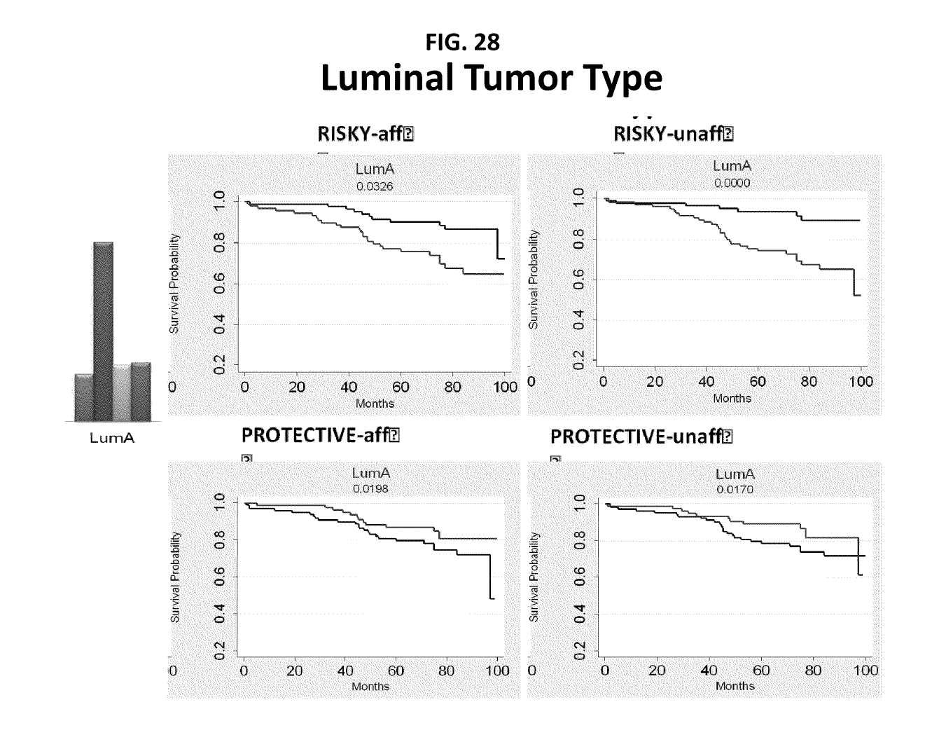

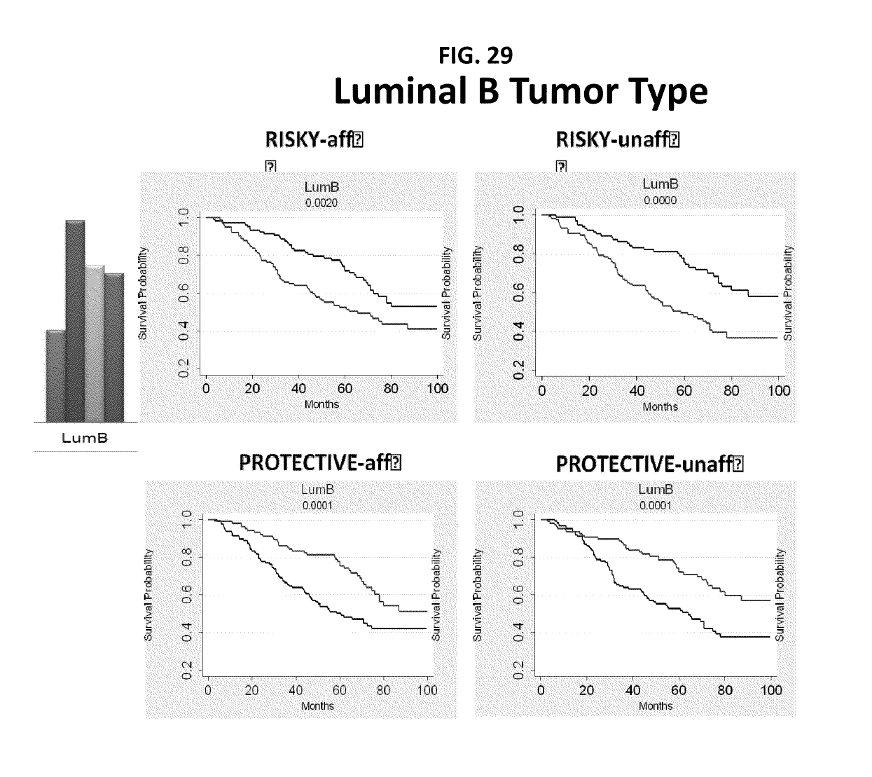

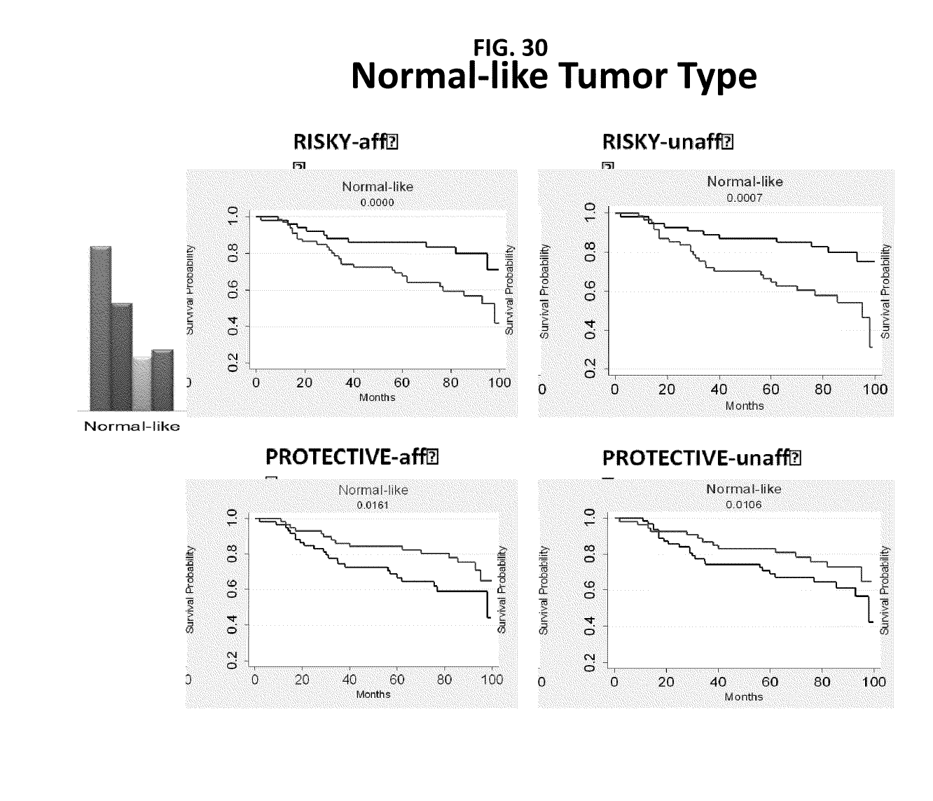

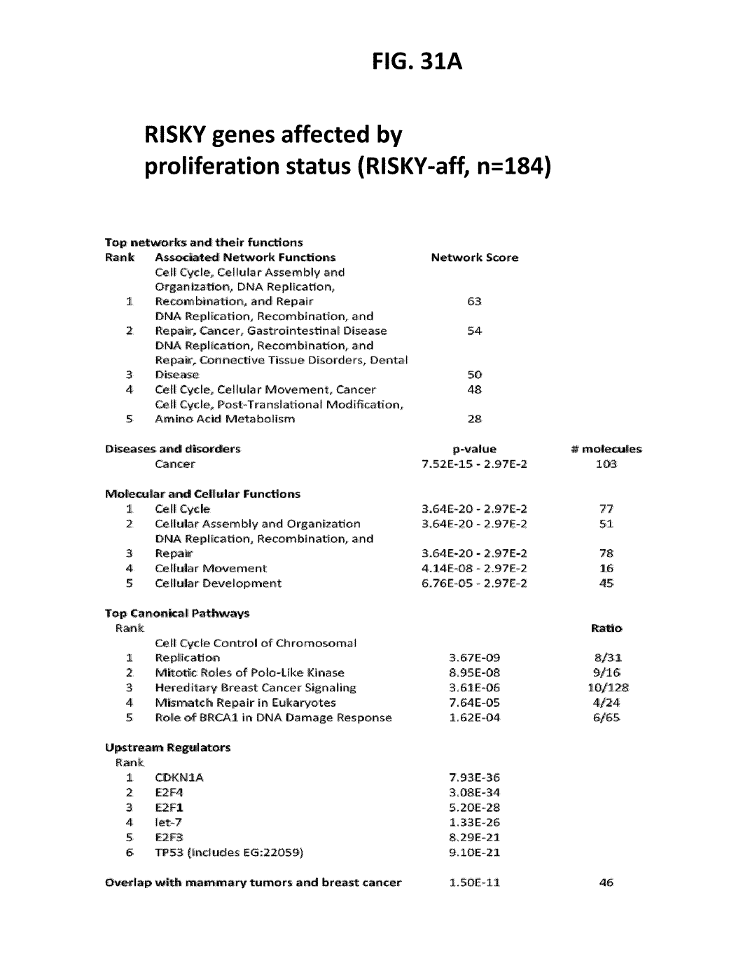

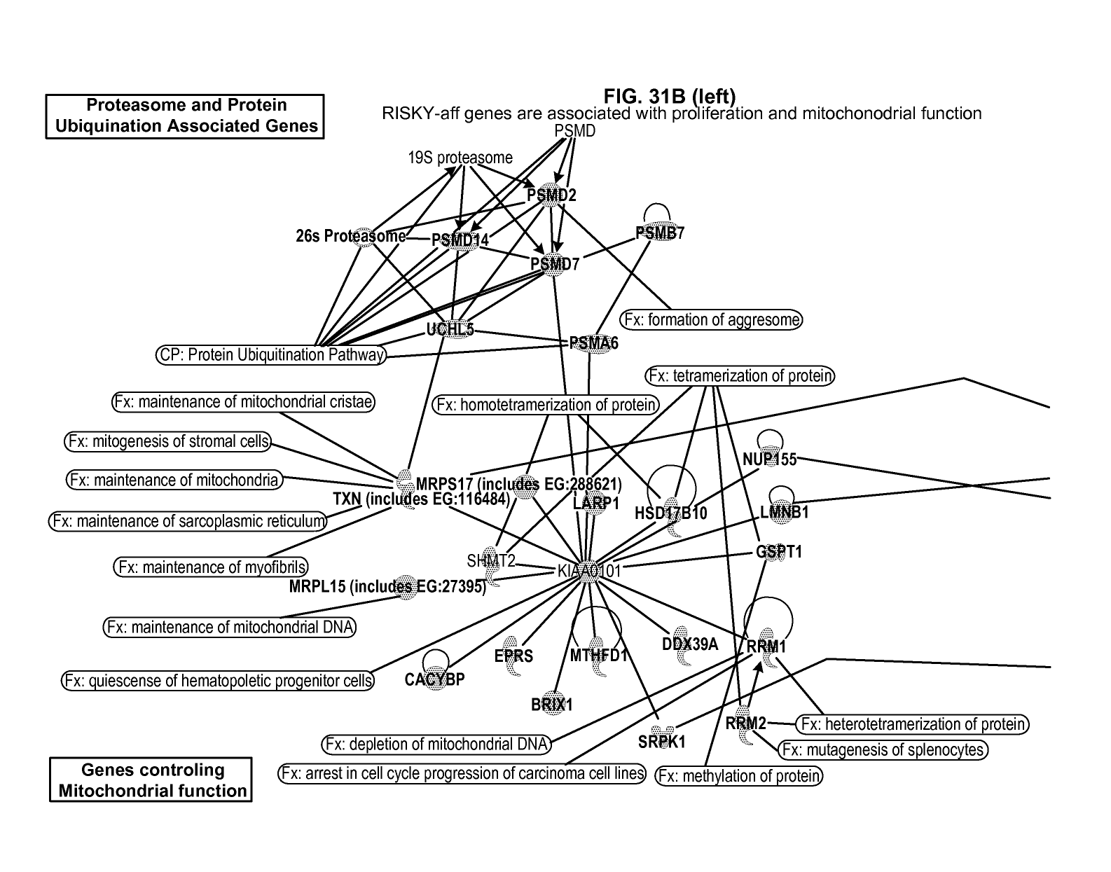

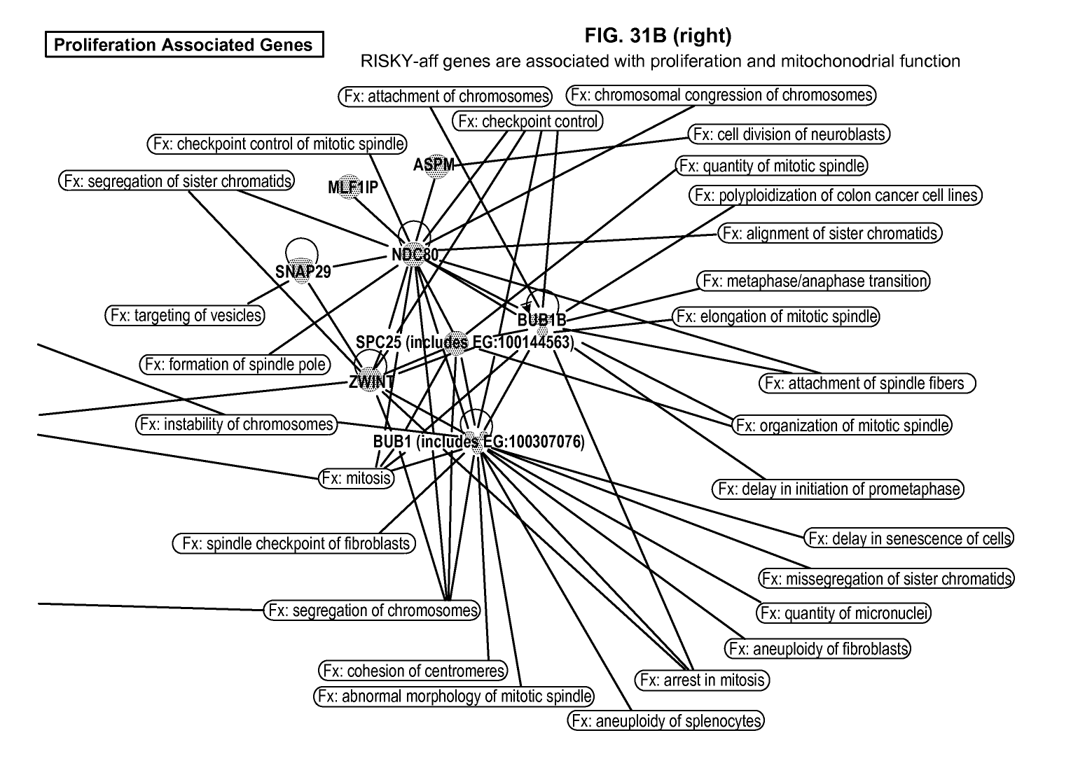

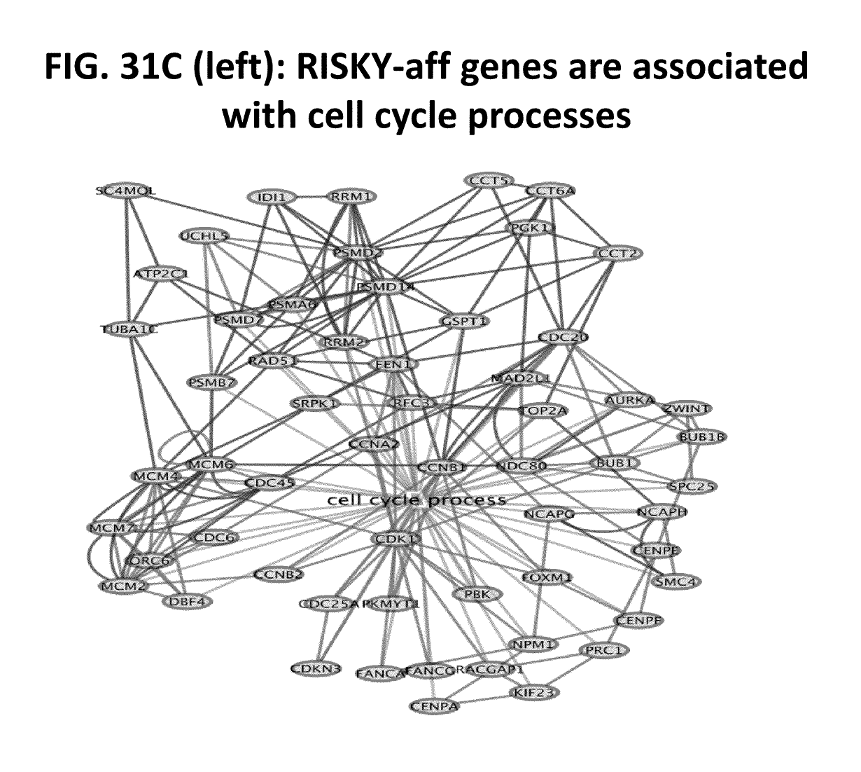

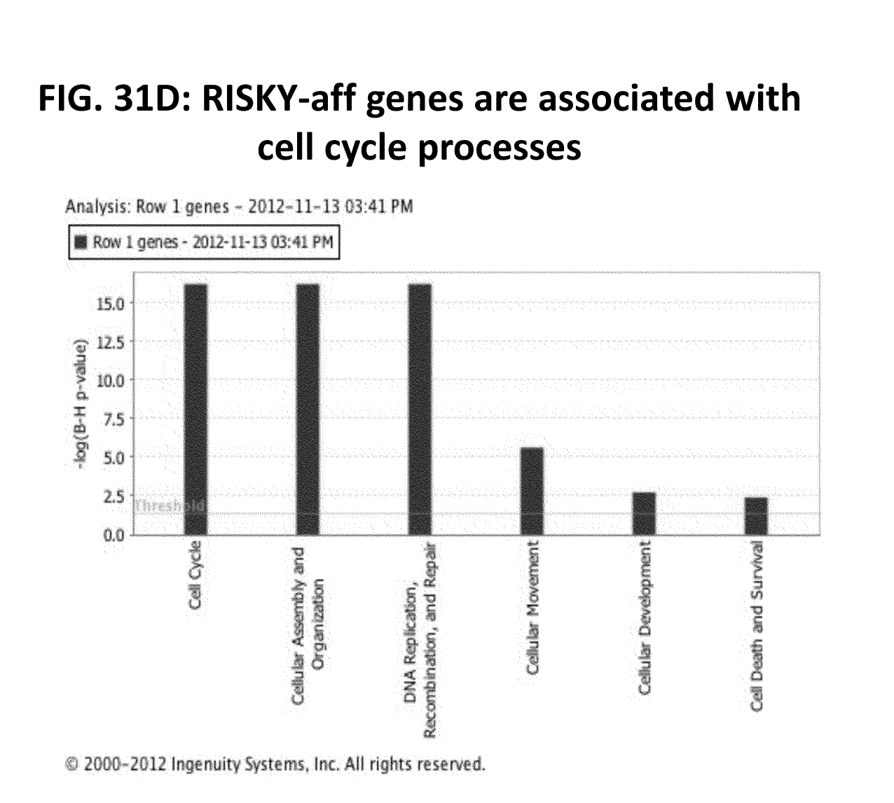

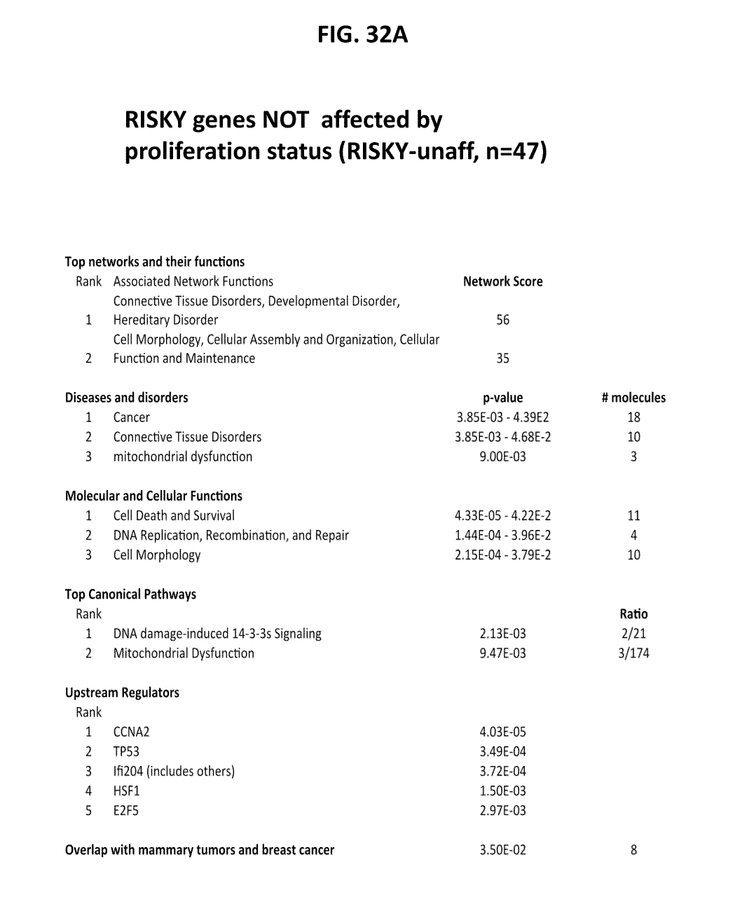

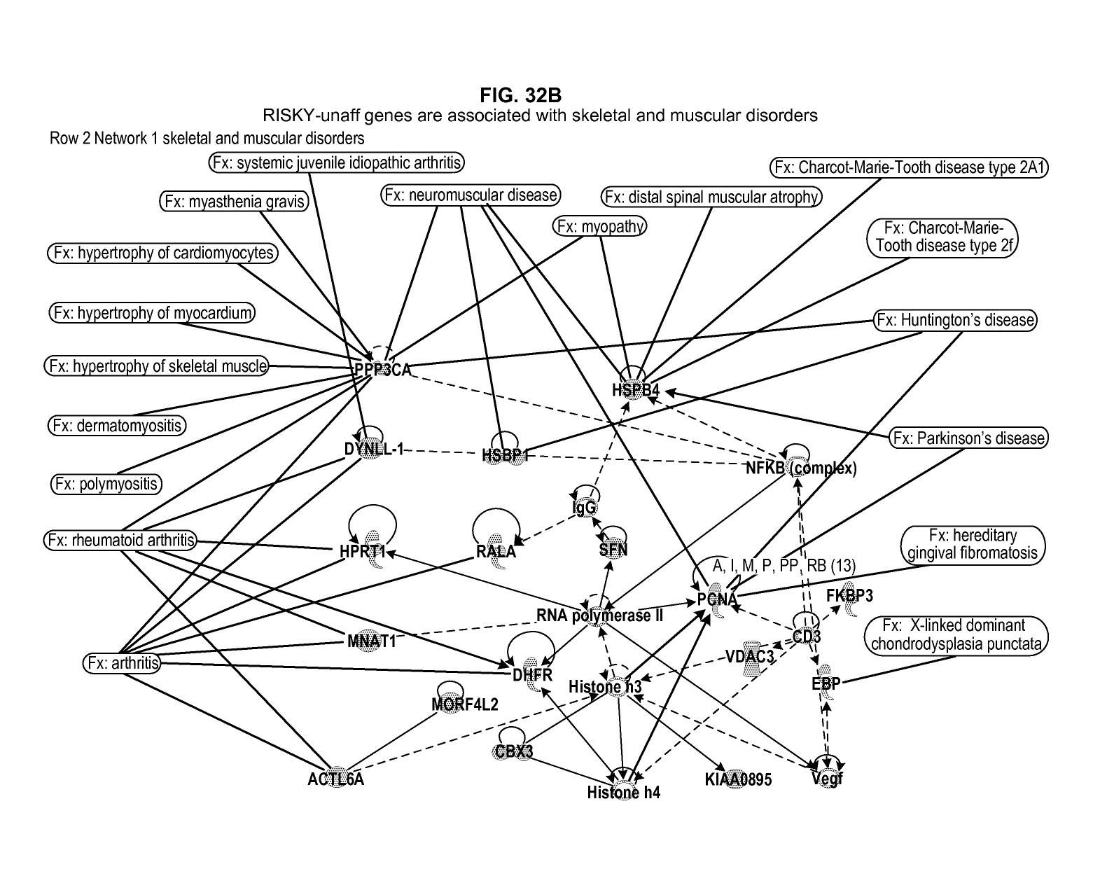

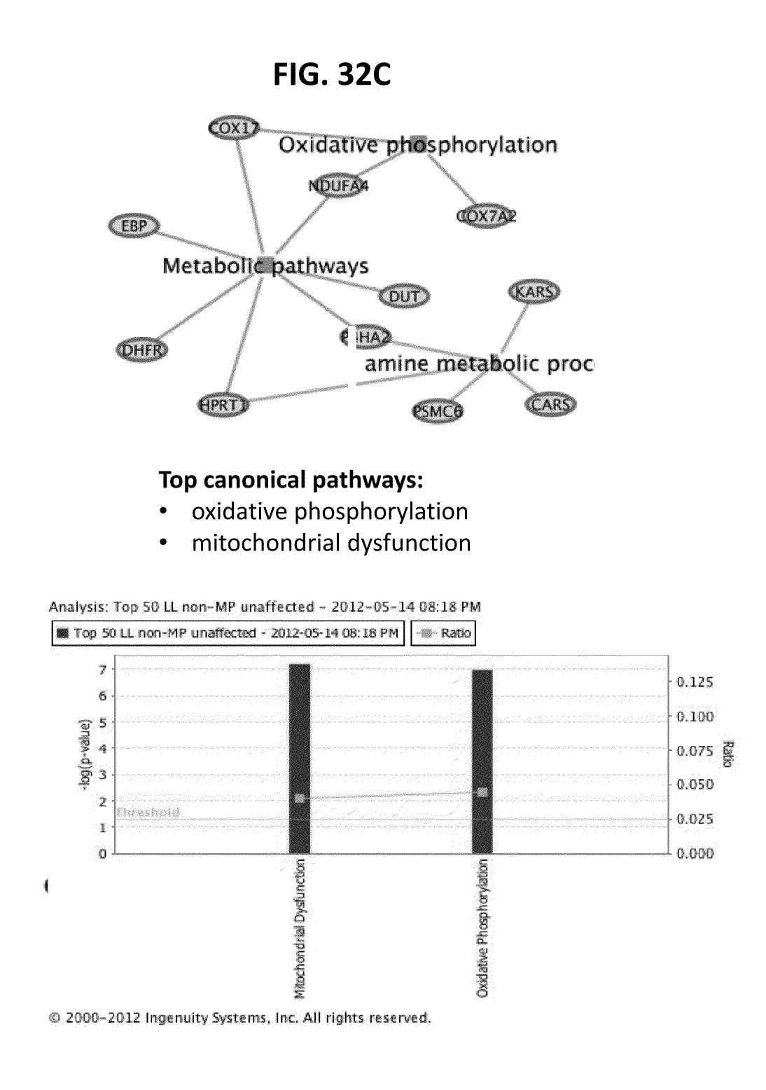

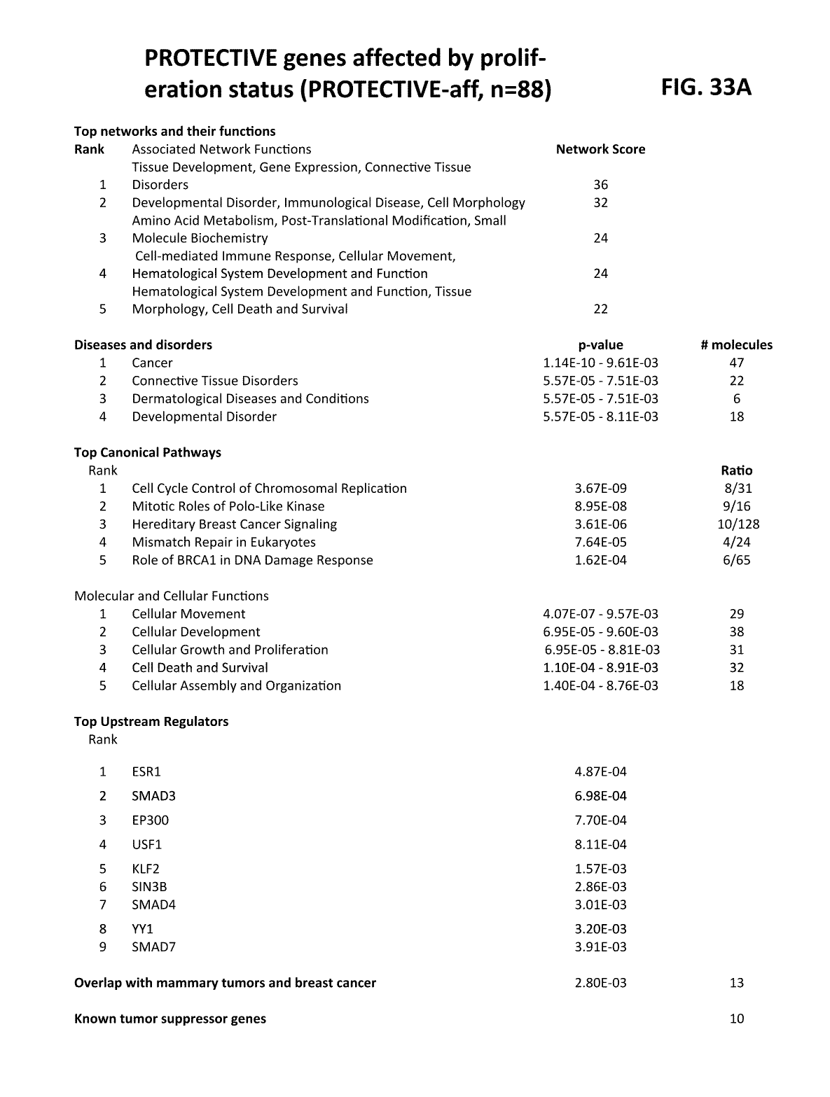

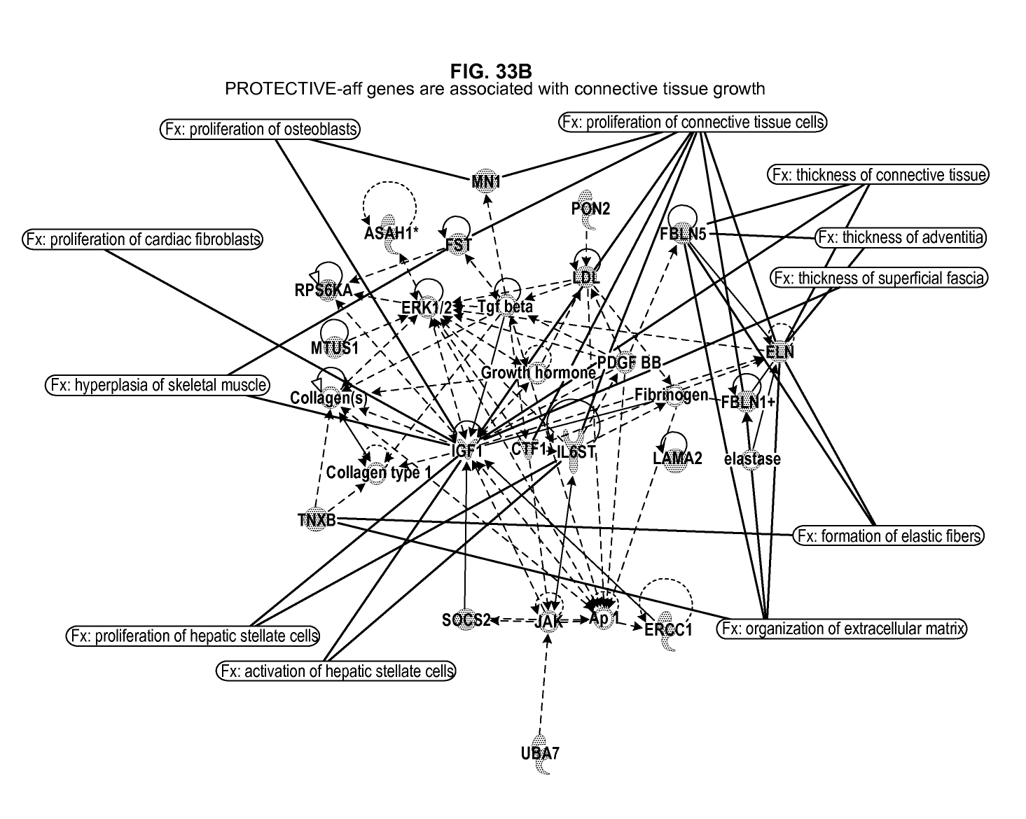

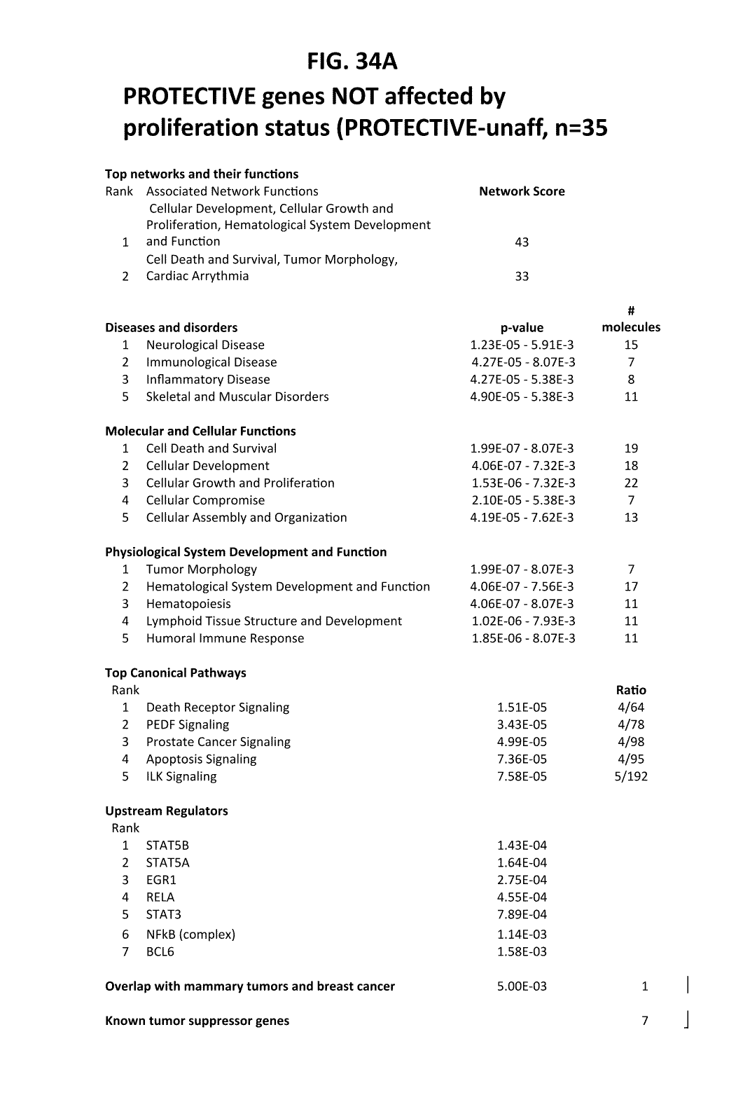

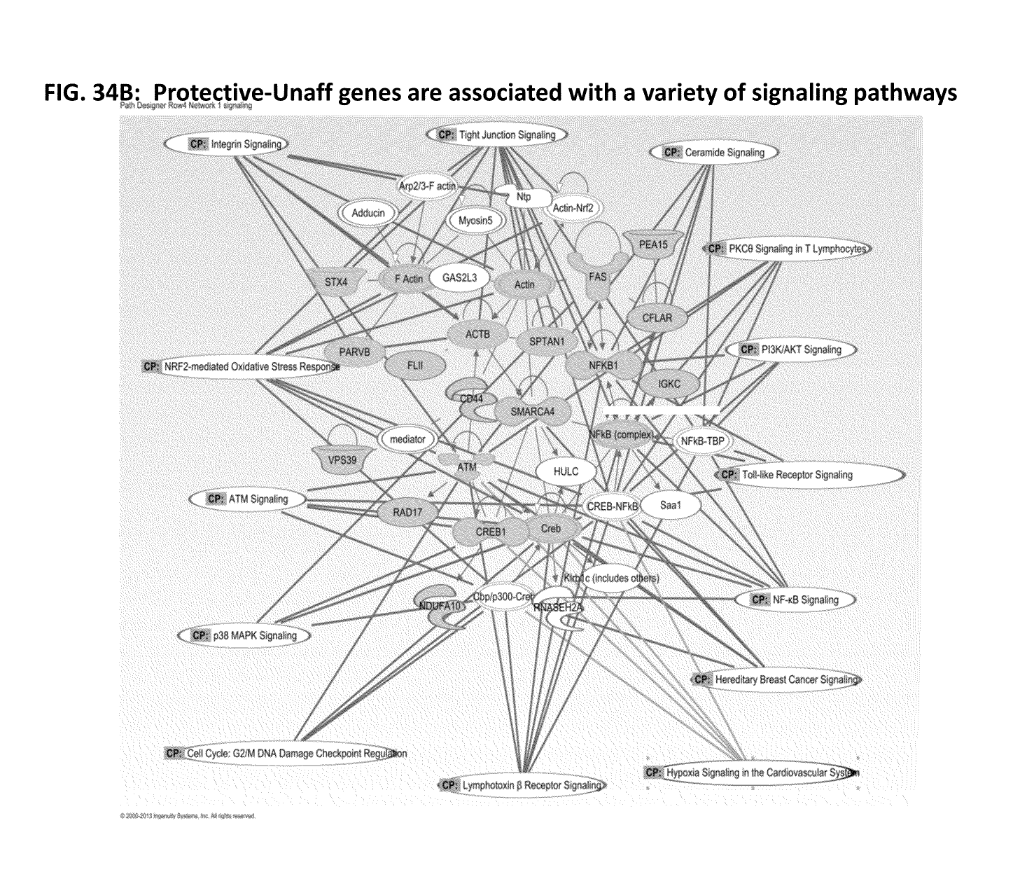

We identified Risky and Protective genes using gene-specific (univariate) Hazard Ration (HR) from Time-to-Event Cox Proportional Hazard regression as shown in FIG. 16.

Our strategy identified four categories of RISKY and PROTECTIVE genes for DFS as shown in Table 10. A Meta-PCNA Index was applied to discriminate genes that were associated with proliferation versus those that were not. We profiled microarrays of 36 tissues from normal, healthy individuals. A Meta-PCNA Index is a signature composed of the 1% genes that most positively correlated with PCNA expression across these tissues (131 genes). See the methods described by Venet D. et al. Most random gene expression signatures are significantly associated with breast cancer outcome, PLoS Comput Biol. 2011 October; 7(10):e1002240. doi: 10.1371/journal.pcbi.1002240. Epub 2011 Oct. 20; and Ge X, Yamamoto S, Tsutsumi S, Midorikawa Y, Ihara S, Wang S M, Aburatani H., Interpreting expression profiles of cancers by genome-wide survey of breadth of expression in normal tissues, Genomics. 2005 August; 86(2):127-41, both of which are hereby incorporated by reference.

Data Integration as handled as the following: (1) Merge subjects from 5 GEO breast cancer survival datasets (result [symbol] n=1174). (2) Transform time into units of month; (3) Within each data set prior to merging, for each gene (probe set), apply van Der Waerden transform on all expression values (result: standard normal, mean zero, variance unity); (4) Run stepwise Cox PH regression on filtered genes (183 lower limit, 39 upper limit) using n=1174 subjects.

Kaplan-Meier analyses of risk stratification of the four signatures was carried out as follows: The expression distributions of each component gene of the individual signatures were normalized across all women from all databases. For each signature, the expression of the component genes were added for each woman. The distribution of sums for each signature was split across the median value yielding two groups. K-M analyses was performed on each group using time to event.

It was found that total expression levels of the risky genes (Categories 1 and 2) measured above the median expression level of all the women across all the databases, indicated that a patient had shorter disease-free survival. If total risky genes expression is low, then that indicated that the patient had longer disease-free survival.

Conversely it was found that total expression levels of the protective genes (Categories 3 and 4) measured above the median expression level of all the women across all the databases, indicated that a patient had longer disease-free survival, i.e., these protective genes were protective. Likewise, if the total protective genes expression is low, then that indicated that the patient had shorter disease-free survival.

Our work is continuing to investigate other signatures for their ability to stratify variation in risk for patients within specific major breast tumor subtypes and to better understand the proliferation-independent tissue mechanisms that enhance DFS within each tumor subtype. We are also incorporating human-mouse comparative studies to identify mammary stromal and microenvironmental mechanisms that predict variation in risks for spontaneous breast cancer and sensitivity to low-dose ionizing radiation.

The above examples are provided to illustrate the invention but not to limit its scope. Other variants of the invention will be readily apparent to one of ordinary skill in the art and are encompassed by the appended claims. All references, publications, databases, and patents cited herein are hereby incorporated by reference for all purposes.

TABLE 1

| | Early (4-hr) LD responsive genes |
|---|---|
| Gene Function | in BALB/c mice (total = 313) |
| TGF-β responsive^{a} | 144 (46%) |
| MG development [19] | 89^{b} (28%) |
| Breast-cancer | 44 (14%) |
| associated [18] | |

^{a}TGF-β signaling and interaction database (http://actin.ucd.ie/tgfbeta/) and Xu et al. 2009.
^{b}42 genes overlap with TGF-β responsive genes.

TABLE 2a
COA Genes* of the Systemic Baseline Signature (n = 55)

| | | |
|---|---|---|
| Gene | Affymetrix ID | T-test |

RNA processing genes

| | | |
|---|---|---|
| MAGOHB | 218894_s_at | 7.39E−05 |
| PAPOLA | 212720_at | 8.98E−06 |
| PNPT1 | 225291_at | 5.46E−10 |
| POP4 | 202868_s_at | 1.99E−03 |
| PPIH | 204228_at | 1.77E−05 |
| RBM39 | 208720_s_at | 9.41E−05 |
| RPS6 | 209134_s_at | 4.64E−03 |
| SUPT16H | 233827_s_at | 7.69E−05 |
| TXNL4A | 202836_s_at | 5.81E−07 |

Stress response genes

| | | |
|---|---|---|
| EIF2S1 | 201144_s_at | 1.75E−07 |
| GNA13 | 224761_at | 7.16E−09 |
| GNB1 | 200744_s_at | 8.14E−05 |
| HLA-DRA | 208894_at | 2.33E−05 |
| PRDX2 | 215067_x_at | 3.39E−04 |
| PRDX3 | 209766_at | 4.39E−03 |
| RAD23A | 201039_s_at | 2.33E−04 |
| RPS6 | 209134_s_at | 4.64E−03 |
| RUNX1 | 210365_at | 4.85E−06 |
| SMC6 | 218781_at | 2.95E−05 |
| SUPT16H | 233827_s_at | 7.69E−05 |

Other genes

| | | |
|---|---|---|
| ABCB10 | 223320_s_at | 3.50E−03 |
| ABCF1 | 200045_at | 1.04E−09 |
| BAT5 | 224756_s_at | 2.01E−06 |
| BMP2K | 37170_at | 6.77E−03 |
| C17orf95 | 225808_at | 5.28E−08 |
| C19orf56 | 217780_at | 1.64E−03 |
| C5orf22 | 203738_at | 5.40E−03 |
| CAP1 | 213798_s_at | 7.70E−06 |
| CBX7 | 212914_at | 2.68E−04 |
| CHCHD3 | 217972_at | 5.68E−07 |
| CHCHD4 | 229595_at | 1.90E−05 |
| CLASP2 | 212308_at | 8.48E−03 |
| CLDND1 | 208925_at | 1.06E−04 |
| DDX19A | 202578_s_at | 1.52E−05 |
| DNAJC10 | 221781_s_at | 9.41E−12 |
| GADD45GIP1 | 212891_s_at | 2.13E−03 |
| GBP1 | 202270_at | 1.17E−05 |
| HLA-B | 208729_x_at | 9.92E−06 |
| KIF5B | 224662_at | 2.39E−05 |
| MCART1 | 232092_at | 1.86E−06 |
| MCM6 | 201930_at | 1.79E−07 |
| MTFR1 | 203207_s_at | 1.79E−07 |
| NRD1 | 208709_s_at | 4.64E−08 |
| PDK1 | 206686_at | 5.81E−06 |
| PDXDC1 | 212053_at | 1.30E−05 |
| PEBP1 | 211941_s_at | 9.61E−05 |
| PHF20 | 235389_at | 1.62E−03 |
| PI4K2B | 222631_at | 1.87E−11 |
| PIGO | 209998_at | 5.92E−03 |
| PPME1 | 217841_s_at | 2.47E−04 |
| RAB6B | 221792_at | 1.08E−04 |
| SAPS3 | 222467_s_at | 3.76E−06 |
| SCAND1 | 231059_x_at | 3.21E−08 |
| SHC1 | 201469_s_at | 2.47E−03 |
| SLC15A2 | 240159_at | 6.86E−03 |
| SNX6 | 222410_s_at | 5.35E−12 |
| TM2D2 | 224413_s_at | 3.83E−08 |

TABLE 2b
COA Genes* of the BALB/c 1 month LD Signature (n = 36)

| | | |
|---|---|---|
| Gene | Affymetrix ID | T-test |

Mitotic genes

| | | |
|---|---|---|
| CAD | 202715_at | 2.63E−03 |
| CCNK | 225824_at | 2.07E−03 |
| CDC7 | 204510_at | 4.27E−06 |
| CDT1 | 228868_x_at | 1.21E−08 |
| CENPH | 231772_x_at | 1.45E−04 |
| CHEK1 | 205393_s_at | 6.03E−09 |
| EZH2 | 203358_s_at | 8.18E−12 |
| GINS1 | 206102_at | 2.95E−07 |
| HELLS | 220085_at | 3.39E−04 |
| MCM2 | 202107_s_at | 8.02E−07 |
| MCM3 | 201555_at | 6.17E−05 |
| MCM4 | 212141_at | 5.50E−07 |
| MCM5 | 201755_at | 4.47E−09 |
| MCM6 | 201930_at | 9.09E−13 |
| MCM7 | 208795_s_at | 2.14E−05 |
| MYC | 202431_s_at | 2.92E−03 |
| POLD1 | 203422_at | 1.51E−04 |
| PRIM1 | 205053_at | 9.19E−03 |
| RFC5 | 203209_at | 5.46E−04 |
| RRM2 | 201890_at | 1.74E−13 |
| SNRPD3 | 202567_at | 1.18E−06 |
| TK1 | 202338_at | 1.14E−11 |
| TYMS | 202589_at | 3.61E−09 |
| UHRF1 | 225655_at | 3.07E−10 |
| WDHD1 | 216228_s_at | 2.34E−06 |

Other genes

| | | |
|---|---|---|
| CCDC86 | 203119_at | 1.05E−05 |
| ELOVL6 | 204256_at | 8.42E−04 |
| GABRP | 205044_at | 5.67E−06 |
| KRT17 | 205157_s_at | 5.87E−03 |
| MMP12 | 204580_at | 2.68E−05 |
| NUP107 | 218768_at | 5.26E−04 |
| NUTF2 | 202397_at | 1.34E−04 |
| PA2G4 | 208676_s_at | 3.67E−03 |
| SLC7A5 | 201195_s_at | 5.40E−14 |
| SQLE | 209218_at | 1.45E−06 |
| WASF1 | 204165_at | 6.87E−03 |

TABLE 3

| | BALB/c | BALB/c | C57BI/6 | C57BI/6 |
|---|---|---|---|---|
| Gene | Low¹ | High¹ | Low² | High¹ |
| Hist1h2ad | −1.23 | −1.84 | Ns | −1.25 |
| Mpeg1 | −1.18 | −1.13 | Ns | −1.13 |
| Fbn2 | −1.11 | −1.66 | Ns | −1.08 |
| H19 | −1.01 | −1.56 | Ns | −1.68 |
| Cdt1 | −0.94 | −1.14 | Ns | −0.99 |
| Clec7a | −0.92 | −1.41 | Ns | −1.19 |
| Mcm5 | −0.89 | −1.15 | Ns | −0.60 |
| Irf8 | −0.87 | −1.06 | Ns | −0.71 |
| Cdt1 | −0.85 | −1.11 | ns | −0.75 |
| Stmn1 | −0.84 | −1.48 | ns | −1.11 |
| Lst1 | −0.81 | −1.12 | ns | −0.58 |
| Uhrf1 | −0.71 | −1.13 | ns | −0.84 |
| Col9a1 | −0.71 | −1.82 | ns | −0.89 |
| Irf8 | −0.70 | −1.53 | ns | −1.07 |
| Fyb | −0.69 | −1.04 | ns | −1.02 |
| Tlr1 | −0.69 | −0.72 | ns | −0.60 |
| Mcm6 | −0.69 | −1.02 | ns | −1.18 |
| Gzma | −0.68 | −1.08 | ns | −0.89 |
| Cybb | −0.66 | −0.98 | ns | −0.66 |
| Ptprc | −0.64 | −1.21 | ns | −1.00 |
| Ccl5 | −0.61 | −0.79 | ns | −0.91 |
| Copg2as2 | 0.65 | 0.85 | ns | 0.82 |
| Fbxo21 | 0.66 | 0.62 | ns | 1.08 |
| Anxa8 | 0.69 | 1.04 | ns | 0.63 |
| Zbtb16 | 0.70 | 1.13 | ns | 0.90 |
| Zbtb16 | 0.76 | 1.13 | ns | 0.74 |
| Cdh13 | 0.96 | 0.84 | ns | 0.63 |
| Zbtb16 | 1.19 | 1.46 | ns | 1.10 |

¹Fold change (log₂) with respect to sham irradiated animals (p < 8.7E−02)
²ns - not significantly modulated after low dose with respect to sham

TABLE 4

| | Time^{b} | C57BL/6 | BALB/c | p-value |
|---|---|---|---|---|
| Micronucleated RET^{a} | −1 day | 0.27 ± 0.03 | 0.33 ± 0.05 | |
| | 6 days | 0.22 ± 0.02 | 0.34 ± 0.05 | |
| | 28 days | 0.25 ± 0.05 | 0.37 ± 0.08 | |
| | average | 0.25 | 0.34 | <0.0001 |
| Micronucleated NCE^{a} | −1 day | 0.14 ± 0.01 | 0.23 ± 0.02 | |
| | 6 days | 0.14 ± 0.01 | 0.23 ± 0.02 | |
| | 28 days | 0.13 ± 0.00 | 0.20 ± 0.01 | |
| | average | 0.14 | 0.22 | <0.0001 |

^{a}Percent ± standard deviation
^{b}Time in relation to 4^{th} sham

TABLE 5

| Dose (cGy)^{a} | Time (days)^{b} | Mice n | Total RET | MN-RET (% ± S.D.) | Total NCE | MN-NCE (% ± S.D.) |
|---|---|---|---|---|---|---|
| sham (0 Gy) | | 6^{c} | 359,113 | 887 (0.25 ± 0.02) | 15,076,138 | 20,390 (0.14 ± 0.004) |
| 3 × 7.5 | −1 | 6 | 119,649 | 351 (0.30 ± 0.03)^{f} | 4,525,395 | 7,519 (0.17 ± 0.02)^{d} |
| 3 × 180 | −1 | 6 | 118,656 | 1,344 (1.12 ± 0.28)^{d} | 2,273,975 | 7,983 (0.35 ± 0.02)^{d} |
| 3 × (7.5 + 180) | −1 | 6 | 118,685 | 1,315 (1.10 ± 0.28)^{d} | 1,656,437 | 6,015 (0.36 ± 0.04)^{d} |
| 4 × 7.5 | 6 | 6 | 119,684 | 316 (0.27 ± 0.03) | 5,444,232 | 9,097 (0.17 ± 0.01)^{d} |
| 4 × 180 | 6 | 6 | 118,801 | 1,199 (1.00 ± 0.14)^{d} | 573,214 | 3,160 (0.55 ± 0.02)^{d,e} |
| 4 × (7.5 + 180) | 6 | 6 | 118,824 | 1,176 (0.98 ± 0.22)^{d} | 793,657 | 4,773 (0.60 ± 0.07)^{d,e} |
| 4 × 7.5 | 28 | 5 | 99,725 | 275 (0.28 ± 0.06) | 4,719,142 | 6,896 (0.14 ± 0.02) |
| 4 × 180 | 28 | 6 | 119,643 | 357 (0.30 ± 0.09) | 3,535,137 | 10,692 (0.30 ± 0.03)^{d} |
| 4 × (7.5 + 180) | 28 | 6 | 119,605 | 395 (0.33 ± 0.08) | 3,064,364 | 9,638 (0.32 ± 0.05)^{d} |

^{a}Once a week with 6 hr of separation for multiple daily doses
^{b}In relation to 4th irradiation
^{c}For each mouse, an overall average was obtained by pooling the values from the tree collection points
^{d}P < 0.0001 vs sham
^{e}P < 0.0001 vs 3 weeks

TABLE 6

| Dose (cGy)^{a} | Time (days)^{b} | Mice n | Total RET | MN-RET (% ± S.D.) | Total NCE | MN-NCE (% ± S.D.) |
|---|---|---|---|---|---|---|
| sham (0 Gy) | | 6^{c} | 318,926 | 1,074 (0.34 ± 0.04) | 14,582,445 | 32,750 (0.22 ± 0.01) |
| 3 × 7.5 | −1 | 6 | 119,578 | 422 (0.35 ± 0.03) | 5,242,933 | 11,578 (0.25 ± 0.02)^{f} |
| 3 × 180 | −1 | 6 | 118,251 | 1,749 (1.46 ± 0.22)^{d} | 2,113,742 | 11,933 (0.57 ± 0.04)^{d} |
| 3 × (7.5 + 180) | −1 | 6 | 118,006 | 1,994 (1.67 ± 0.12)^{d} | 3,211,504 | 18,131 (0.56 ± 0.04)^{d} |
| 4 × 7.5 | 6 | 6 | 119,563 | 437 (0.37 ± 0.06) | 4,708,513 | 11,959 (0.25 ± 0.02)^{f} |
| 4 × 180 | 6 | 6 | 117,662 | 2,338 (1.95 ± 0.26)^{d} | 1,064,685 | 7,945 (0.75 ± 0.04)^{d,e} |
| 4 × (7.5 + 180) | 6 | 6 | 117,887 | 2,113 (1.76 ± 0.17)^{d} | 1,246,772 | 10,076 (0.81 ± 0.07)^{d,e} |
| 4 × 7.5 | 28 | 4 | 79,725 | 275 (0.35 ± 0.06) | 4,719,142 | 6,896 (0.22 ± 0.01) |
| 4 × 180 | 28 | 6 | 119,080 | 920 (0.77 ± 0.18)^{d} | 3,902,522 | 28,089 (0.72 ± 0.15)^{d} |
| 4 × (7.5 + 180) | 28 | 6 | 119,057 | 943 (0.79 ± 0.13)^{d} | 3,159,295 | 22,067 (0.71 ± 0.09)^{d} |

^{a}Once a week with 6 hr of separation for multiple daily doses.
^{b}In relation to 4th irradiation.
^{c}For each mouse, an overall average was obtained by pooling the values from the tree collection points
^{d}P < 0.0001 vs sham
^{e}P < 0.0001 vs 3 weeks
^{f}P < 0.02 vs sham

TABLE 7a

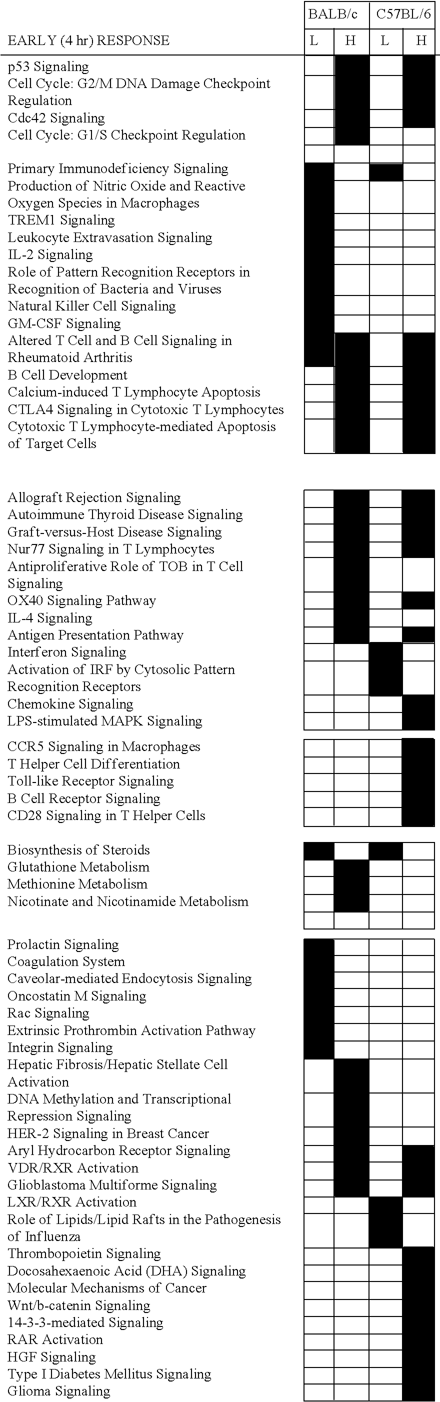

TABLE 7b

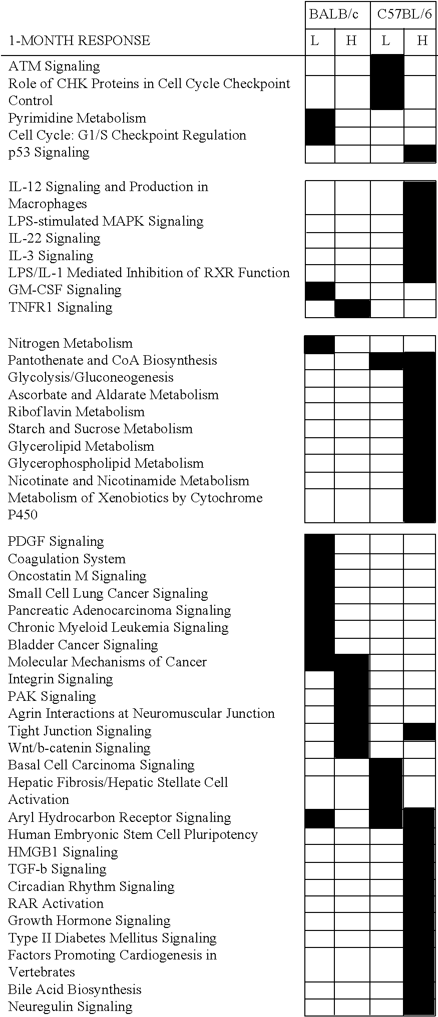

TABLE 8
Baseline expression differences in blood of BALB/c vs C57BL/6 mice
Fold changes listed in log₂ relative average expression in four C57BL/6 mice

| | | | BALB/c | BALB/c | BALB/c | BALB/c |
|---|---|---|---|---|---|---|
| Gene | Function | Taqman AOD ID | LD #1 | LD #2 | LD #3 | LD #4 |
| PARP3 | DNA stability | Mm00467486_m1 | −1.27 | −1.17 | −0.83 | −1.12 |
| PARP3 | DNA stability | Mm01232604_m1 | −1.80 | −1.00 | −1.14 | −1.57 |
| MSH5 | DNA stability | Mm00488974_m1 | 2.34 | 2.65 | 2.21 | 2.34 |
| SMC6 | DNA stability | Mm01273370_m1 | 1.59 | 1.80 | 1.74 | 2.32 |

TABLE 11
Four categories of genes and their Unigene and
GeneIDs from GenBank

| | | | |
|---|---|---|---|
| Affy ID | Gene | Unigene | Gene ID |

ROW 1

| | | | |
|---|---|---|---|
| 222077_s_at | RACGAP1 | Hs.505469 | 29127 |
| 203225_s_at | RFK | Hs.37558 | 55312 |
| 221619_s_at | MTCH1 | Hs.485262 | 23787 |
| 218206_x_at | SCAND1 | Hs.584909 | 51282 |
| 218009_s_at | PRC1 | Hs.366401 | 9055 |
| 219983_at | HRASLS | Hs.36761 | 57110 |
| 204026_s_at | ZWINT | Hs.591363 | 11130 |
| 200842_s_at | EPRS | Hs.497788 | 2058 |

ROW 2

| | | | |
|---|---|---|---|
| 209380_s_at | ABCC5 | Hs.743953 | 10057 |
| 201597_at | COX7A2 | Hs.70312 | 1347 |
| 213424_at | KIAA0895 | Hs.6224 | 23366 |
| 202733_at | P4HA2 | Hs.519568 | 8974 |
| 212070_at | GPR56 | Hs.513633 | 9289 |
| 213520_at | RECQL4 | Hs.31442 | 9401 |
| 205656_at | PCDH17 | Hs.106511 | 27253 |
| 201385_at | DHX15 | Hs.696074 | 1665 |
| 200942_s_at | HSBP1 | Hs.250899 | 3281 |
| 202425_x_at | PPP3CA | Hs.435512 | 5530 |
| 202534_x_at | DHFR | Hs.592364 | 1719 |

ROW 3

| | | | |
|---|---|---|---|
| 204156_at | SIK3 | Hs.167451 | 23387 |
| 211663_x_at | PTGDS | Hs.446429 | 5730 |
| 214946_x_at | FAM21B | Hs.449662 | 55747 |
| 203143_s_at | KIAA0040 | Hs.518138 | 9674 |
| 212614_at | ARID5B | Hs.535297 | 84159 |
| 208937_s_at | ID1 | Hs.504609 | 3397 |
| 219132_at | PELI2 | Hs.657926 | 57161 |
| 205898_at | CX3CR1 | Hs.78913 | 1524 |
| 220038_at | SGK3 | Hs.613417 | 23678 |

ROW 4

| | | | |
|---|---|---|---|
| 205987_at | CD1C | Hs.132448 | 911 |
| 208611_s_at | SPTAN1 | Hs.372331 | 6709 |
| 200848_at | AHCYL1 | Hs.743973 | 10768 |
| 206540_at | GLB1L | Hs.181173 | 79411 |
| 203799_at | CD302 | Hs.130014 | 9936 |
| 205977_s_at | EPHA1 | Hs.89839 | 2041 |
| 217235_x_at | IGLL1/IGLL5 | Hs.348935 | 3543 |
| | | Hs.625768 | 100423062 |

What is claimed is:

1. A panel of genetic probes for determining higher predicted probability of disease free survival in a patient, said panel comprising genetic probes to detect the genes or gene products of BMP2K, CBX7, CLASP2, PRDX2, PRDX3, RAB6B, RPS6, RUNX1 and SLC15A2.

2. A method for identifying a cancer patient with higher predicted probability of disease free survival, comprising: (a) measuring the amplification or expression level of each gene in said panel of claim 1 in a sample from a patient; and (b) determining a total amplification or expression level of said panel by adding together the measurements from Step (a); and (c) comparing said total in Step (b) to a median of total amplification or expression level of said panel of genes in a normal tissue sample or a reference amplification or expression level, whereby a below-median expression level indicates a patient that has a higher predicted probability of disease free survival.

3. The method of claim 1 further comprising: (d) prescribing methods to reduce exposure to low dose radiation to patients with an above-median expression level.

4. The method of claim 3 where the method to reduce exposure to low dose radiation is obtaining extra shielding for said patient.

5. The method of claim 4 where the method to reduce exposure to low dose radiation is seeking an alternative to airport security scanners.

6. A panel of genetic probes for determining susceptibility to low dose ionizing radiation (LD)-induced cancer in a patient, said panel comprising genetic probes to detect the genes or gene products of BMP2K, CBX7, CLASP2, PRDX2, PRDX3, RAB6B, RPS6, RUNX1 and SLC15A2.

7. A method for identifying a patient with susceptibility to low dose ionizing radiation (LD)-induced cancer, comprising: (a) measuring the amplification or expression level of each gene in said panel of claim 6 in a sample from a patient before exposure to LD; and (b) determining a total amplification or expression level of said panel by adding together the measurements from Step (a); (c) comparing said total in Step (b) to a median of total amplification or expression level of said panel of genes in a normal tissue sample or a reference amplification or expression level, whereby a below-median expression level indicates a patient is resistant to LD-induced cancers.

8. A panel of genetic probes for determining higher predicted probability of disease free survival in a patient, said panel comprising genetic probes to detect the genes or gene products of MAGOHB, PAPOLA, PNPT1, POP4, PPIH, RBM39, SUPT16H, TXNL4A, EIF2S1, GNA13, GNB1, HLA-DRA, RAD23A, SMC6, ABCB10, ABCF1, BAT5, C17orf95, C19orf56, C5orf22, CAP1, CHCHD3, CHCHD4, CLDND1, DDX19A, DNAJC10, GADD45GIP1, GBP1, HLA-B, KIF5B, MCART1, MCM6, MTFR1, NRD1, PDK1, PDXDC1, PEBP1, PHF20, PI4K2B, PIGO, PPME1, SAPS3, SCAND1, SHC1, SNX6 and TM2D2.

9. A method for identifying a cancer patient with higher predicted probability of disease free survival, comprising: (a) measuring the amplification or expression level of each gene in said panel of claim 8 in a sample from a patient; and (b) determining a total amplification or expression level of said panel by adding together the measurements from Step (a); and (c) comparing said total in Step (b) to a median of total amplification or expression level of said panel of genes in a normal tissue sample or a reference amplification or expression level, whereby an above-median expression indicates a patient that has a higher predicted probability of disease free survival.

10. A panel of genetic probes for determining susceptibility to low dose ionizing radiation (LD)-induced cancer in a patient, said panel comprising genetic probes to detect the genes or gene products of MAGOHB, PAPOLA, PNPT1, POP4, PPIH, RBM39, SUPT16H, TXNL4A, EIF2S1, GNA13, GNB1, HLA-DRA, RAD23A, SMC6, ABCB10, ABCF1, BAT5, C17orf95, C19orf56, C5orf22, CAP1, CHCHD3, CHCHD4, CLDND1, DDX19A, DNAJC10, GADD45GIP1, GBP1, HLA-B, KIF5B, MCART1, MCM6, MTFR1, NRD1, PDK1, PDXDC1, PEBP1, PHF20, PI4K2B, PIGO, PPME1, SAPS3, SCAND1, SHC1, SNX6 and TM2D2.

11. A method for identifying a patient with susceptibility to low dose ionizing radiation (LD)-induced cancer, comprising: (a) measuring the amplification or expression level of each gene in said panel of claim 10 in a sample from a patient before exposure to LD; and (b) determining a total amplification or expression level of said panel by adding together the measurements from Step (a); and (c) comparing said total in Step (b) to a median of total amplification or expression level of said panel of genes in a normal tissue sample or a reference amplification or expression level, whereby an above-median expression indicates a patient is resistant to LD-induced cancers.
